(19) United States
(12) Patent Application Publication
(10) Pub. No.: US 2023/0212120 A1
(43) Pub. Date: Jul. 6, 2023

(54) SMALL MOLECULE INHIBITORS OF GRK5 AND GRK5 SUBFAMILY MEMBERS AND USES THEREOF

(71) Applicants: The Regents of the University of Michigan, Ann Arbor, MI (US); Purdue Research Foundation, West Lafayette, IN (US)

(72) Inventors: John J.G. Tesmer, West Lafayette, IN (US); Rachel Rowlands, Ann Arbor, MI (US); Andrew White, Ann Arbor, MI (US)

(21) Appl. No.: 17/928,075

(22) PCT Filed: May 27, 2021
(86) PCT No.: PCT/US2021/034553

§ 371 Date: Nov. 28, 2022

Related U.S. Application Data
(60) Provisional application No. 63/032,179, filed on May 29, 2020.

(51) Int. Cl. C07D 209/34; C07D 403/06; C07D 405/12; C07D 405/14

(52) U.S. Cl. CPC C07D 209/34; C07D 403/06; C07D 405/12; C07D 405/14

(57) ABSTRACT

This invention is in the field of medicinal chemistry. In particular, the invention relates to a new class of small-molecules having an indolinone structure which function as inhibitors of GRK5, and their use as therapeutics for the treatment of heart conditions and cancer.

### CROSS-REFERENCE TO RELATED APPLICATION

This application claims priority to U.S. Provisional Patent Application No. 63/032,179 filed May 29, 2020, the contents of which are incorporated by reference in its entirety.

### FIELD OF THE INVENTION

This invention is in the field of medicinal chemistry. In particular, the invention relates to anew class of small-molecules having an indolinone structure which function as inhibitors of GRK5, and their use as therapeutics for the treatment of heart conditions and cancer.

### INTRODUCTION

G protein-coupled receptors (GPCRs) modulate cellular events in response to extracellular signals. GPCR related kinases (GRKs) selectively recognize and phosphorylate activated GPCRs, leading to their desensitization and internalization, a process critical for maintaining cellular homeostasis. The seven known GRKs (GRK1-7), are classified through structural and sequence similarity, into three subfamilies; the GRK1 (GRK1 and 7), GRK2 (GRK2 and 3), and GRK4 (GRK4, 5, and 6) subfamilies (see, Pitcher, J. A.; et al., Annu. Rev. Biochem. 1998, 67 (1), 653-692). GRK1 and 7 are expressed primarily in the retina and GRK4 in the testes, whereas GRK2, 3, 5, and 6 are more ubiquitously expressed (see, Ferguson, S. S. G. Pharmacol. Rev. 2001, 53 (1), 1-24). Of these kinases, GRK2 and GRK5 are the two isoforms found in highest concentration in cardiovascular tissue. The GRK5 subfamily is part of a larger superfamily that includes the catalytic domains of serine/threonine kinases, protein tyrosine kinases, RIO kinases, aminoglycoside phosphotransferase, choline kinase, and phosphatidylinositide 3-kinases.

GRK2 and GRK5 are considered therapeutic targets for various disease states such as: cancer, inflammation, Parkinson's disease, Alzheimer's disease, heart failure and hypertrophic cardiomyopathy (see, Jiang, L.-P.; et al., Cell Death Dis. 2018, 9 (3), 295; Belmonte, S. L.; Blaxall, B. C. Circ. Res. 2012, 111 (8), 957-958; Brinks, H.; Koch, W. J. Today Dis. Mech. 2010, 7 (2), e129-e134; Lymperopoulos, A.; et al., Nat. Med. 2007, 13 (3), 315-323; Nogues, L.; et al., Semin. Cancer Biol. 2018, 48, 78-90). GRK5 is further unique among the GRKs because it undergoes a Ca²⁺-calmodulin-dependent nuclear localization event. Once translocated to the nucleus, GRK5 can phosphorylate histone deacetylase 5 (HDAC5) which is responsible for increasing transcription of genes associated with hypertrophic cardiomyopathy. In studies where GRK5 was knocked down, cardiomyocytes were protected from hypertrophic cardiomyopathy (see, Huang, Z. M.; et al., Front. Biosci. Landmark Ed. 2011, 16, 3047-3060). The influence of GRK5 in progressive heart failure and hypertrophic cardiomyopathy remains unclear, in part because GRK2 can also mediate hypertrophic responses (see, Schlegel, P.; et al., PLoS One 2017, 12 (7), e0182110; Lieu, M.; et al., Expert Opin Ther Targets 2019, 23 (3), 201-214) there are no available GRK5-selective and GRK5 subfamily selective compounds to test mechanistic hypotheses.

Accordingly, GRK5 and GRK5 subfamily (e.g., GRK4, GRK5, and GRK6) selective compounds and related improved treatment methods for treating conditions associated with GRK5 activity (e.g., heart conditions and cancer) are needed.

The present invention addresses this need.

### SUMMARY OF THE INVENTION

The ability of G protein-coupled receptor (GPCR) kinases (GRKs) to regulate desensitization of GPCRs has made GRK2 and GRK5 attractive targets for treating diseases such as heart failure and cancer. Previous work showed Cys474 can be used as a covalent handle to achieve selective inhibition of GRK5 over GRK2 subfamily members. Despite these initial efforts, potency of the most selective inhibitors remained modest. Experiments conducted during the course of developing embodiments for the present invention involved virtual screening and validation efforts that uncovered a new indolinone scaffold to be used as a vehicle for selective covalent GRK5 inhibition, as well as a rational design campaign that yielded compounds that are essentially specific for GRK5 subfamily members and of low nanomolar potency. Indeed, such experiments resulted in the designing, synthesizing, and biological evaluation of compounds having an indolinone structure as inhibitors of GRK5 and GRK5 subfamily members and their potential use as therapeutics against heart disease and cancer.

Indeed, the GRK5 subfamily member inhibitors described herein can be considered as potential therapeutics for the treatment of heart related conditions, disorders and/or diseases (e.g., any condition related to aberrant heart function) (e.g., myocardial ischemia, ischemia-induced heart failure, chronic heart failure (CHF), ischemia without heart failure, hypertrophic cardiomyopathy, dilated cardiomyopathy (DCM), cardiac arrest, congestive heart failure, stable angina, unstable angina, myocardial infarction, coronary artery disease, valvular heart disease, ischemic heart disease, acute coronary syndromes, atherosclerotic coronary artery disease, reduced ejection fraction, reduced myocardial perfusion, maladaptive cardiac remodeling, maladaptive left ventricle remodeling, reduced left ventricle function, left heart failure, right heart failure, backward heart failure, forward heart failure, systolic dysfunction, diastolic dysfunction, increased or decreased systemic vascular resistance, low-output heart failure, high-output heart failure, dyspnea on exertion, dyspnea at rest, orthopnea, tachypnea, paroxysmal nocturnal dyspnea, dizziness, confusion, cool extremities at rest, exercise intolerance, easy fatigue ability, peripheral edema, nocturia, ascites, hepatomegaly, pulmonary edema, cyanosis, laterally displaced apex beat, gallop rhythm, heart murmurs, parasternal heave, and pleural effusion).

The GRK5 and GRK5 subfamily (e.g., GRK4, GRK5, and GRK6) inhibitors of the present invention have been also implicated as therapeutics for the treatment of hyperproliferative diseases such as cancer (e.g., breast cancer).

Accordingly, this invention relates to a new class of small-molecules having an indolinone structure which function as inhibitors of GRK5 protein and GRK5 subfamily (e.g., GRK4, GRK5, and GRK6) members, and their use as therapeutics for the treatment of disorders related to GRK5 and GRK5 subfamily member activity (e.g., heart conditions) (e.g., cancer).

In a particular embodiment, compounds encompassed within the following formulas are provided

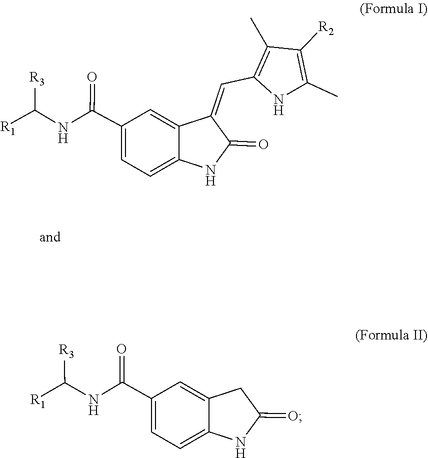

including pharmaceutically acceptable salts, solvates, and/or prodrugs thereof.

Formula I is not limited to a particular chemical moiety for R1, R2 and R3. In some embodiments, the particular chemical moiety for R1, R2 and R3 independently include any chemical moiety that permits the resulting compound to inhibit GRK5 activity. In some embodiments, the particular chemical moiety for R1, R2 and R3 independently include any chemical moiety that permits the resulting compound to inhibit GRK5 subfamily (e.g., GRK4, GRK5, and GRK6) activity. In some embodiments, the particular chemical moiety for R1, R2 and R3 independently include any chemical moiety that permits the resulting compound to inhibit histone deacetylase 5 (HDAC5) phosphorylation. In some embodiments, the particular chemical moiety for R1, R2 and R3 independently include any chemical moiety that permits the resulting compound to inhibit GRK5 subfamily activity while not significantly affecting GRK2 activity. In some embodiments, the particular chemical moiety for R1, R2 and R3 independently include any chemical moiety that permits the resulting compound to bind a GRK5 protein at the Cys474 position or the equivalent position in other GRK5 subfamily members. In some embodiments, the particular chemical moiety for R1, R2 and R3 independently include any chemical moiety that permits the resulting compound to inhibit GRK5 related interaction with IκBα which thereby inhibits NFKB-mediated transcriptional responses. In some embodiments, the particular chemical moiety for R1, R2 and R3 independently include any chemical moiety that permits the resulting compound to inhibit GRK5 related phosphorylation of p53 and regulates p53-mediated apoptosis in response to DNA damage.

In some embodiments, R1 is selected from hydrogen,

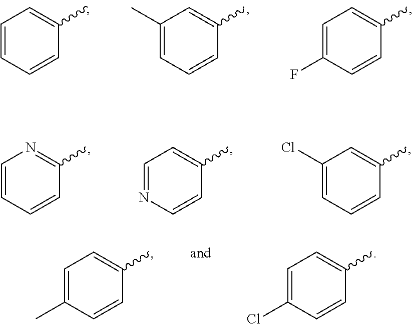

In some embodiments, R2 is selected from hydrogen,

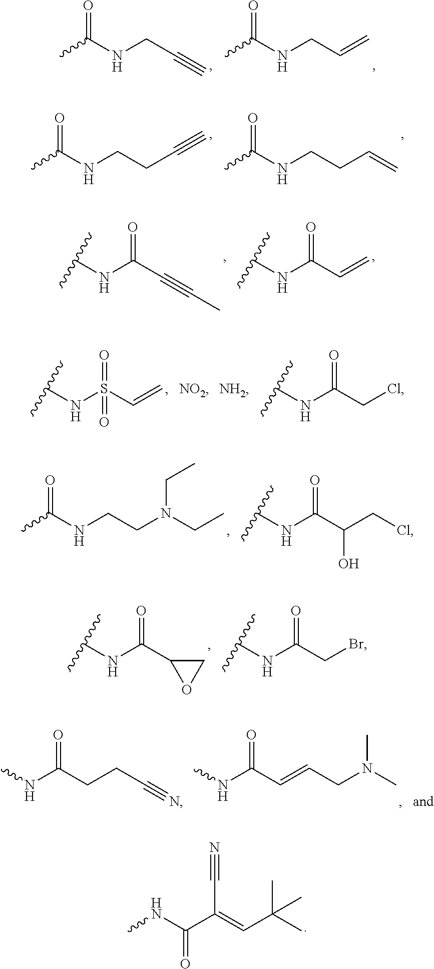

In some embodiments, R3 is selected from hydrogen,

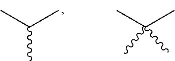

(e.g., such that the resulting compound is either

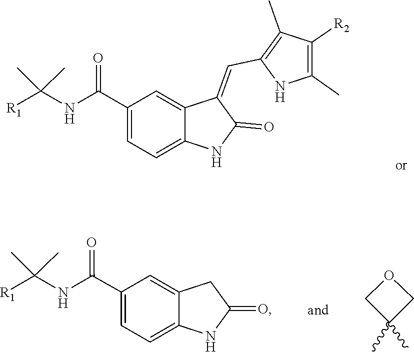

(e.g., such that the resulting compound is either

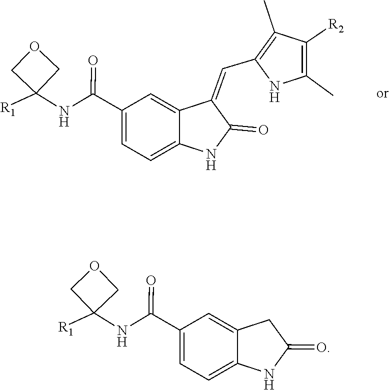

Certain indolinone compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In some the embodiments, the compound encompassed within Formula I is recited in Tables I, II and Example 1.

The invention further provides processes for preparing any of the compounds of the present invention.

The compounds of the invention are useful for the treatment, amelioration, or prevention of disorders associated with GRK5 activity, such as those responsive to GRK5 activity inhibition and/or GRK5 subfamily activity inhibition. In certain embodiments, the compounds can be used to treat, ameliorate, or prevent cancer that is associated with GRK5 activity (e.g., breast cancer). In certain embodiments, the compounds can be used to treat, ameliorate, or prevent heart conditions related to GRK5 activity (e.g., one or more of cardiac failure, cardiac hypertrophy, and hypertension).

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents, e.g., agents useful in treating conditions associated with GRK5 activity and/or GRK5 subfamily activity (e.g., heart conditions, cancer).

In certain embodiments, the present invention provides methods of inhibiting GRK5 activity in a cell, comprising contacting the cell with a compound as disclosed herein (e.g., Formula I) or a pharmaceutically acceptable salt thereof, as defined herein, or a pharmaceutical formulation thereof, in an amount effective to inhibit GRK5. In some embodiments, the cell is a myocyte, such as a cardiomyocyte. In some embodiments, the cell is a cancer cell, such as a breast cancer cell or a cancer cell associated with GRK5 activity. The contacting can occur, for example, in vivo. In some embodiments, the contacting comprises administering to a subject in need thereof. In some embodiments, the subject suffers from heart disease. The heart disease can be one or more of cardiac failure, cardiac hypertrophy, and hypertension. In some embodiments, the subject suffers from cancer (e.g., breast cancer).

The present disclosure further provides bifunctional compounds that function to recruit endogenous proteins to an E3 Ubiquitin Ligase for degradation, and methods of using the same. In particular, the present disclosure provides bifunctional or proteolysis targeting chimeric (PROTAC) compounds, which find utility as modulators of targeted ubiquitination of a variety of polypeptides and other proteins, which are then degraded and/or otherwise inhibited. An exemplary advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as any type of cancer or heart condition characterized with GRK5 activity.

In an additional aspect, the disclosure provides bifunctional or PROTAC compounds, which comprise an E3 Ubiquitin Ligase binding moiety (e.g., a ligand for an E3 Ubquitin Ligase or “ULM” group), and a moiety that binds a target protein (e.g., a protein/polypeptide targeting ligand or “PTM” group) (e.g., a GRK5 inhibitor) such that the target protein/polypeptide is placed in proximity to the ubiquitin ligase to effect degradation (and inhibition) of that protein. In certain embodiments, the PTM is any of the compounds as described herein showing inhibitory activity against GRK5 activity. In some embodiments, the ULM is a VHL, cereblon, mouse double minute 2 (MDM2), and/or inhibitor of apoptosis protein (IAP) E3 ligase binding moiety. For example, the structure of the bifunctional compound can be depicted as PTM-ULM.

The respective positions of the PTM and ULM moieties, as well as their number as illustrated herein, is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as PTM-L-ULM, where PTM is a protein/polypeptide targeting moiety (e.g., any of the compounds as described herein showing inhibitory activity against GRK5), L is a linker, and ULM is a VHL, cereblon, MDM2, or IAP E3 ligase binding moiety binding moiety.

Such embodiments are not limited to a specific type of linker. In some embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical. In some embodiments, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

The ULM group and PTM group may be covalently linked to the linker group through any group which is appropriate and stable to the chemistry of the linker. In exemplary aspects of the present invention, the linker is independently covalently bonded to the ULM group and the PTM group in certain embodiments through an amide, ester, thioester, keto group, carbamate (urethane), carbon or ether, each of which groups may be inserted anywhere on the ULM group and PTM group to provide maximum binding of the ULM group on the ubiquitin ligase and the PTM group on the target protein to be degraded. In certain aspects where the PTM group is a ULM group, the target protein for degradation may be the ubiquitin ligase itself. In certain exemplary aspects, the linker may be linked to an optionally substituted alkyl, alkylene, alkene or alkyne group, an aryl group or a heterocyclic group on the ULM and/or PTM groups.

In certain embodiments, the compounds as described herein comprise multiple ULMs, multiple PTMs, multiple chemical linkers, or any combinations thereof.

In some embodiments, the present invention provides a method of ubiquitinating/degrading GRK5 activity in a cell comprising administering a bifunctional compound as described herein comprising an ULM and a PTM, in certain embodiments linked through a linker moiety, as otherwise described herein, wherein the ULM is coupled to the PTM and wherein the ULM recognizes a ubiquitin pathway protein and the PTM recognizes the target protein such that degradation of the target protein occurs when the target protein is placed in proximity to the ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cells of a patient.

### BRIEF DESCRIPTION OF THE DRAWINGS

FIG. 1: Lead compounds 1 and 5a were derived from a virtual screen depicted by the screening funnel.

FIG. 2: Lead compound, 5a (yellow carbons), docked into the GRK5 (blue)/GRK6 (purple) overlay model. The diamine moiety is solvent exposed, facing the AST loop where Cys474 (green carbons) is located.

FIG. 3: Intact protein MS traces for 5a-9f. MS indicates that only 9e covalently engages GRK5 within 30 minutes (raspberry), whereas the other compounds do not covalently label GRK5 within this time frame.

FIG. 4: Incubation of the original warhead series (5c-5f, 9a-9c, 9o-9t) for 3 h, rather than the original 30 min time point. Increased time also allows 9c to form a covalent engagement, illustrating the role of kinetics (Kₒₙ) in the formation of covalent interactions.

FIG. 5: Intact protein mass spectrometry for key compounds 9e, 9g, and 9j. (a) GRK5 and GRK5+9e incubated for 30 mins. 9e demonstrates full labelling of GRK5 in the MS trace (black), (b) GRK5 and GRK5+9g incubated for 30 mins. 9g only labels 50% of GRK5 present in this sample, as indicated by MS trace (pink), (c) GRK5 and GRK5+9j incubated for 30 mins. 9j fully labels GRK5 in this timeframe as indicated by MS trace (red), (d) GRK5-C474S mutant and GRK5-C474S+9e incubated for 30 mins. 9e does not label GRK5-C474S confirming these compounds are engaging Cys474 as the covalent handle.

FIG. 6: Tandem MS analysis of GRK5 incubated with 9c (incubation at 3 h). Specific modifications of Cys474 by 9c are highlighted in green within the table. Other modifications at solvent exposed Cys145 and Cys520 (represented as spheres in figure below with yellow sulfurs) are considered the result of concentration dependent labelling and do not represent biologically important labelling events.

FIG. 7: All intact MS data for 5b, 9g-9n, and 10a-10b. Only a handful of these compounds, namely, 9j and 9g (4-fluoro, bromoketone respectively), were able to form a covalent interaction, as shown by an appropriate shift in molecular weight of the GRK5 peak.

FIG. 8: Metabolic stability of 9c, 9e, and 9j within mouse liver microsomes (MLMs) and human liver microsomes (HLMs). Stop light coding indicates that all three compounds had poor stability, less than 10 minutes, within mouse models. However, stability in HLMs increases 1.4-fold when a 4-fluoro pendant us added to protect the phenyl ring from metabolism.

FIG. 9: Intact MS of benzylic analogues (9q-9t) incubated overnight. Three of five of these compounds can form a covalent interaction. The other two compounds are assumed to have a slower Kₒₙ rate that necessitates a longer incubation period to form the desired covalent interaction. (a) all intact MS spectra shown, (b) GRK5 (blue) and GRK5+9q show that this compound completes a partial labelling at 8 hours, (C) GRK5 (blue) and GRK5+9r shows that this compound completes a partial labelling at 8 hours, (d) GRK5 (blue) and GRK5+9s show that this compound completes labelling of GRK5 at 8 hours.

FIG. 10: Kinome-wide selectivity panel for 9g at concentrations of (a) 1 μM, (b) 0.1 μM, and (c) 0.01 μM. A clear dose response is evident, with fewer kinases inhibited at the lowest concentration of 9g.

FIG. 11: Kinome panel for 5c, tested at 1 μM. Major targets are GRK5, GRK6, PDGFR, and VEGFR, among other tyrosine kinases.

### DETAILED DESCRIPTION OF THE INVENTION

GRK5 is an important member of the threonine/serine kinase family that phosphorylates GPCRs as part of feedback inhibition of GPCRs in signal transduction. The in vivo physiological functions of GRK5 have been ascribed to its kinase activity, phosphorylating and desensitizing specific GPCRs, as well as its kinase independent function and targeting to non-GPCR substrates. GRK5 interacts with IκBα and inhibits NFKB-mediated transcriptional responses. GRK5 also phosphorylates p53 and regulates p53-mediated apoptosis in response to DNA damage. G protein-coupled receptor kinases (GRKs) specifically phosphorylate serine/threonine residues located on the cytoplasmic loops and C terminus of the receptors. Phosphorylation of a GPCR by GRK promotes the high affinity binding of arrestins and induces receptor internalization, and thus feed-back inhibits GPCR responses to extracellular signals. The in vivo physiological functions of various subtypes of GRKs (GRK1-7) have been previously ascribed to phosphorylating and desensitizing specific GPCRs.

This invention relates to a new class of small-molecules having an indolinone structure which function as inhibitors of GRK5 protein, and their use as therapeutics for the treatment of disorders related to GRK5 activity (e.g., heart conditions) (e.g., cancer).

In a particular embodiment, compounds encompassed within the following formulas are provided:

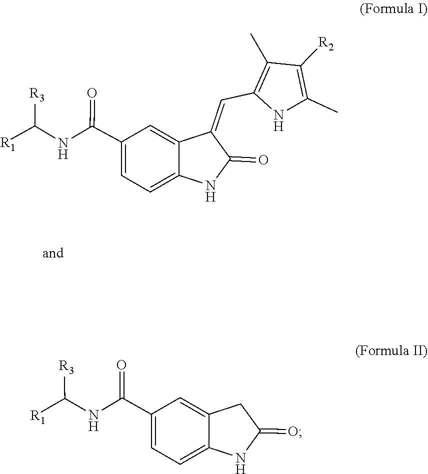

including pharmaceutically acceptable salts, solvates, and/or prodrugs thereof.

Formula I is not limited to a particular chemical moiety for R1, R2 and R3. In some embodiments, the particular chemical moiety for R1, R2 and R3 independently include any chemical moiety that permits the resulting compound to inhibit GRK5 activity and/or GRK5 subfamily (e.g., GRK4, GRK5, and GRK6) activity. In some embodiments, the particular chemical moiety for R1, R2 and R3 independently include any chemical moiety that permits the resulting compound to inhibit GRK5 activity while not affecting GRK2 activity. In some embodiments, the particular chemical moiety for R1, R2 and R3 independently include any chemical moiety that permits the resulting compound to bind a GRK5 protein at the Cys474 position. In some embodiments, the particular chemical moiety for R1, R2 and R3 independently include any chemical moiety that permits the resulting compound to inhibit GRK5 related interaction with IκBα which thereby inhibits NFKB-mediated transcriptional responses. In some embodiments, the particular chemical moiety for R1, R2 and R3 independently include any chemical moiety that permits the resulting compound to inhibit GRK5 related phosphorylation of p53 and regulates p53-mediated apoptosis in response to DNA damage.

In some embodiments, R1 is selected from hydrogen,

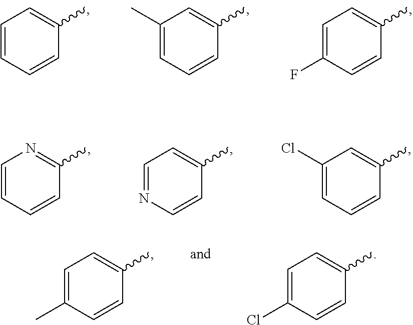

In some embodiments, R2 is selected from hydrogen,

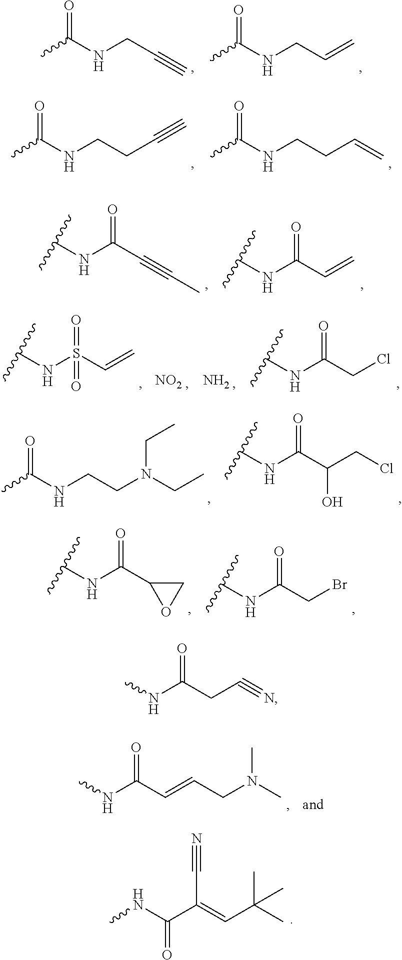

In some embodiments, R3 is selected from hydrogen,

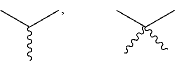

(e.g., such that the resulting compound is either

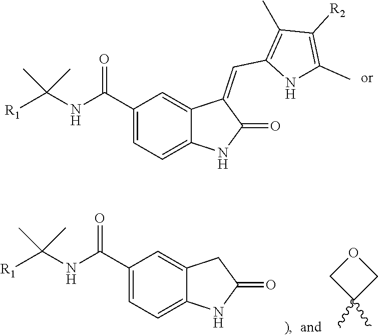

(e.g., such that the resulting compound is either

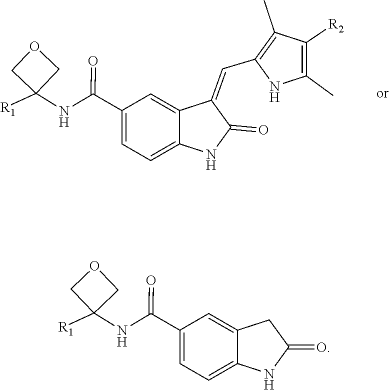

Certain indolinone compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In some the embodiments, the compound encompassed within Formula I is recited in Tables I, II and Example 1.

The invention further provides processes for preparing any of the compounds of the present invention.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, heart conditions, cancer, and any condition associated with GRK5 activity.

Examples of such heart related conditions, disorders and/or diseases include, but are not limited to, any condition related to aberrant heart function, myocardial ischemia, ischemia-induced heart failure, chronic heart failure (CHF), ischemia without heart failure, hypertrophic cardiomyopathy, dilated cardiomyopathy (DCM), cardiac arrest, congestive heart failure, stable angina, unstable angina, myocardial infarction, coronary artery disease, valvular heart disease, ischemic heart disease, acute coronary syndromes, atherosclerotic coronary artery disease, reduced ejection fraction, reduced myocardial perfusion, maladaptive cardiac remodeling, maladaptive left ventricle remodeling, reduced left ventricle function, left heart failure, right heart failure, backward heart failure, forward heart failure, systolic dysfunction, diastolic dysfunction, increased or decreased systemic vascular resistance, low-output heart failure, high-output heart failure, dyspnea on exertion, dyspnea at rest, orthopnea, tachypnea, paroxysmal nocturnal dyspnea, dizziness, confusion, cool extremities at rest, exercise intolerance, easy fatigue ability, peripheral edema, nocturia, ascites, hepatomegaly, pulmonary edema, cyanosis, laterally displaced apex beat, gallop rhythm, heart murmurs, parasternal heave, and pleural effusion.

Examples of cancer include any type of cancer associated with GRK5 activity. A non-limiting exemplary list of different types of cancer, but is not limited to, pancreatic cancer, ovarian cancer, breast cancer, prostate cancer, lymphoma, skin cancer, colon cancer, melanoma, malignant melanoma, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer cells being treated are metastatic. In other embodiments, the cancer cells being treated are resistant to anticancer agents.

Some embodiments of the present invention provide methods for administering an effective amount of a compound of the invention and at least one additional therapeutic agent (including, but not limited to, any agent useful in treating heart conditions and/or cancer).

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### Examples

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

A set of GRK5-selective pyrrolopyrimidine based inhibitors using a covalent strategy was previously established (see, Rowlands, R.; Cato, M. C.; Waldschmidt, H. V.; Bouley, R. A.; Chen, Q.; Avramova, L.; Larsen, S. D.; Tesmer, J. J. G.; White. Structure-Based Design of Selective, Covalent G Protein-Coupled Receptor Kinase 5 Inhibitors|ACS Medicinal Chemistry Letters). This effort established that Cys474, located on the active site tether (AST, a loop that packs over the ATP binding site in AGC kinases), can serve as a covalent handle that could be exploited to achieve GRK5 selectivity. However, the affinities of the best compounds were modest at best (low μM). Because the intrinsic affinity of the compound plays an important role in dictating the concentration of the protein-inhibitor covalent complex, which must persist long enough for a covalent reaction to occur, the discovery of a more intrinsically potent scaffold was prioritized.

To this end, experiments were conducted involving a virtual, knowledge-based screen (see, Ekins, S.; et al., Br. J. Pharmacol. 2007, 152 (1), 9-20) using the overlay model of GRK5/GRK6 previously described (PDB entries 4WNK and 3NYN respectively) (see, Boguth, C. A.; et al., EMBO J. 2010, 29 (19), 3249-3259; Homan, K. T.; et al., J. Biol. Chem. 2015, 290 (34), 20649-20659). The enriched screening library was compiled from Chembl and Maybridge chemical libraries and refined using Pipeline Pilot to exclude compounds that are potentially reactive through unmasking of reactive intermediates (resultant library of 2408 compounds). Making use of the crystal structure of GRK5, a pharmacophore was developed based on the binding mode of CCG-215022 (see, Homan, K. T., et la., J Biol Chem 290, 20649-20659). The enriched library was then screened against GRK5, using the pharmacophore to refine the search parameters. All initial hits were then clustered into series and individually docked without the pharmacophore refinement to validate the results seen in the initial virtual screen. Only those compounds which were able to replicate their binding modes were selected as initial lead compounds.

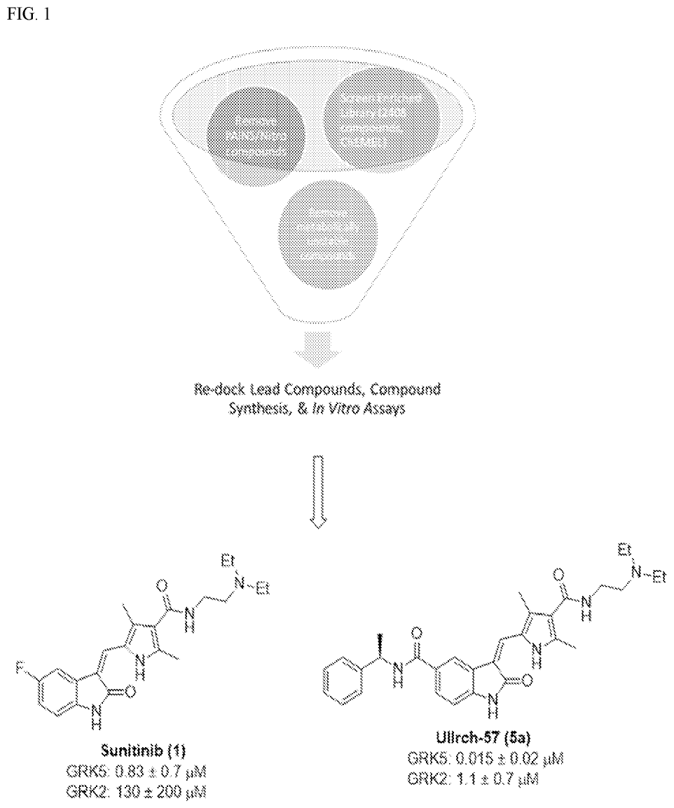

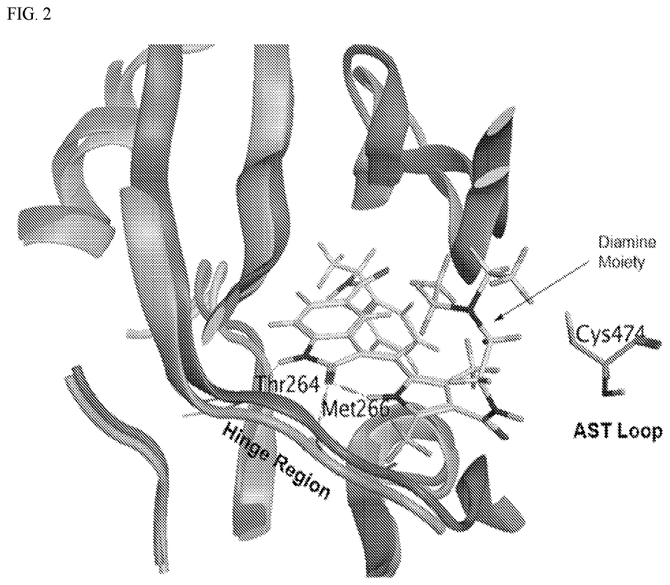

From these search parameters two new scaffolds were discovered, an aminopyridine scaffold and an indolinone-based scaffold exemplified by Sunitinib (1) and Ullrich-57 (FIG. 1) (see, Cho, S. Y.; Bioorg. Med. Chem. Lett. 2013, 23 (24), 6711-6716; Ullrich, A.; WO2015022437 A1, Feb. 19, 2015). Given the difficulty of synthesis for the aminopyridine series, experiments were conducted that chose to explore the indolinone series. There was a focus on Ullrich-57 (5a), which was reported to have nanomolar activity against GRK5 (GRK5 IC₅₀=<0.1 μM) but no data was reported for GRK2 (see, WO2015022437). From the model, it is clear that 5a binds the hinge region in an advantageous fashion, with the diamine moiety oriented towards the AST loop (FIG. 2). It was chosen to independently synthesize 5a, and also tested its parent compound, Sunitinib, an FDA-approved chemotherapeutic that targets multiple receptor tyrosine kinases (RTKs) including the platelet-derived growth factor receptor (PDGFR) and vascular endothelial growth factor receptor (VEGFR).

Synthesis for this series began with an amide coupling to give common intermediate 3 (Scheme 1). In a convergent line of synthesis, a secondary amide coupling with the starting material 6, gave intermediate 4a-e. Combining the two lines of synthesis, a Knoevenagel condensation yielded compounds 5a-f. Interestingly, 5a was found to have a 15 nM IC₅₀ for GRK5 and a 74-fold higher IC₅₀ for GRK2, indicating that the base scaffold already has intrinsic selectivity for GRK5 subfamily members. It was hypothesized that the addition of a covalent warhead would further increase the selectivity of this series. Because 5a features a stereocenter, experiments tested whether its enantiomer was equally active. However, it was found that the (S)-enantiomer 5b was over 1000-fold less potent, suggesting that the (R)-enantiomer, 5a, places the methyl pendant in an ideal position, either filling a small lipophilic pocket, or locking the conformation of the molecule in an advantageous pose, or both. All other compounds in this series thus maintain the same stereo-configuration as 5a.

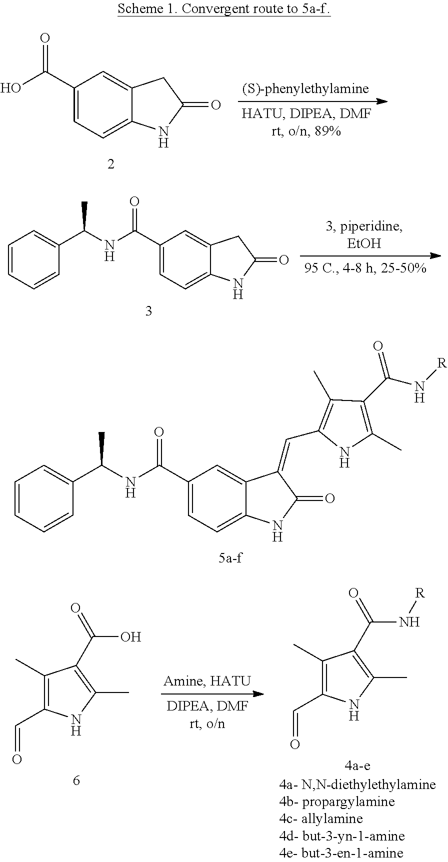

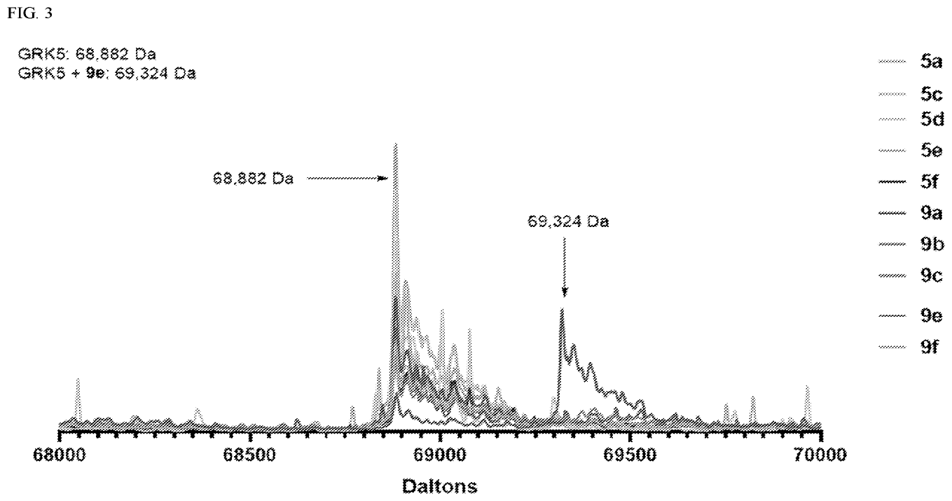

Initial diversification began with the development of alkenyl or alkynyl amines as potential covalent modifiers, that were appended to intermediates 4b-e. Following a Knoevenagel condensation with common intermediate 3, compounds 5c-f were achieved. These changes were meant to introduce softer, weaker electrophiles to the molecule, which could avoid potential off-target side effects of a highly electrophilic covalent warhead. The propargyl analogue, 5c, demonstrated exceptional potency (GRK5 IC₅₀=21 nM) and selectivity over GRK2 (2100-fold). It was speculated that this higher level of selectivity is due to a potential clash between the propargyl warhead and the GRK2 AST and large lobe. However, 5c is not covalent as evaluated by mass spectrometry (FIG. 3). All other amide linked compounds with alkenyl or alkynyl warhead (5c-5f), the selectivity between GRK5 and GRK2 remained between 330-1400 fold selective for GRK5, but the IC₅₀ for GRK5 was higher than that of 5c (Table 1).

TABLE 1
IC₅₀ Values for Indolinone Compounds (μM ± SD)

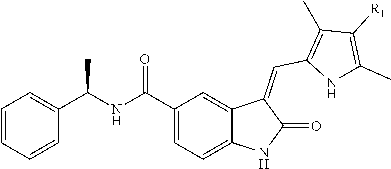

| | | | | | | | GRK2 | | |
|---|---|---|---|---|---|---|---|---|---|
| Compound | | R₁ | GRK5 | | GRK2 | | GRK5 | PKA | |
| Sunitinib | 1 | 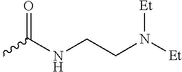 | 0.83 ± 0.7 | (3) | 130 ± 200 | (3) | 150 | ND | |
| CCG 271421 | 5a^{†} | 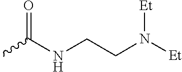 | 0.015 ± 0.02 | (7) | 1.1 ± 0.7 | (4) | 74 | >250 | (2) |
| CCG 273262 | 5b^{†} | 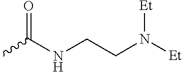 | 1.30 ± 0.1 | (3) | 44 ± 18 | (3) | 32 | ND | |
| CCG 271423 | 5c | 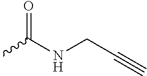 | 0.021 ± 0.01 | (7) | 44 ± 40* | (6) | 2100 | ND | |
| CCG 271424 | 5d | 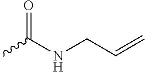 | 0.048 ± 0.008 | (3) | 22 ± 10 | (3) | 460 | >250 | (2) |
| CCG 271441 | 5e | 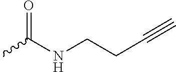 | 0.091 ± 0.04 | (3) | 130 ± 50 | (3) | 1400 | >250 | (2) |
| CCG 271442 | 5f | 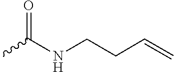 | 1.9 ± 0.05 | (2) | 630 ± 200 | (2) | 330 | ND | |
| CCG 273180 | 9a | 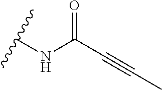 [SMILES: *NC(=O)C#CC] | 2.5 ± 0.8 | (3) | 150 ± 30 | (3) | 61 | ND | |
| CCG 273181 | 9b | 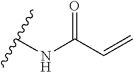 [SMILES: *NC(=O)C=C] | 0.81 ± 0.7 | (4) | 87 ± 30 | (3) | 110 | ND | |
| CCG 273182 | 9c | 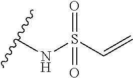 [SMILES: *NS(=O)(=O)C=C] | 0.74 ± 0.6 | (5) | 280 ± 110 | (3) | 370 | ND | |
| CCG | 7a | NO₂ | 0.73 ± 0.5 | (3) | 7.2 ± 3 | (3) | 10. | ND | |
| 273183 | | | | | | | | | |
| CCG 273220 | 9e | 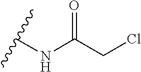 [SMILES: *NC(=O)CCl] | 0.22 ± 0.04 | (3) | 350 ± 100 | (2) | 1500 | >250 | (2) |
| CCG 273221 | 9f | 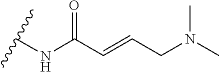 [SMILES: *NC(=O)/C=C/CN(C)C] | 0.36 ± 0.2 | (3) | 17 ± 10 | (2) | 47 | ND | |
| CCG 273464 | 9h | 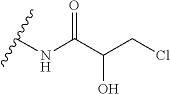 [SMILES: *NC(=O)C(O)CCl] | 0.08 ± 0.03 | | 6.7 ± 5 | (3) | 83 | >250 | (2) |
| CCG 273462 | 10l | 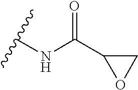 [SMILES: *NC(=O)C1CO1] | 0.74 ± 0.2 | (3) | >250 | (5) | >340 | ND | |
| CCG 273463 | 9g | 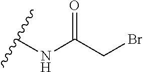 [SMILES: *NC(=O)CBr] | 0.0086 ± 0.003 | (7) | 12 ± 20 | (3) | 1400 | >250 | (2) |
| CCG 273240 | 10a | 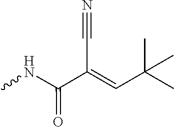 | 0.28 ± 0.1 | (3) | 120 ± 80 | (2) | 430 | >250 | (2) |
| CCG | | — | 0.28 ± 0.1 | (6) | ND | | — | >250 | (2) |
| 215022 | | | | | | | | | |

| | | | | | | | |
|---|---|---|---|---|---|---|---|
| paroxetine | — | ND | 0.78 ± 0.3 | (3) | — | 850 ± 400 | (4) |

All data were fit to a log([inhibitor]) versus response model with variable slope and automatic outlier rejection in GraphPad Prism. Curves that had R squared values less than 0.8 after fitting were omitted. ND, not determined. Values in parentheses indicate the number of independent experimental curves. CCG215022 and paroxetine were included as positive controls for GRK5 and GRK2, respectively.
^{†}5a and 5b are the (R) and (S) enantiomers of Ullrich-57, respectively.

Linker length is important to maintaining GRK5 affinity, as demonstrated by comparison of 5c with 5e and 5d with 5f, wherein the difference is the presence of homologated alkenyl and alkynyl warheads respectively. 5e and 5f demonstrate that as linker length increases, there is a drop-off in GRK5 activity, likely due to a steric clash of the alkenyl or alkynyl warheads with the AST loop or with elements of the large lobe. This indicated that a single methylene linker is key to maintaining GRK5 activity.

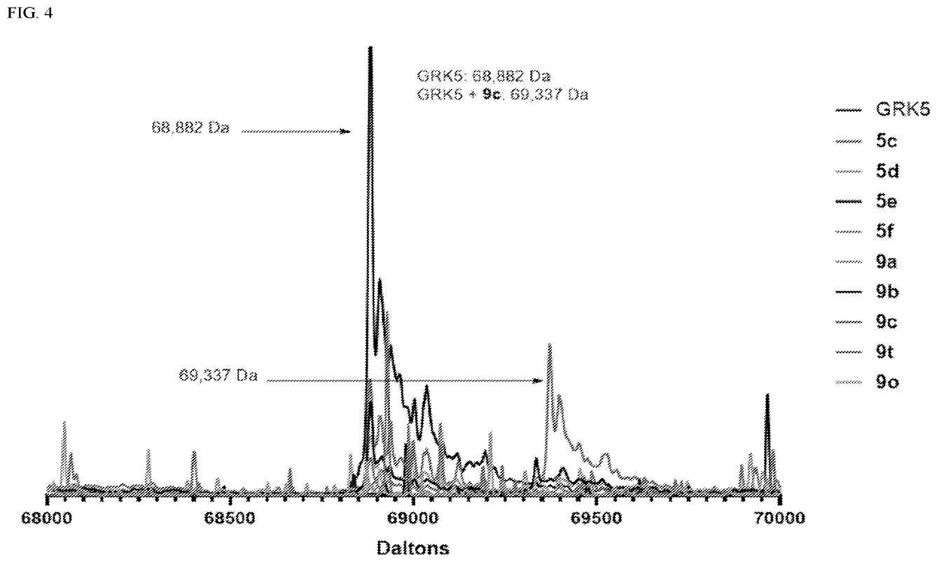

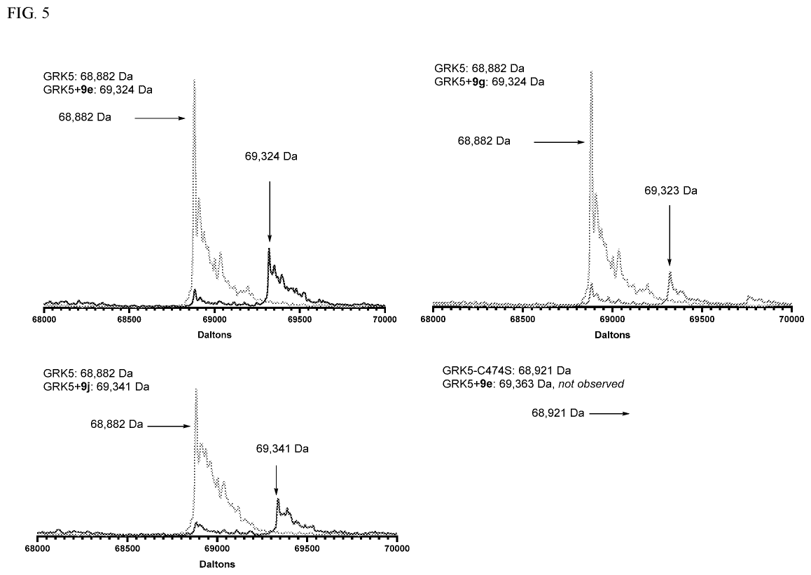

By reversing the amide featured in 5a, the covalent warheads derived from the previous work would become accessible (see, Rowlands, R.; Cato, M. C.; Waldschmidt, H. V.; Bouley, R. A.; Chen, Q.; Avramova, L.; Larsen, S. D.; Tesmer, J. J. G.; White. Structure-Based Design of Selective, Covalent G Protein-Coupled Receptor Kinase 5 Inhibitors|ACS Medicinal Chemistry Letter). To accomplish this, a Knoevenagel with 3,5-dimethyl-4-nitro-1-pyrrolocarbaldehyde yielded a second common intermediate, 7a (Scheme 2). A zinc-catalyzed reduction of the nitro group to the free amine 8a allowed for rapid derivatization through amide coupling to yield final compounds 9a-i. 9a, which features the methylbutynoic acid that could react with Cys474 in a prior study, did not show any covalent engagement when deployed on the indolinone scaffold. It also only exhibited low μM potency against GRK5 (Table 1). This lack of efficacy may be due to the different vector of the warhead off the indolinone scaffold. The commonly used acrylamide and vinyl sulfonamide variants, 9b and 9c, respectively, showed relatively low activity against GRK5, with high nanomolar affinity, yet retained >100 fold selectivity over GRK2. Of these, 9c exhibited covalent modification of GRK5 (FIG. 4). 7a, an intermediate within the synthesis of compounds 9a-t, was tested as it possesses the ability to release nitric oxide (NO) within cells. As NO is a known vasodilator, 7a was considered a possible dual mechanism compound (see, Chen, K.; et al., Antioxid. Redox Signal. 2008, 10 (7), 1185-1198). From an initial examination, it seems that the nitro group is well tolerated (GRK5 IC₅₀=730 nM). However, the 10-fold selectivity over GRK2 is not enough to allow us to determine whether this dual mechanism occurs, specifically in relation to the affect 7a may have on cardiomyocyte contractility.

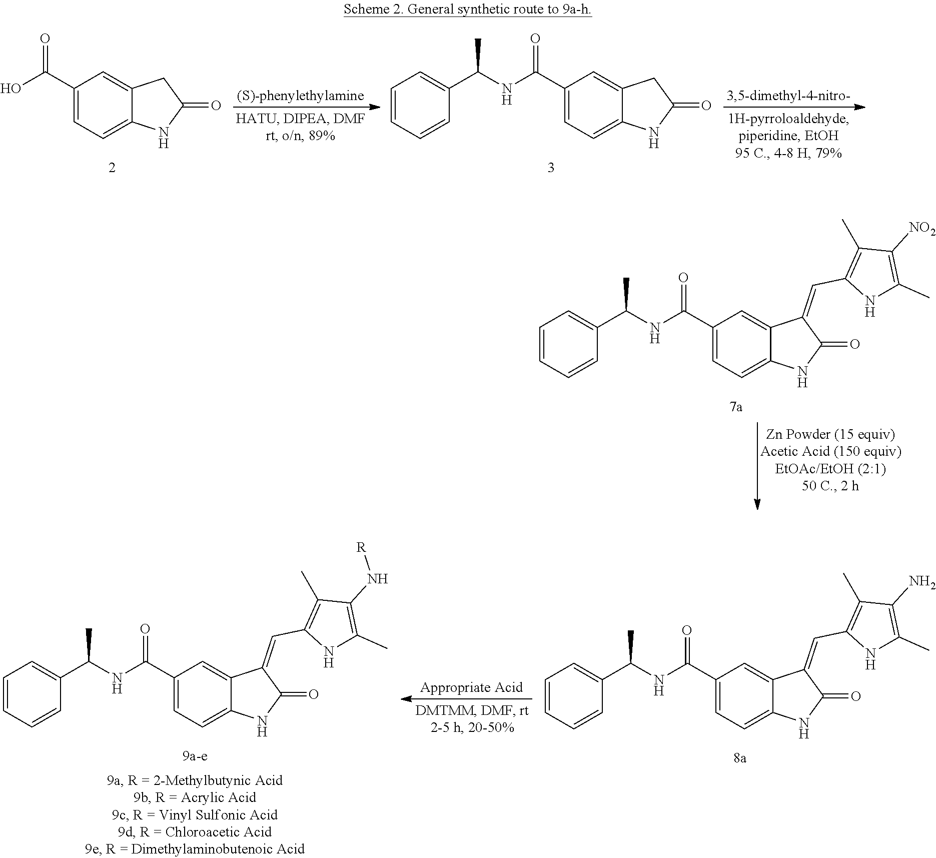

Because the original warheads from previous work were not successful in covalently engaging Cys474, experiments were conducted that chose to investigate more reactive warheads, including the chloroketone featured in 9e (see, Rowlands, R.; Cato, M. C.; Waldschmidt, H. V.; Bouley, R. A.; Chen, Q.; Avramova, L.; Larsen, S. D.; Tesmer, J. J. G.; White. Structure-Based Design of Selective, Covalent G Protein-Coupled Receptor Kinase 5 Inhibitors|ACS Medicinal Chemistry Letters). 9e showed good potency (GRK5 IC₅₀=220 nM) with 1500-fold selectivity over GRK2. That level of selectivity suggests rapid covalent engagement, which was confirmed through covalent MS experiments (FIG. 3). When 9e was tested against GRK5-C474S, covalent interaction was not observed, confirming that Cys474 is the covalent handle for this series (FIG. 5).

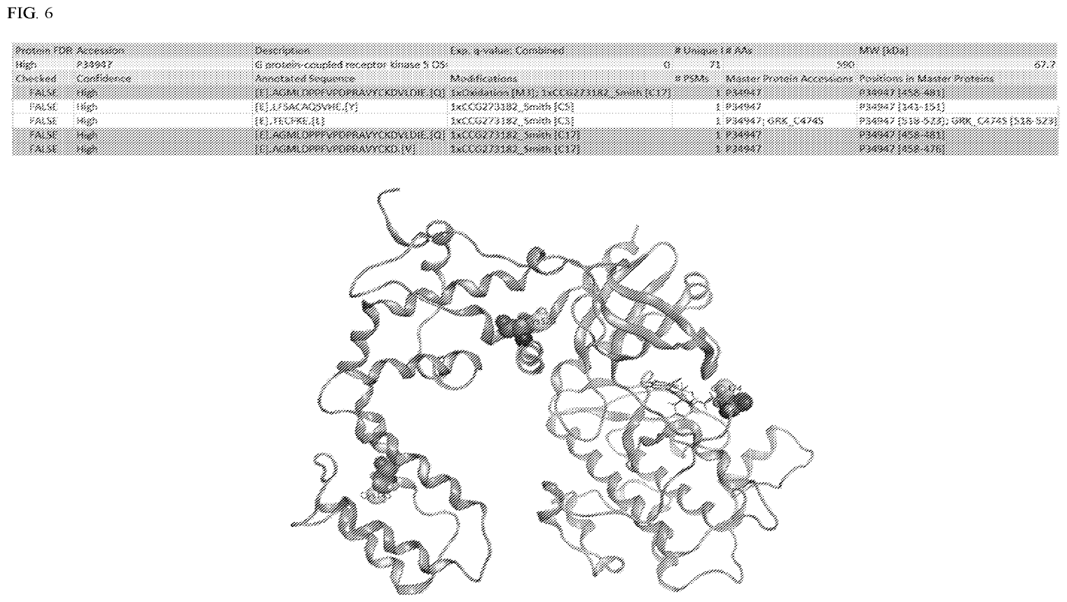

As a final check, 9c was tested in tandem mass spectrometry against GRK5, wherein 9c labeled Cys474 (FIG. 6). Even when tested at lower concentration, 9c still produced some concentration dependent labeling of other cysteines over a 3 h incubation time (see, Rowlands, R.; Cato, M. C.; Waldschmidt, H. V.; Bouley, R. A.; Chen, Q.; Avramova, L.; Larsen, S. D.; Tesmer, J. J. G.; White. Structure-Based Design of Selective, Covalent G Protein-Coupled Receptor Kinase 5 Inhibitors|ACS Medicinal Chemistry Letters; Kim, M.-S.; Zhong, J.; Pandey, A. Common Errors in Mass Spectrometry-Based Analysis of Post-Translational Modifications. Proteomics 2016, 16 (5), 700-714).

Because 5a had low-nanomolar potency, a similar appendage, dimethylaminobutenoic acid, was introduced in 9f. This compound shows an increased IC₅₀ (360 nM, 24-fold higher than 5a), but only 50-fold selectivity over GRK2. 9f did not exhibit covalent attachment by MS (FIG. 3).

The bromoketone compound, 9g was found to have a greater potency against GRK5 (IC₅₀=8.6 nM) than its chloroketone counterpart, 9e. High levels of selectivity against GRK (1400-fold) were maintained. Thus, it was concluded that the bromo group of the bromoketone warhead in 9g must be filling a lipophilic pocket that the chloroketone warhead cannot. It was also observed that 9g took longer to fully engage Cys474 within the 30 min incubation time frame (FIG. 5).

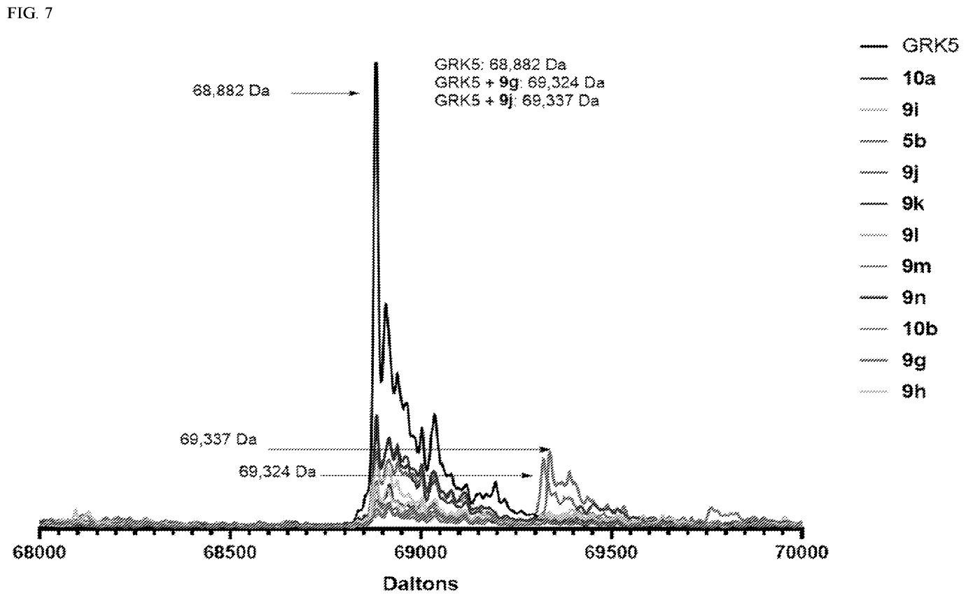

9h was also more potent against GRK5 (IC₅₀=80 nM) than 9e, but it was not found to be covalent (data not shown). Consistent with the trend that was seen in 5e and 5f, it seems clear that homologated linkers do not possess the correct vector for covalent engagement. Similarly, the epoxide warhead, featured in 10a shows a similar level of inability to covalently engage Cys474 (FIG. 7). Given these data, it is clear that the haloketone warheads are the most suitable for a secondary series of GRK5 inhibitors.

Of the warheads explored thus far, only three were able to label GRK5 at Cys474: the haloketones (9e and 9g) and the vinyl sulfonamide (9c). 9c has a 3.4-fold higher IC₅₀ than 9e, suggesting that the vinyl sulfonamide covalent modifier does not satisfy a lipophilic requirement in the area of pocket where it is located. All three compounds had selectivity over GRK2 of ˜400 or greater, whereas the others had a selectivity of ˜100 or lower. The bromoketone warhead of 9g only labelled 50% of GRK5 in the 30 min incubation period, unlike its chloroketone counterpart, 9e (FIG. 7). This is consistent with the fact that bromides are generally less effective leaving groups than chlorides. That said, 9g is still one of the most potent compounds of the series, indicating that the bromine in the warhead is also providing a boost in potency through better packing interactions. However, because of its higher reactivity, the chloroketone warhead was featured in all additional downstream analogs.

Next, SAR around the phenyl pendant was explored (Table 2). GRK5 seems to have a strong preference for para-positioned substituents. Indeed, the meta-substituted or ortho-substituted analogs, 9i, 9l, and 9m were less potent than the para-substituted analogs 9j, 9k and 9o. Additionally, the position of the nitrogen in the pyridyl pendant appeared to make a small difference. In 9k, the potency is 3.5-fold higher than the potency of 9l, suggesting that the para-nitrogen in 9k fulfills some electronic deficiency within the GRK5 binding pocket. 9j, with a 4-fluoro substituent has enhanced potency (IC₅₀=4 nM) relative to the parent compound 9e. Coupled with its ability to form a covalent interaction with Cys474 within 30 min, 9j is thus one of the most intriguing leads of the second series. Interestingly, when the 4-fluoro substituent is maintained, and a bromoketone warhead is used (as seen in 9g) the resulting compound, 9p, shows a slight improvement in potency over 9j (IC₅₀=15 nM). However, 9p is less potent than the des-fluoro compound 9g. This slight loss in potency may be due to a less lipophilic pendant than the benzyl ring featured in 9g.

TABLE 2
Additional Indolinone Variants (μM ± SD)

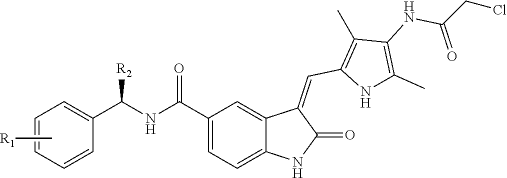

| | | | | | | | | GRK2/ | | |
|---|---|---|---|---|---|---|---|---|---|---|
| Compound | | R₁ | R₂ | GRK5 | | GRK2 | | GRK5^{§} | PKA | |
| CCG | 9i | 3-Me | CH₃ | 0.029 ± 0.03 | (5) | 11 ± 6 | (3) | 360 | >250 | (6) |
| 273261 | | | | | | | | | | |
| CCG | 9j | 4-F | CH₃ | 0.0038 ± 0.001 | (7) | 4.8 ± 3 | (3) | 1300 | >250 | (6) |
| 273441 | | | | | | | | | | |
| CCG | 9k | 4-Py | CH₃ | 0.13 ± 0.05 | (3) | 1.7 ± 2 | (3) | 13 | ND | |
| 273442 | | | | | | | | | | |
| CCG | 9l | 2-Py | CH₃ | 0.45 ± 0.1 | (3) | 9.6 ± 5 | (3) | 21 | ND | |
| 273443 | | | | | | | | | | |
| CCG | 9m | 3-Cl | CH₃ | 0.14 ± 0.04 | (3) | 19 ± 10 | (3) | 140 | >250 | (6) |
| 273444 | | | | | | | | | | |
| CCG | 9n | 4-Me | CH₃ | 0.78 ± 03 | (3) | 2.1 ± 1 | (5) | 3 | ND | |
| 273445 | | | | | | | | | | |
| CCG | 9p | 4-F | CH₃ | 0.015 ± 0.007 | (3) | 3.6 ± 2 | (3) | 230 | ND | |
| 359090^{†} | | | | | | | | | | |
| CCG | 9q | H | — | 0.13 ± 0.09 | (4) | 13 ± 3 | (3) | 99 | >250 | (6) |
| 273561 | | | | | | | | | | |
| CCG 273562 | 9r | H | 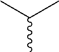 | 12 ± 5 | (3) | 190 ± 70 | (2) | 15 | ND | |
| CCG | 9o | 4-Cl | CH₃ | 0.11 ± 0.05 | (3) | 0.70 ± .2 | (3) | 7 | ND | |
| 273583 | | | | | | | | | | |
| CCG 273564 | 9s | H | 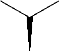 | 0.087 ± 0.02 | (3) | 23 ± 20 | (4) | 260 | >250 | (6) |
| CCG 273563 | 9t | H | 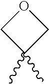 | 0.095 ± 0.03 | (3) | 15 ± 7 | (3) | 160 | 69 ± 10 | (6) |

All data were fit to a log([inhibitor]) versus response model with variable slope and automatic outlier rejection in GraphPad Prism. Curves that had R squared values less than 0.8 after fitting were omitted. ND, not determined. Numbers in parentheses indicate the number of independent experimental curves.
^{†}This compound is the same as 9j but with a bromoketone warhead.

Although GRK5 seems to prefer para positioned pendants, it is clear that the active site cannot accommodate all pendants in that position. In 9n, a 4-methyl pendant drastically reduces potency. However, the 4-chloro pendant seems well tolerated (IC₅₀=110 nM), suggesting that the intolerance for 9n cannot be wholly attributed to steric hinderance.

Given the effects of chirality on activity, as exemplified by 5a and 5b, the benzyl position of this scaffold was further investigated. Interestingly, when the stereocenter is removed, as seen in 9q, there is no loss of potency (IC₅₀=130 nM). However, it is clear from 9s (GRK5 IC₅₀=87 nM) and 9t (GRK5 IC₅₀=95 nM) that having a pendant on the benzyl position has its advantages. This small bump in potency may be due to the stereocenter locking these compounds into low-energy confirmations. When the stereocenter features a more sterically hindered pendant, such as the isopropyl group seen in 9r, the potency for these compounds is almost entirely lost (IC₅₀=12 μM). Therefore, it was concluded that there needs to be a pendant in this position though it must be sufficiently small, and it must have the (R)-configuration to produce optimal binding.

Although the indolinone series offers exceptional potency and selectivity for GRK5, there were a few drawbacks in terms of its pharmacokinetic properties. Firstly, 9a-t are poorly soluble, due to the number of aromatic rings, and the rigid, linear conformation. Solubility concerns were addressed with 9k and 9l, which featured pyridyl pendants known to increase solubility. However, when compared to 9e, 9k was found to have only marginal increases in aqueous solubility (less than 2-fold more soluble; Table 3).

TABLE 3
Thermodynamic Solubility of Indolinone Series Analogues

| | | | | | Dose | |
|---|---|---|---|---|---|---|
| | | | | | Conc. | pH After |
| Compound | | Solution | μM | μg/mL | (mg/mL) | Assay |
| CCG 273220 | 9e | Water | 75.1 | 35.7 | 5.6 | 5 |
| CCG 273221 | 9f | Water | 125 | 64 | 3 | 5 |
| CCG 273441 | 9j | Water | 90.7 | 44.8 | 4 | 3 |
| CCG 273443 | 9l | Water | 262 | 125 | 7.6 | 4 |
| CCG 273445 | 9n | Water | 252 | 124 | 7.6 | 2.5 |
| CCG 273583 | 9o | Water | 205 | 105 | 6 | 4 |

Experimental data collected by Analiza Inc. Experimental values represent an average of n=1 experiments.

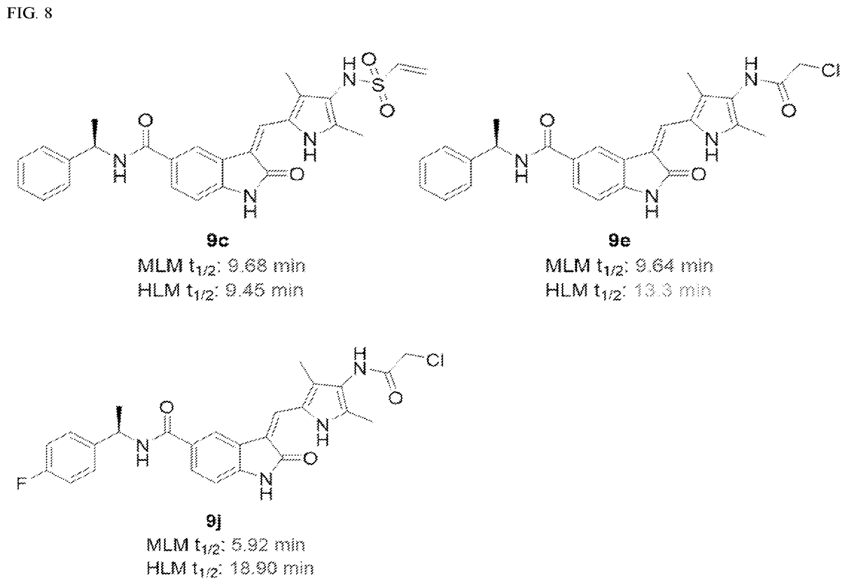

Metabolic liability is also concern because of high lipophilicity, which increases their potential metabolism by CYP3A4. The key positions for metabolism are the phenyl ring and the benzylic position which features a stereocenter. In 9s and 9t, the liability of the benzylic position was addressed through introduction of either an oxetane ring or a geminal dimethyl group, both of which are more stable in metabolic studies than a flag methyl. Pyridyl pendants are known to limit metabolic liability of phenyl rings, as are fluorine pendants, as featured in 9j (FIG. 8). The limited commercial availability of fluorine substituted benzylic amines from the chiral pool limited an ability to explore the additional ortho and meta-substitution patterns. However, as demonstrated by 9k and 9l, with atom-economic changes both aqueous solubility and limiting metabolic liability can be achieved.

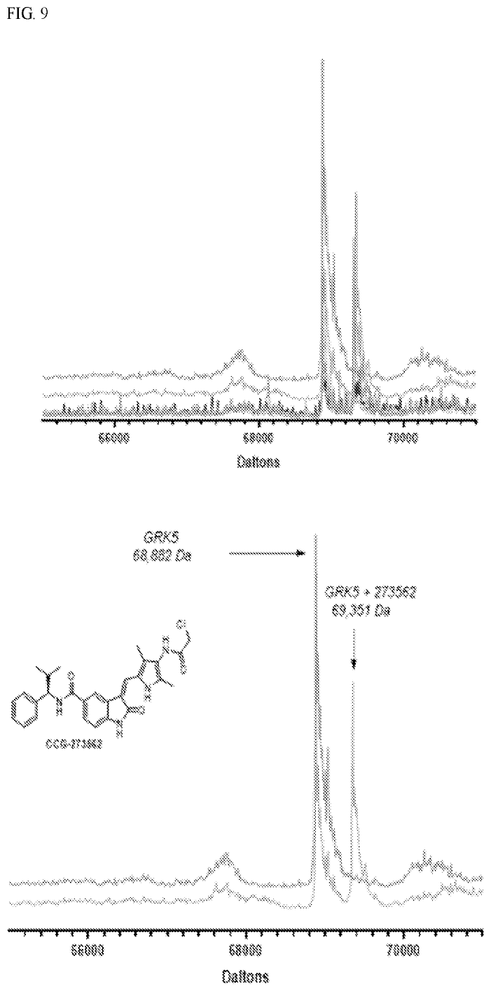

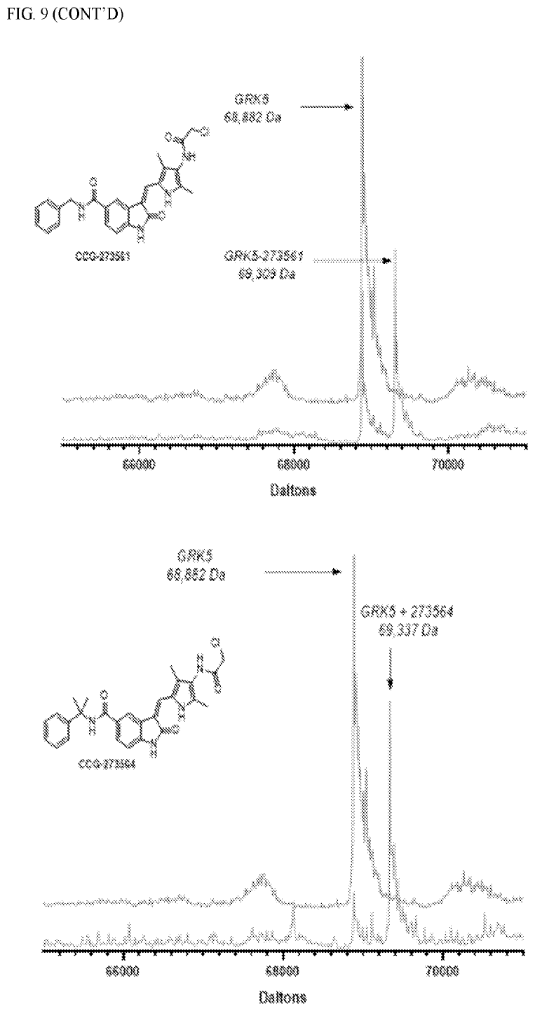

9a-t were further examined in intact protein MS. Interestingly, although all were thought to be able to covalently tag GRK5 because they had chloroketone warheads, not every compound was able to label Cys474 within a 30 min incubation period (FIGS. 4 and 7). This loss in reactivity is most likely due to the higher IC₅₀ in some of these new analogs. Therefore, those compound with lower affinity for GRK5, for example 9c, likely bind to the hinge region more slowly than 9e, resulting in a longer incubation period being needed to detect covalent engagement. When 9a-9s are incubated for 3 h instead of the standard 30 min incubation period, a dramatic increase in covalent interactions is observed, confirming that this effect is kinetic in nature. Indeed, those compounds which do not label after 3 h, were found to label Cys474 after an 8 hr incubation (FIG. 9).

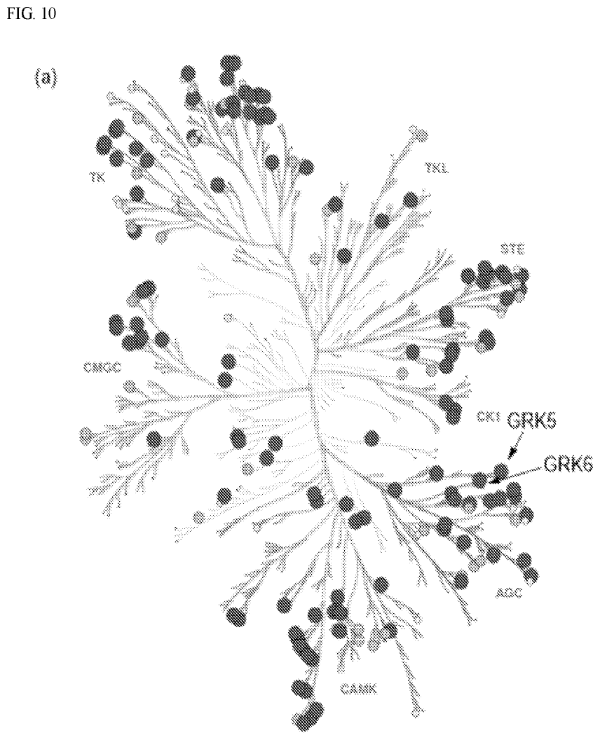

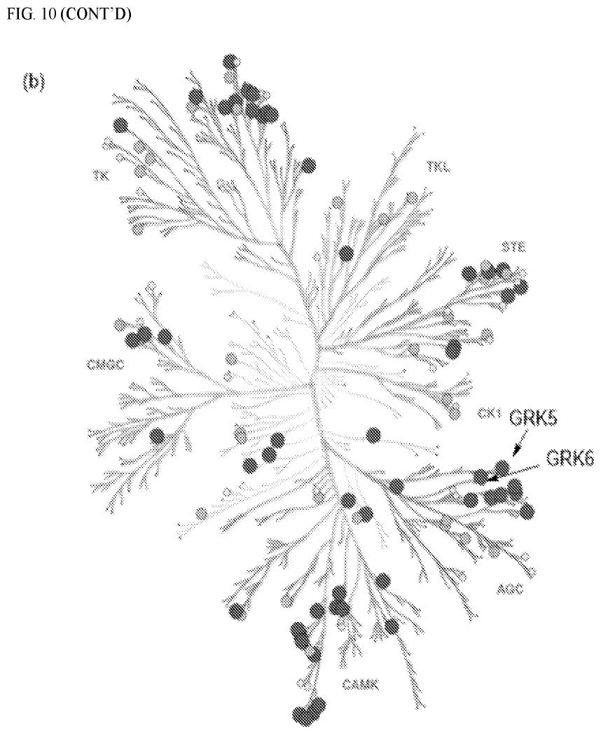

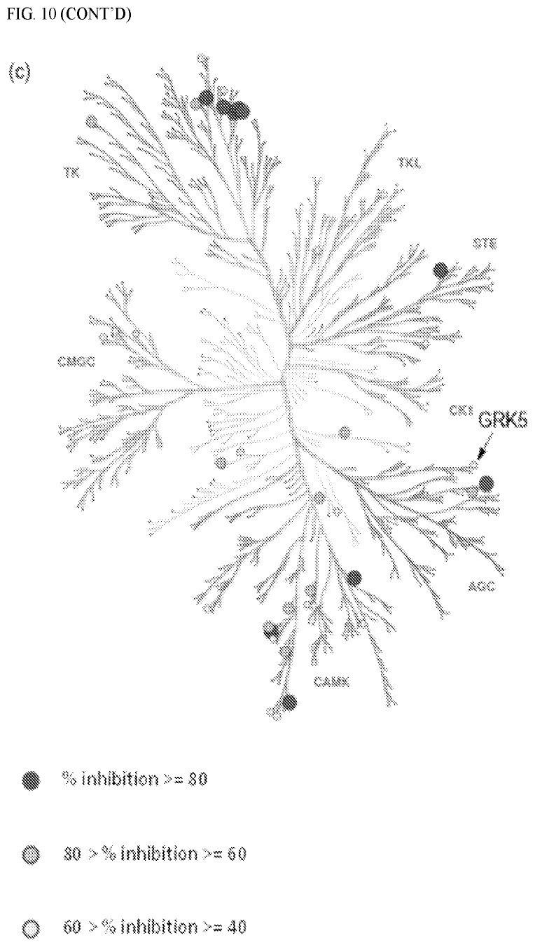

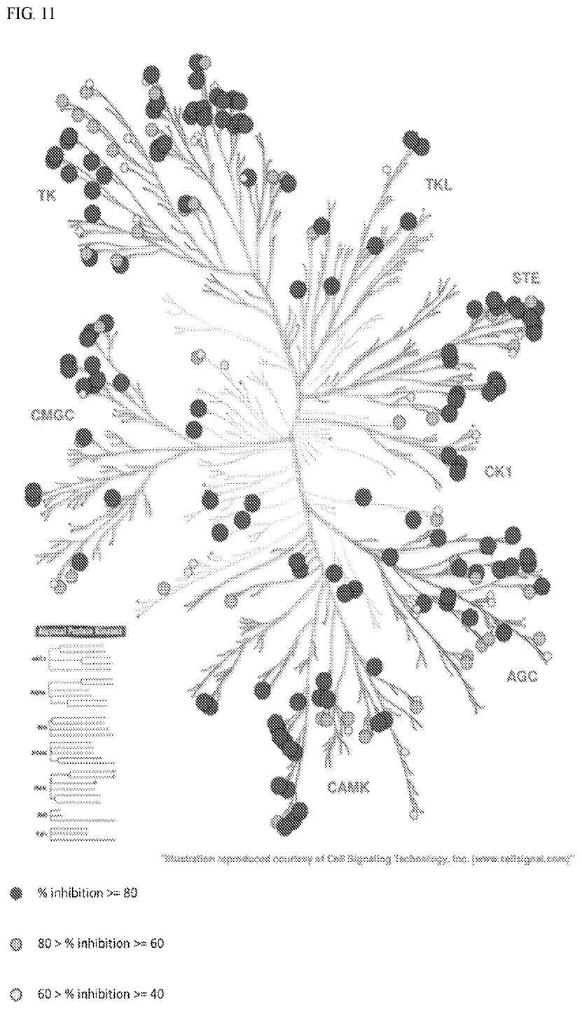

Having confirmed that 5c, and 9g are potent, and highly GRK5 subfamily selective, kinome wide selectivity was explored. In this case, 5c inhibits GRK5 at 92%, and GRK6 at 94% and 9g inhibits GRK5 at 93%, and GRK6 at 97%. When tested at 1 μM, 1000-fold higher than the IC₅₀ for these compounds, it was found that both compounds have many off-target effects across the kinome (FIG. 10 and FIG. 11). These data are not altogether surprising given the fact that the indolinone series is derived from the pan RTK inhibitor, sunitinib, which is believed to act in part by blocking the kinase activity of VEGFR, PDGFR, and c-KIT. In the case of 5c, KIT was 910% inhibited, FGFR1/2 was 92-98% inhibited, PDGFR was 99-100% inhibited and EGFR was 17% inhibited. In the case of 9g, Kit was 84% inhibited, FGFR1/2 was 94-98% inhibited, PDGFR was 99% inhibited and EGFR was 17% inhibited. It was hypothesized that selectivity may improve at lower concentrations of compound, thus 9g was tested at 10 and 100 nM concentrations. Kinome selectivity does depend, in part, on concentration, as 9g becomes far more selective across the kinome when tested at 10 nM, in relation to both the 100 nM and 1 μM panels (FIG. 10). 9g does continue to inhibit a small amount of tyrosine kinases and CAMKs due to the intrinsic activity of the indolinone scaffold itself. In summary, the experiments described herein resulted in the development of a potent series of covalent GRK5 subfamily-selective inhibitors based on an indolinone scaffold. Such experiments further addressed of the metabolic liabilities of this series, and some of the solubility issues. Importantly, the new GRK5 selective compounds set the stage for future cell based and animal studies that will finally allow us to understand the roles of GRK5 versus GRK2 in cardiovascular and other diseases, as well as accelerate the development of GRK5 subfamily selective therapeutics.

### Chemical Synthesis:

General Chemistry: All reagents from commercial sources were used without further purification unless otherwise noted. ¹H-NMR spectra were taken in DMSO-d6, MeOD or CDCl₃ at room temperature on Varian MR 400 MHz, Varian Vnmrs 500 MHz, and Varian Vnmrs 700 MHz instruments. The reported chemical shifts for the ¹HNMR spectra were recorded in parts per million (ppm) on the δ scale from an internal tetramethylsilane (TMS) standard (0.0 ppm). Small molecule mass spectrometry data was measured using a Waters Corporation Micromass LCT or Agilent6230 Q-TOF instrument. HPLC was used to determine purity of compounds on an Agilent 1100 series with an Agilent Zorbax Eclipse Plus-C18 column. A gradient of 10-90% acetonitrile/water over 6 min followed by 90% acetonitrile/water for 7 min was used with detection at 254 nm.

Intact Protein MS and Tandem MS/MS: Intact protein MS was acquired with a Phenomenex C4 column paired with an Agilent 6545 Q-TOF LC/MS. For intact MS and Tandem MS, all samples were prepared with 20 μM GRK in assay buffer (see below), 1 mM compound, and incubated at 4° C. for 3 hr before being quenched with 1.0 μL of formic acid. In Tandem MS/MS, Glu-C was chosen as the restricting enzyme to avoid small fragments with mass-to-charge ratios below the limit of detection of the instrument. All samples were digested with Glu-C sequencing enzyme, procured from Sigma Aldrich (Roche Life Sciences subsidiary) and used without further purification. MS/MS experiments were run on a nano-LC (Dionex RSLC-nano) with an Orbitrap Fusion Tribrid ETD mass spectrometer. This work was conducted by the Proteomics Resource Facility at the University of Michigan.

### Computational Modeling:

GRK5 GRK6 Overlay Model. GRK5 (4WNK) and GRK6 (3NYN) were loaded into Molecular Operating Environment 2018.01 (Molecular Operating Environment (MOE), 2018.01; Chemical Computing Group ULC, 1010 Sherbrooke St. West, Suite #910, Montreal, QC, Canada, H3A 2R7, 2018) and proteins were prepared using QuickPrep function. The sequences of both proteins were aligned and used to create a super-position of the two proteins to create the “GRK5/GRK6 overlay model” which serves as the basis for computational work described throughout this text.

Database Creation. Virtual screening also offers the opportunity to include or exclude compounds known to be frequent “hitters” in high-throughput screens. These frequent “hitters”, known as pan-assay interfering compounds (PAINS) are often excluded from screens for potentially aberrant mechanisms of action, such as aggregation and redox potential. Compounds with known activity for GRK2, GRK5 and ROCK1 (https://www.ebi.ac.uk/chembl/) were downloaded from the Chembl database. These results were then collated with screening compounds from the Maybridge Chemical Library (https://www.maybridge.com/portal/alias__Rainbow/lang__en/tabID__177/DesktopDefault.aspx). The collated library of compounds was then sorted using Pipeline Pilot to remove compounds that contained nitro groups, Michael acceptors, and other chemically reactive moieties, or that may have redox potential (PAINS). The refined list of compounds were imported into MOE, washed and converted from 2D to 3D space. The resultant MDB file, with 2408 compounds, was saved and used in the virtual screening efforts described below.

Pharmacophore Model. For the virtual screen, a pharmacophore was built around the known GRK5 ligand, CCG-215022, as shown in the GRK5 crystal structure (4WNK). The pharmacophore query included the two aromatic rings that form the indazole core of the CCG-215022 scaffold, and the hydrogen bond donor and acceptor from the indazole core. This query was saved and used to determine potential hits within the virtual screening efforts described below.

Virtual Screen. With the screening database and pharmacophore model in hand, the virtual screen was conducted. In the MOE docking protocol, the following parameters were used: Amber10:EHT as the force field, the above-mentioned pharmacophore as the placement, rigid receptor refinement, and London dG, GBVI/WSA dG as the scoring method. All results from the screen were then refined by visual inspection, and compounds with desired binding modes were duplicated for later examination and identification of commercially available sources. As validation of the method, the screen identified Fasudil, a known ROCK1 inhibitor, as a hit. Further visual inspection produced two novel scaffolds, the indolinones and the aminopyridines.

Inhibition Assays: For compounds 5a-9t, IC₅₀ values for human GRK5 and bovine GRK2 were determined using a radiometric assay as follows. 50 nM GRK was incubated with 500 nM porcine brain tubulin (PurSolutions) and 0.01-50 μM inhibitor in 20 mM HEPES pH 7.0, 2 mM MgCl₂, 0.025% dodecylmaltoside (DDM), 1% DMSO, prior to initiation with the addition of 5 μM ATP supplemented with radioactive [□-³²P]-ATP (PerkinElmer Life Sciences). Reactions were quenched at 5 min by addition of 6 μL of 4×SDS gel loading dye to the 6 μL reactions. 8 μL samples were separated on a 4-15% Criterion TGX precast gel (Bio-Rad). For potent inhibitors with low nanomolar IC50, the inhibitor concentration was adjusted to approximately 0-50×[IC50] which was estimated from the first run to get more accurate measurements. Gels were dried, exposed to a storage phosphor screen overnight, and scanned using a Typhoon scanner. Bands corresponding to phosphorylated tubulin were quantified using ImageQuant, plotted as a function of log[inhibitor], and fit to the three-parameter log(inhibitor) vs. response model in GraphPad Prism 7.03 to determine the IC₅₀, and mean and standard deviation values. Outliner was eliminated automatically at 1% Q value. Experiments were performed at least three times.

The PKA inhibition assays were performed with the ADP-Glo system (Promega Corporation) according to the manufactory's instruction. 500 nM of PKA was incubated with 1 μg of CREBtide (KRREILSRRPSYR (SEQ ID NO: 1)) (Genscript Corporation) substrate, 50 μM ATP and inhibitor for 30 min in 20 mM HEPES pH 7.0, 2 mM MgCl₂, 0.025% dodecylmaltoside (DDM), 4% DMSO. The concentration range of each inhibitor varies depending on its solubility at 4% DMSO with the highest concentration from 100 μM to 500 μM. After the initial reaction, ADP-Glo reagent was added to the reaction and allowed to incubate for an additional 40 min. Lastly, the kinase detection reagent was added and allowed to incubate for 30 min, and the luminescence was measured with a FlexStation 3 Multi-mode Microplate Reader (Molecular Devices). All data was analyzed in the same way as GRK inhibition assay. Experiments were performed three times in duplicate.

Standard control compounds are run during each assay to assess consistency across time, experimenters, and subtle changes in assay conditions that are sometimes required to keep some of the compounds soluble and disperse (such as through addition of DDM or 3% DMSO). Paroxetine were used as controls for GRK2 and PKA, and CCG215022 for GRK5.

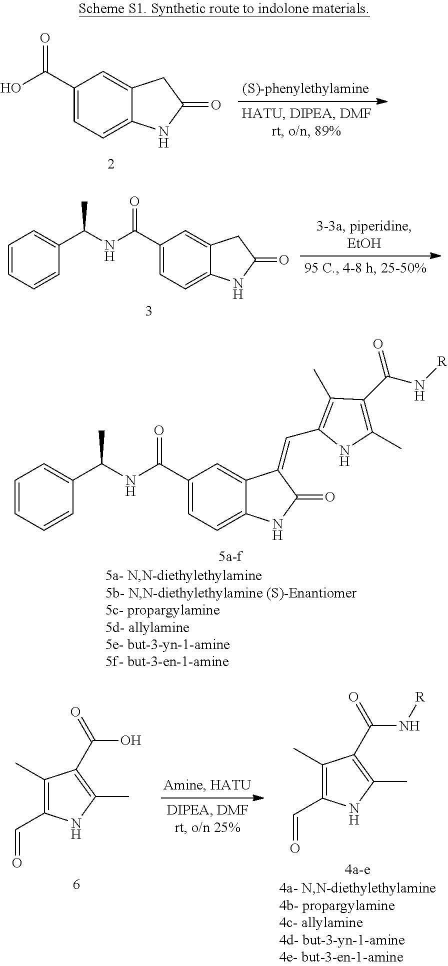

### (R)-2-oxo-N-(1-phenylethyl)indoline-5-carboxamide (3, RAR-11-87)

To a round bottom flask were added 297.7 mg (1.69 mmol) of 2-oxoindoline-5-carboxylic acid, dissolved in 7 mL of dry DMF. To this dark red solution were added 0.244 mL (1.88 mmol) of (S)-1-phenylethan-1-amine, 0.300 mL (1.69 mmol) of DIPEA and 746.1 mg (1.95 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 401.9 mg, 84% Molecular Formula: C₁₇H₁₆N₂O₂ ESI-MS calc: 280.12 ESI-MS found: 281.1283 [M+1] HPLC: 5.198 1H NMR (400 MHz, DMSO-d6) δ 10.62 (s, 1H), 8.63 (d, J=8.0 Hz, 1H), 7.78 (d, J=7.0 Hz, 2H), 7.39-7.35 (m, 2H), 7.31 (dd, J=8.4, 6.8 Hz, 2H), 7.23-7.18 (m, 1H), 6.88-6.83 (m, 1H), 5.15 (p, J=7.2 Hz, 1H), 3.53 (s, 2H), 1.46 (d, J=7.1 Hz, 3H). 13C NMR (100 MHz, dmso) δ 177.17, 165.76, 146.85, 145.65, 129.29, 128.67, 126.52, 126.06, 108.90, 48.85, 36.09, 22.84.

### (S)-2-oxo-N-(1-phenylethyl)indoline-5-carboxamide (3a)

To a round bottom flask were added 201.8 mg (1.13 mmol) of 2-oxoindoline-5-carboxylic acid, dissolved in 7 mL of dry DMF. To this dark red solution were added 0.160 mL (1.24 mmol) of (S)-1-phenylethan-1-amine, 0.200 mL (1.13 mmol) of DIPEA and 492.4 mg (1.30 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 280 mg, 84% Molecular Formula: C₁₇H₁₆N₂O₂ ESI-MS calc: 280.12 ESI-MS found: 281.0903 [M+1] HPLC: 5.249 1H NMR (500 MHz, DMSO-d6) δ 10.67 (s, 1H), 8.66 (d, J=8.0 Hz, 1H), 7.78 (d, J=7.2 Hz, 2H), 7.38 (d, J=8.4 Hz, 2H), 7.31 (t, J=7.6 Hz, 2H), 7.20 (t, J=7.3 Hz, 1H), 6.86 (d, J=8.2 Hz, 1H), 5.15 (p, J=7.2 Hz, 1H), 3.53 (s, 2H), 1.46 (d, J=7.1 Hz, 3H). 13C NMR (176 MHz, dmso) δ 176.83, 165.50, 146.49, 145.22, 128.27, 127.77, 127.54, 126.60, 126.14, 125.64, 123.69, 108.62, 48.52, 39.86, 39.74, 39.62, 39.50, 39.38, 39.26, 39.14, 35.70, 22.40.

### N-(2-(diethylamino)ethyl)-5-formyl-2,4-dimethyl-1H-pyrrole-3-carboxamide (4a, RAR-11-93)

To a round bottom flask were added 200.9 mg (1.20 mmol) of 5-formyl-2,4-dimethyl-1H-pyrrole-3-carboxylic acid, 365.8 mg (1.79 mmol) of EDCI, 277.7 mg (1.79 mmol) of HOBt, 7 mL of dry DMF, 0.200 mL (1.44 mmol) of N1,N1-diethylethane-1,2-diamine and 0.34 mL (2.39 mmol) of TEA. The dark red solution was allowed to stir at rt for 12 h before being quenched with water and extracted with DCM (3×30 mL). The combined organic layer was washed with brine (2×20 mL) and then washed with 10% Citric acid (3×50 mL), drawing the desired product into the water layer. The organic layer was discarded. The aqueous layer was basified with Na2CO3, bringing the pH up to 10, and then extracted with DCM (4×50 mL). The combined organic layers were dried over MgSO₄ and then evaporated onto silica gel and purified by column chromatography (5-15% MeOH/DCM). The solvent was removed under pressure to give a yellow solid. Result: light yellow solid, 90 mg, 27% Molecular Formula: C₁₄H₂₃N₃O₂, ESI-MS Calc: 265.18 ESI-MS found: 266.1749 HPLC: 2.681 1H NMR (400 MHz, DMSO-d6) δ 11.85 (s, 1H), 9.54 (s, 1H), 7.95 (s, 2H), 2.34 (d, J=20.8 Hz, 7H), 0.99 (s, 6H).

### 5-formyl-2,4-dimethyl-N-(prop-2-yn-1-yl)-1H-pyrrole-3-carboxamide (4b, RAR-11-97)

To a round bottom flask were added 199.4 mg (1.20 mmol) of 5-formyl-2,4-dimethyl-1H-pyrrole-3-carboxylic acid, 688.0 mg (1.79 mmol) of HATU, 6 mL of dry DMF, 0.100 mL (1.44 mmol) of propargylamine and 0.34 mL (2.39 mmol) of TEA. The dark red solution was allowed to stir at rt for 12 h before being quenched with water and extracted with DCM (3×30 mL). Took the combined organic layers and washed with brine (2×20 mL) and then washed with 10% Citric acid (3×50 mL), taking the desired product into the aqueous layer. Discarded the organic layer. The aqueous layer was basified with Na2CO3, bringing the pH up to 10, and then extracted with DCM (4×50 mL). The combined organic layer was dried over over MgSO₄ and then removed solvent to give the final product as a light orange oil. Result: light orange oil, 61 mg, 25% Molecular Formula: C₁₁H₁₂N₂O₂ ESI-MS calc: 204.09 ESI-MS found: 205.0873 [M+1], 242.1167 [M+K] HPLC: 3.625 1H NMR (700 MHz, DMSO-d6) δ 11.87 (s, 1H), 9.55 (s, 1H), 7.95 (s, 16H), 3.97 (ddd, J=12.7, 5.7, 2.5 Hz, 3H), 3.08 (t, J=2.4 Hz, 1H), 2.36 (s, 3H), 2.31 (s, 3H). 13C NMR (176 MHz, dmso) δ 177.82, 164.77, 139.11, 138.51, 128.26, 119.20, 89.92, 82.16, 72.95, 55.38, 40.24, 38.71, 28.42, 12.90, 10.00.

### N-allyl-5-formyl-2,4-dimethyl-1H-pyrrole-3-carboxamide (4c, RAR-11-99)

To a round bottom flask were added 204.4 mg (1.20 mmol) of 5-formyl-2,4-dimethyl-1H-pyrrole-3-carboxylic acid, 350.3 mg of EDCI (1.79 mmol), 275.3 mg (1.79 mmol) of HOBt, 6 mL of dry DMF, 0.110 mL (1.44 mmol) of allylamine and 0.34 mL (2.39 mmol) of TEA. The dark red solution was allowed to stir at rt for 12 h before being quenched with water and extracted with DCM (3×30 mL). Took combined organic layers and washed with LiCl 3×20 mL) and then washed with 10% Citric acid (3×50 mL), drawing the desired product into the aqueous layer. Discarded the organic layer. The aqueous layer was basified with Na2CO3, bringing the pH up to 8, and then extracted with DCM (4×50 mL) The combined organic layer was dried over MgSO₄ and then removed solvent to give a yellow solid. Result: light yellow solid, 64 mg, 25% Molecular Formula: C₁₁H₁₄N₂O₂ ESI-MS calc: 206.11 ESI-MS: 246.1621 [M+MeCN] HPLC: 3.885 ¹H NMR (500 MHz, DMSO-d6) δ 11.38 (s, 1H), 9.54 (s, 1H), 8.17 (s, 1H), 6.01 (tt, J=10.8, 5.4 Hz, 1H), 5.87 (ddt, J=16.4, 10.6, 5.3 Hz, 2H), 5.18 (t, J=15.7 Hz, 3H), 5.08 (dd, J=16.5, 10.2 Hz, 3H), 4.14 (d, J=5.5 Hz, 2H), 3.82 (t, J=5.7 Hz, 3H), 2.31 (s, 5H), 2.23 (s, 3H). ¹³C NMR (126 MHz, dmso) δ 161.78, 150.29, 135.45, 135.21, 115.01, 114.35, 114.20, 40.54, 40.50, 39.50, 39.33, 39.17, 39.00, 38.83, 38.67, 38.50, 35.26, 30.25, 11.95, 9.27.

### N-(but-3-yn-1-yl)-5-formyl-2,4-dimethyl-1H-pyrrole-3-carboxamide (4d, RAR-12-2)

To a round bottom flask were added 199.6 mg (1.20 mmol) of 5-formyl-2,4-dimethyl-1H-pyrrole-3-carboxylic acid, 684.3 mg (1.79 mmol) of HATU, 6 mL of dry DMF, 0.100 mL (1.44 mmol) of 1-amino-3-butyne and 0.30 mL (2.39 mmol) of DIPEA. The dark red solution was allowed to stir at rt for 12 h before being quenched with water and extracted with DCM (3×30 mL). The combined organic layers were washed with LiCl (2×20 mL) and then washed with 10% Citric acid (3×50 mL), drawing the desired product into the water layer. Discarded the organic layer. The aqueous layer was basified with Na2CO3, bringing the pH up to 10, and then extracted with DCM (4×50 mL). The combined organic layer was dried over MgSO₄ and then removed solvent under pressure to give the final product as a yellow solid. Result: light yellow solid, 99.6 mg, 38.1% Molecular Formula: C₁₂H₁₄N₂O₂ ESI-MS calc: 218.11 ESI-MS found: 219.1129 HPLC: 3.997 1H NMR (700 MHz, DMSO-d6) δ 11.83 (s, 1H), 2.83 (s, 1H), 2.69 (d, J=1.0 Hz, 1H), 2.60 (d, J=2.4 Hz, 3H), 2.37 (s, 4H), 2.32 (s, 3H). 13C NMR (176 MHz, dmso) δ 178.94, 164.54, 160.23, 152.56, 147.04, 146.58, 126.55, 115.31, 88.48, 82.43, 72.02, 55.77, 37.85, 18.85, 13.97, 10.55.

### N-(but-3-en-1-yl)-5-formyl-2,4-dimethyl-1H-pyrrole-3-carboxamide (4e, RAR-12-3)

To a round bottom flask were added 197.8 mg (1.20 mmol) of 5-formyl-2,4-dimethyl-1H-pyrrole-3-carboxylic acid, 685.8 mg (1.79 mmol) of HATU, 6 mL of dry DMF, 107.1 mg (1.44 mmol) of but-3-en-1-amine and 0.45 mL (2.40 mmol) of DIPEA. The dark red solution was allowed to stir at rt for 12 h before being quenched with water and extracted with DCM (3×30 mL). The combined organic layers were washed with brine (2×20 mL) and then washed with 10% Citric acid (3×50 mL), drawing the desired material in to the aqueous layer. Discarded the organic layer. The aqueous layer was basified with Na2CO3, bringing the pH up to 10, and then extracted with DCM (4×50 mL). The combined organic layer was dried over MgSO₄ and then removed solvent. Result: light orange oil, 106.1 mg, 37.2% Molecular Formula: C₁₂H₁₆N₂O₂ ESI-MS calc: 220.12 MS: 221.1304 HPLC: 5.554 1H NMR (500 MHz, DMSO-d6) δ 9.77 (s, 1H), 8.84 (d, J=4.5 Hz, 1H), 8.73 (d, J=8.4 Hz, 1H), 7.95 (s, 4H), 7.66 (dd, J=8.7, 4.6 Hz, 1H), 5.12-4.99 (m, 1H), 2.59 (s, 7H). 13C NMR (126 MHz, dmso) δ 179.37, 165.02, 162.73, 152.99, 146.27, 140.66, 134.88, 130.23, 122.09, 36.21, 31.20, 14.39, 10.98.

### (R,Z)-3-((4-((2-(diethylamino)ethyl)carbamoyl)-3,5-dimethyl-1H-pyrrol-2-yl)methylene)-2-oxo-N-(1-phenylethyl)indoline-5-carboxamide (5a,RAR-11-96)

To a dried sealed tube were added 97.9 mg (0.371 mmol) of (3), 107.0 mg (0.357 mmol) of (4a) all of which were dissolved in abs. EtOH (3.5 mL). To this solution were added 2 drops of piperidine and heated to reflux (95° C.) for 4 h. Once complete, cooled to room temperature and then filtered off the product as an orange solid. Yield: orange solid, 38.3 mg, 22% Molecular Formula: C₃₁H₃₇N₅O₃ ESI-MS calc: 527.29 ESI-MS found: 528.2955 HPLC: 5.621 1HNMR (500 MHz, DMSO-d6) δ 13.59 (s, 1H), 11.13 (s, 1H), 8.59 (d, J=8.1 Hz, 1H), 8.25 (s, 1H), 7.71 (d, J=6.8 Hz, 2H), 7.44 (d, J=6.0 Hz, 1H), 7.41 (d, J=7.9 Hz, 2H), 7.33 (t, J=7.7 Hz, 2H), 7.22 (t, J=7.7 Hz, 1H), 6.93 (d, J=8.3 Hz, 1H), 5.19 (t, J=7.5 Hz, 1H), 3.28 (d, J=7.0 Hz, 2H), 2.44 (d, J=6.7 Hz, 6H), 1.51 (d, J=7.1 Hz, 3H), 0.97 (t, J=7.1 Hz, 7H). 13CNMR (176 MHz, dmso) δ 169.90, 165.95, 164.71, 145.11, 140.64, 136.48, 129.93, 128.30, 127.78, 126.64, 126.37, 126.18, 125.86, 125.28, 124.09, 120.76, 117.69, 114.37, 108.94, 51.72, 48.55, 46.59, 45.44, 39.86, 39.74, 39.62, 39.50, 39.38, 39.26, 39.14, 37.06, 22.35, 13.40, 11.92, 10.79.

### (S,Z)-3-((4-((2-(diethylamino)ethyl)carbamoyl)-3,5-dimethyl-1H-pyrrol-2-yl)methylene)-2-oxo-N-(1-phenylethyl)indoline-5-carboxamide (5b)

To a dried sealed tube were added 91.2 mg (0.325 mmol) of (3a), 79.9 mg (0.299 mmol) of (4a) all of which were dissolved in abs. EtOH (2.5 mL). To this solution were added 2 drops of piperidine and heated to reflux (95° C.) for 4 h. Once complete, cooled to room temperature and then filtered off the product as an orange solid. Yield: orange solid, 38.3 mg, 22% Molecular Formula: C₃₁H₃₇N₅O₃ ESI-MS calc: 527.29 ESI MS found: 528.2043 HPLC: 5.753 1H NMR (500 MHz, DMSO-d6) δ 13.59 (s, 1H), 11.16 (s, 1H), 8.67 (s, 1H), 8.32 (d, J=5.5 Hz, 1H), 7.75-7.68 (m, 2H), 7.48 (s, 1H), 7.42 (d, J=7.7 Hz, 3H), 7.32 (t, J=7.6 Hz, 3H), 7.22 (t, J=7.3 Hz, 1H), 6.94 (d, J=8.1 Hz, 1H), 5.19 (p, J=7.2 Hz, 1H), 2.45 (d, J=3.4 Hz, 8H), 2.37 (s, 1H), 2.32 (s, 1H), 2.08 (s, 1H), 1.51 (d, J=7.1 Hz, 4H), 0.99 (t, J=7.5 Hz, 8H). 13C NMR (126 MHz, dmso) δ 170.28, 147.82, 141.01, 130.32, 128.66, 127.23, 126.98, 126.61, 126.26, 125.66, 124.60, 118.30, 106.84, 102.36, 48.92, 47.02, 22.80, 17.46, 13.83, 11.24, 9.18.

### (R,Z)-3-((3,5-dimethyl-4-(prop-2-yn-1-ylcarbamoyl)-1H-pyrrol-2-yl)methylene)-2-oxo-N-(1-phenylethyl)indoline-5-carboxamide (5c,RAR-11-98)

To a dried sealed tube were added 75.1 mg (0.27 mmol) of (3), 50 mg (0.28 mmol) of (4b) all of which were dissolved in abs. EtOH (1.8 mL). To this solution were added 0.05 mL of piperidine and heated to reflux (95° C.) for 4 h. Once complete, cooled to room temperature and filtered off an orange solid. The solid was rinsed with cold EtOH to give the final product. Yield: bright orange solid, 58.6 mg, 46% Molecular Formula: C₂₈H₂₆N₄O₃ ESI-MS calc: 466.20 ESI-MS found: 467.2037 HPLC: 6.360 1H NMR (700 MHz, DMSO-d6) δ 13.61 (s, 1H), 11.15 (s, 1H), 8.61 (d, J=8.0 Hz, 1H), 8.26 (s, 1H), 8.06 (t, J=5.5 Hz, 1H), 7.71 (d, J=9.0 Hz, 2H), 7.41 (d, J=7.8 Hz, 2H), 7.33 (q, J=5.8, 3.9 Hz, 2H), 7.23 (t, J=7.2 Hz, 1H), 6.94 (d, J=8.2 Hz, 1H), 5.20 (t, J=7.7 Hz, 1H), 4.02 (d, J=5.5 Hz, 2H), 3.14-3.08 (m, 1H), 2.44 (d, J=9.2 Hz, 7H), 1.51 (d, J=7.1 Hz, 3H). 13C NMR (176 MHz, dmso) δ 170.30, 166.26, 145.52, 141.06, 136.92, 130.46, 128.67, 128.29, 127.00, 126.82, 126.58, 126.28, 124.49, 120.45, 118.23, 115.03, 109.28, 82.17, 73.04, 48.87, 28.49, 22.77, 13.76, 11.13.

### (R,Z)-3-((4-(allylcarbamoyl)-3,5-dimethyl-1H-pyrrol-2-yl)methylene)-2-oxo-N-(1-phenylethyl)indoline-5-carboxamide (5d, RAR-11-100)

To a dried sealed tube were added 53.6 mg (0.27 mmol) of (3), 50 mg (0.28 mmol) of (4c) all of which were dissolved in abs. EtOH (3 mL). To this solution were added 2 drops (0.05 mL) of piperidine and heated to reflux (95° C.) for 4 h. Once complete, the reaction was cooled to room temperature and then an orange solid was filtered off. The solid was washed with cold EtOH to give the final product. Result: orange solid, 79.5 mg, 63% Molecular Formula: C₂₈H₂₈N₄O₃ ESI-MS calc: 468.22 ESI-MS found: 469.2224 HPLC: 6.450 1H NMR (700 MHz, DMSO-d6) δ 13.60 (s, 1H), 11.14 (s, 1H), 8.60 (d, J=8.0 Hz, 1H), 8.25 (d, J=1.6 Hz, 1H), 7.83 (t, J=5.8 Hz, 1H), 7.73-7.68 (m, 2H), 7.41 (d, J=7.6 Hz, 2H), 7.33 (t, J=7.6 Hz, 2H), 7.22 (t, J=7.3 Hz, 1H), 6.94 (d, J=8.1 Hz, 1H), 5.90 (ddt, J=15.7, 10.3, 5.2 Hz, 1H), 5.22-5.18 (m, 2H), 5.10 (dd, J=10.3, 1.8 Hz, 1H), 3.87 (t, J=5.6 Hz, 2H), 2.44 (d, J=7.8 Hz, 6H), 1.51 (d, J=7.1 Hz, 3H). 13C NMR (176 MHz, dmso) δ 170.50, 145.29, 135.97, 130.84, 129.65, 128.44, 128.02, 126.34, 115.20, 108.42, 103.80, 91.16, 59.93, 22.53, 13.55, 10.93.

### (R,Z)-3-((4-(but-3-yn-1-ylcarbamoyl)-3,5-dimethyl-JH-pyrrol-2-yl)methylene)-2-oxo-N-(1-phenylethyl)indoline-5-carboxamide (5e, RAR-12-4)

To a dried sealed tube were added 99.9 mg (0.357 mmol) of (3), 82 mg of RAR-12-2 (4d) all of which were dissolved in abs. EtOH (3.5 mL). To this solution were added 2 drops (0.05 mL) of piperidine and heated to reflux (95° C.) for 4 h. Once complete, the solution was cooled to room temperature and then an orange solid was filtered off. The solid was washed with cold EtOH to give the final product. Yield: orange solid, 19 mg, 10% Molecular Formula: C₂₉H₂₈N₄O₃ ESI-MS calc: 480.22 ESI-MS found: 481.2226 HPLC: 6.444 1H NMR (700 MHz, DMSO-d6) δ 13.60 (s, 1H), 11.15 (s, 1H), 8.62 (d, J=8.0 Hz, 1H), 8.25 (s, 1H), 7.79 (t, J=5.8 Hz, 1H), 7.71 (d, J=6.3 Hz, 2H), 7.41 (d, J=7.6 Hz, 2H), 7.33 (t, J=7.6 Hz, 2H), 7.23 (t, J=7.3 Hz, 1H), 6.94 (d, J=8.2 Hz, 1H), 5.19 (p, J=7.3 Hz, 1H), 2.86 (t, J=2.5 Hz, 1H), 2.45 (d, J=12.0 Hz, 6H), 2.42 (td, J=7.1, 2.4 Hz, 2H), 1.51 (d, J=7.0 Hz, 3H). 13C NMR (176 MHz, dmso) δ 169.83, 165.81, 164.71, 145.08, 140.56, 136.34, 130.00, 128.23, 127.80, 126.55, 126.12, 125.79, 125.18, 124.08, 120.61, 117.76, 82.50, 72.12, 48.42, 37.95, 22.33, 13.33, 10.70.

### (R,Z)-3-((4-(but-3-en-1-ylcarbamoyl)-3,5-dimethyl-1H-pyrrol-2-yl)methylene)-2-oxo-N-(1-phenylethyl)indoline-5-carboxamide (5f RAR-12-5)

To a dried sealed tube were added 94.8 mg (0.27 mmol) of (3), 87.2 mg (0.28 mmol) of RAR-12-3 (4e) all of which were dissolved in abs. EtOH (2.2 mL). To this solution were added 2 drops (0.07 mL) of piperidine and heated to reflux (95° C.) for 4 h. Once complete, solution was cooled to room temperature and then purified by column chromatography. Fractions were collected and solvent was removed under pressure to give the desired product, as an orange solid. Yield: orange solid, 103 mg, 59% Molecular Formula: C₂₉H₃₀N₄O₃ ESI-MS calc: 482.23 ESI-MS found: 483.2382 [M+1] HPLC: 6.720 1H NMR (700 MHz, DMSO-d6) δ 13.54 (s, 1H), 11.13 (s, 1H), 8.61 (d, J=8.2 Hz, 2H), 8.27-8.18 (m, 2H), 7.73-7.71 (m, 1H), 7.68 (s, 1H), 7.42 (d, J=8.3 Hz, 3H), 7.35-7.31 (m, 4H), 7.25-7.21 (m, 2H), 6.94 (d, J=8.1 Hz, 1H), 5.21 (p, J=7.3 Hz, 2H), 2.43 (d, J=9.0 Hz, 2H), 2.29 (d, J=11.4 Hz, 7H), 1.63-1.59 (m, 3H), 1.51 (d, J=7.1 Hz, 6H). 13C NMR (176 MHz, dmso) δ 169.79, 165.81, 165.04, 145.07, 140.57, 133.90, 128.74, 128.19, 127.78, 126.52, 126.11, 125.22, 123.96, 120.71, 117.70, 116.21, 114.09, 108.74, 48.41, 35.77, 33.74, 22.26, 12.40, 10.15.

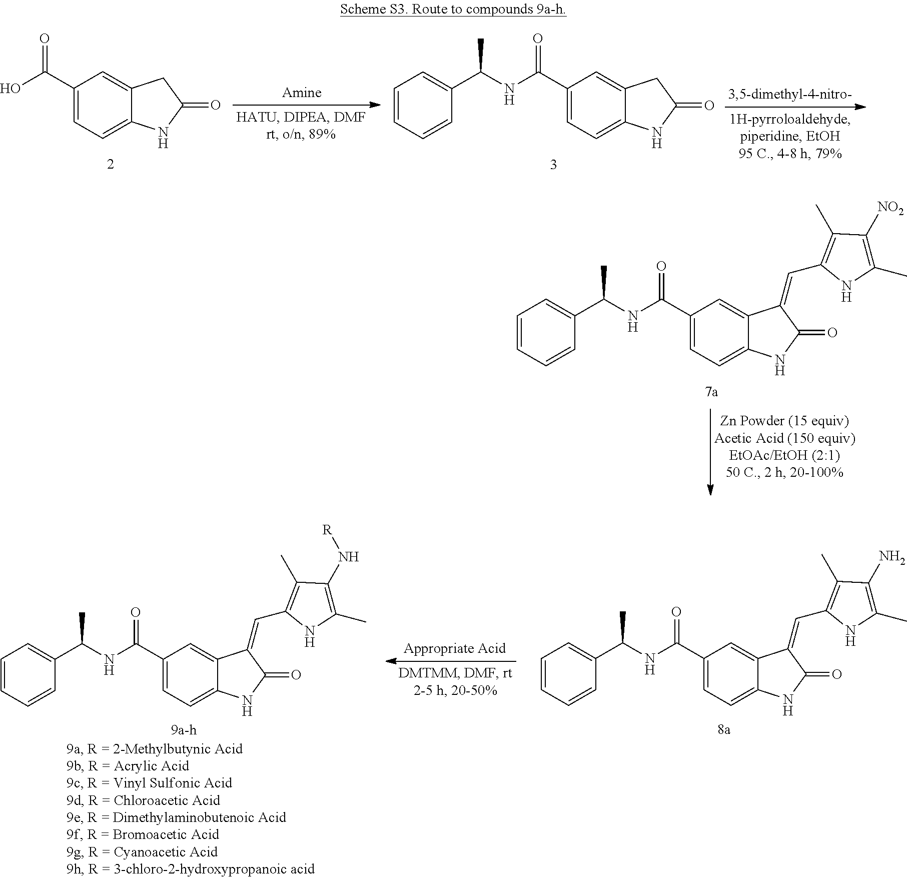

### (R,Z)-3-((3,5-dimethyl-4-nitro-1H-pyrrol-2-yl)methylene)-2-oxo-N-(1-phenylethyl)indoline-5-carboxamide (7a, RAR-12-6)

To a dried sealed tube were added 91.2 mg (0.892 mmol) of (3), 79.9 mg (1.39 mmol) of 3,5-dimethyl-4-nitro-1H-pyrrole-2-carbaldehyde, all of which were dissolved in abs. EtOH (2.5 mL). To this solution were added 2 drops (0.05 mL) of piperidine and heated to reflux (95° C.) for 4 h. Once complete, the reaction was cooled to room temperature and an orange solid was filtered off. The solid was washed with cold EtOH to give the desired product. Result: orange solid, 269.3 mg, 69% Molecular Formula: C₂₄H₂₂N₄O₄ ESI-MS calc: 430.16 ESI-MS found: 431.1715 HPLC: 7.400 1H NMR (700 MHz, DMSO-d6) δ 8.63 (d, J=8.0 Hz, 1H), 8.36 (d, J=1.6 Hz, 1H), 7.83 (s, 1H), 7.76 (dd, J=8.1, 1.7 Hz, 1H), 7.41 (d, J=7.5 Hz, 2H), 7.33 (t, J=7.6 Hz, 2H), 7.22 (t, J=7.3 Hz, 1H), 6.97 (d, J=8.1 Hz, 1H), 5.19 (p, J=7.2 Hz, 1H), 2.08 (d, J=1.0 Hz, 1H), 1.50 (d, J=7.1 Hz, 3H). 13C NMR (176 MHz, dmso) δ 169.90, 165.69, 144.98, 142.47, 136.80, 132.12, 130.48, 129.11, 128.20, 127.74, 126.10, 125.56, 123.33, 121.76, 115.38, 53.66, 22.28, 18.23, 10.50.

### (R,Z)-3-((4-amino-3,5-dimethyl-1H-pyrrol-2-yl)methylene)-2-oxo-N-(1-phenylethyl)indoline-5-carboxamide (8a, RAR-12-7)

To a flask were added 90.7 mg of (7a), dissolved in 5 mL of 2:1 EtOH/EtOAc. To this slurry were added 226.8 mg (14 equiv) of Zn powder and 2 mL (150 equiv) of AcOH. The turbid orange solution was allowed to stir at 50 C for 2 h. Once complete, the reaction was cooled to rt and then add EtOAc before basifying with sat. Na2CO3. The basified aqueous layer was extracted with EtOAc (3×30 mL) and then washed with water and brine (1×30 mL), respectively. The organic layer was dried over MgSO₄ and then solvent was removed under pressure to give the desired product as a red solid. *Note: the free amine is very reactive, so it was moved forward without further characterization* Result: orange solid, 66.7 mg, 79.0% Molecular Formula: C₂₄H₂₄N₄O₂ ESI-MS calc: 400.19 ESI-MS found: 401.1955 HPLC: 5.177 1H NMR (700 MHz, DMSO-d6) δ 13.45 (s, 1H), 10.88 (s, 1H), 8.56 (d, J=8.0 Hz, 1H), 8.12 (d, J=1.6 Hz, 1H), 7.62 (dd, J=8.1, 1.6 Hz, 1H), 7.46 (s, 1H), 7.41 (d, J=7.6 Hz, 3H), 7.33 (t, J=7.6 Hz, 3H), 7.22 (t, J=7.3 Hz, 1H), 6.89 (d, J=8.1 Hz, 1H), 5.20 (q, J=7.4 Hz, 1H), 4.02 (d, J=14.5 Hz, 2H), 2.26 (s, 3H), 2.17 (s, 3H), 1.51 (d, J=7.1 Hz, 3H).

### (R,Z)-3-((4-(but-2-ynamido)-3,5-dimethyl-1H-pyrrol-2-yl)methylene)-2-oxo-N-(1-phenylethyl)indoline-5-carboxamide (9a, RAR-12-17)

To a flask were added 22.3 mg (0.37 mmol) of butynoic acid, 157.5 mg (0.37 mmol) of HATU, cat. DMAP and 50 mg (0.19 mmol) of 8a dissolved in 1 mL of DMF. To this bright red solution were added 0.25 mL (1.3 mmol) of DIPEA, and the solution was allowed to stir at rt for 2 h. Once complete, the reaction mixture was diluted with EtOAc and washed with sat. LiCl and then dried over MgSO₄. The crude material was purified by prepatory TLC with 50% Acetone/Hexanes. The desired band was collected, and the material was rinsed off the silica gel with acetone and then solvent was removed under pressure to give the desired product. Result: yellow solid, 17.5 mg, 20% Molecular Formula: C₂₈H₂₆N₄O₃ ESI-MS calc: 466.20 ESI-MS found 467.2071 HPLC: 6.336 ¹H NMR (700 MHz, DMSO-d6) δ 13.53 (s, 1H), 11.07 (s, 1H), 9.78 (s, 1H), 8.86 (d, J=4.5 Hz, 2H), 8.68 (d, J=8.8 Hz, 2H), 8.60 (d, J=7.7 Hz, 2H), 8.21 (s, 1H), 7.69 (d, J=8.9 Hz, 2H), 7.65-7.62 (m, 3H), 7.41 (d, J=7.9 Hz, 5H), 7.33 (t, J=7.3 Hz, 7H), 7.23 (d, J=7.0 Hz, 3H), 6.93 (d, J=8.1 Hz, 1H), 6.59 (s, 3H), 5.21-5.17 (m, 1H), 2.19 (s, 4H), 2.17 (s, 4H), 2.03 (s, 3H), 1.52 (s, 2H). ¹³C NMR (176 MHz, dmso) δ 170.28, 167.22, 165.79, 149.50, 148.74, 145.19, 139.73, 135.08, 130.24, 128.98, 128.19, 127.41, 126.55, 125.97, 125.02, 122.58, 120.62, 117.41, 112.97, 55.89, 22.67, 12.09, 9.59, 3.60.

### (R,Z)-3-((4-acrylamido-3,5-dimethyl-1H-pyrrol-2-yl)methylene)-2-oxo-N-(1-phenylethyl)indoline-5-carboxamide (9b, RAR-12-32)

To a round bottom flask that contained 8a (50 mg, 0.12 mmol) were added 0.01 mL (0.12 mmol) of acrylic acid, 58.8 mg (0.12 mmol) of HATU, cat. DMAP and 0.50 mL (0.87 mmol) of TEA. All materials were dissolved in 2 mL of DMF and sonicated to give a homogenous solution. The solution was allowed to stir at 56° C. for two hours. Once complete, quenched with sat. LiCl and then extracted with EtOAc (3×30 mL). The combined organic layers were then dried over MgSO₄ and the material was purified by preparatory TLC (50% Acetone/Hexanes) collecting the baseline product, which was washed off the silica gel with acetone. The solvent was removed to give the final product as an orange solid, 13.9 mg, 24%. Molecular Formula: C₂₇H₂₆N₄O₃ ESI-MS calc: 454.20 ESI-MS found: 455.1632 [M+1] HPLC: 6.147 ¹H NMR (700 MHz, DMSO-d6) δ 13.53 (s, 1H), 11.07 (s, 1H), 9.41 (s, 1H), 8.60 (d, J=8.1 Hz, 2H), 8.21 (d, J=9.5 Hz, 1H), 7.69 (d, J=8.3 Hz, 2H), 7.66 (s, 1H), 7.41 (d, J=7.8 Hz, 5H), 7.33 (t, J=7.5 Hz, 5H), 7.22 (t, J=7.4 Hz, 3H), 6.93 (dd, J=8.1, 3.6 Hz, 1H), 6.45 (dd, J=17.1, 10.3 Hz, 1H), 6.23-6.19 (m, 1H), 5.72 (dd, J=10.7, 1.9 Hz, 1H), 5.19 (t, J=7.3 Hz, 2H), 2.21 (s, 3H), 2.20 (s, 3H), 1.50 (d, J=7.1 Hz, 3H). 13C NMR (176 MHz, dmso) δ 180.81, 169.69, 169.00, 167.87, 165.86, 153.99, 145.07, 141.22, 140.27, 136.23, 131.78, 129.29, 128.18, 127.56, 126.86, 126.49, 126.09, 124.16, 120.41, 109.40, 108.64, 53.84, 22.28.

### (R,Z)-3-((3,5-dimethyl-4-(vinylsulfonamido)-1H-pyrrol-2-yl)methylene)-2-oxo-N-(1-phenylethyl)indoline-5-carboxamide (9c, RAR-12-34)

Step 1: To a dried flask were added 0.03 mL (0.12 mmol) of vinyl sulfonic acid, 2 drops of DMF and 2 mL of DCM. The solution was cooled to 0° C. and then 0.02 mL (0.14 mmol) of oxalyl chloride were added in one portion. The solution was warmed to rt and allowed to stir until complete (1 hour). Once complete, then solvent was removed, and the resultant clear oil was rinsed with DCM (3×15 mL) and dried under high pressure until ready for second step Step 2: To the flask holding the acid chloride were added 8a (50 mg, 0.12 mmol) dissolved in 3 mL of THF. To this murky orange solution were added 0.50 mL (7 equiv) of TEA, and the solution was allowed to stir at rt for 5 hours. Once complete, removed solvent under pressure and brought yellow residue back up in EtOAc. The organic layer was washed with sat. Na2CO3 and then brine (1×30 mL) respectively. Purified by prepatory TLC plate (40% Acetone/Hexanes), collecting secondary spot (Rf=0.2-0.3). The material was rinsed off silica with acetone and the solvent was removed to give a dark orange solid as the desired product. Result: orange solid, 16 mg, 27% Molecular Formula: C₂₆H₂₆N₄O₄S ESI-MS calc: 490.17 ESI-MS found: 491.1249 HPLC: 6.43 ¹H NMR (400 MHz, DMSO-d6) δ 13.50 (s, 1H), 11.38 (s, 1H), 11.09 (s, 1H), 8.98 (s, 1H), 8.64-8.57 (m, 2H), 8.36 (s, 1H), 8.20 (s, 1H), 7.84 (s, 1H), 7.77 (d, J=8.3 Hz, 1H), 7.70 (d, J=8.2 Hz, 1H), 7.63 (s, 1H), 7.41 (d, J=7.8 Hz, 5H), 7.33 (t, J=7.6 Hz, 5H), 7.21 (d, J=7.3 Hz, 2H), 6.98 (s, 1H), 6.93 (d, J=8.1 Hz, 1H), 6.84 (dd, J=16.5, 9.8 Hz, 1H), 5.93 (d, J=8.0 Hz, 1H), 5.19 (t, J=7.4 Hz, 2H), 2.29 (s, 4H), 2.26 (s, 3H), 1.50 (d, J=7.1 Hz, 6H). 13C NMR (176 MHz, dmso) δ 169.90, 165.56, 145.07, 141.33, 136.84, 133.74, 128.32, 128.19, 127.65, 126.53, 126.15, 125.20, 124.79, 124.46, 123.83, 119.49, 119.12, 109.21, 48.52, 45.44, 39.88, 39.76, 39.64, 39.52, 39.40, 39.28, 39.16, 22.33, 14.83, 11.29, 8.63.

### (R,Z)-3-((4-(2-chloroacetamido)-3,5-dimethyl-1H-pyrrol-2-yl)methylene)-2-oxo-N-(1-phenylethyl)indoline-5-carboxamide (9d, RAR-12-52)

To a dried round bottom flask were added 8a (60 mg, 0.15 mmol) dissolved in 3 mL of THF. The yellow solution was cooled to 0° C. and 0.01 mL (0.18 mmol) of chloroacetylchloride was added dropwise. The solution was allowed to stir at 0° C. for 30 mins. Once complete by TLC, quenched with water and extracted with EtOAc. The solvent was removed, and the crude material was purified by prep plate (50% Acetone/Hexanes) collecting the major product. The desired product was rinsed off of silica gel and then the solvent was removed to give a bright yellow solid. The yellow solid was washed with DCM and sonicated to give a red solid. The red solid was then pulped in water:acetone (30:1) to give the final product. Result: red solid, 25.1 mg, 35% Molecular Formula: C₂₆H₂₅ClN₄O₃ ESI-MS calc: 476.16 ESI-MS found: 477.0999 HPLC: 6.388 ¹H NMR (700 MHz, DMSO-d6) δ 11.08 (s, 1H), 9.53 (s, 1H), 8.60 (d, J=8.0 Hz, 1H), 8.22 (s, 1H), 7.69 (d, J=8.2 Hz, 1H), 7.65 (s, 1H), 7.41 (d, J=7.8 Hz, 2H), 7.33 (t, J=7.5 Hz, 2H), 7.22 (t, J=7.4 Hz, 1H), 6.93 (d, J=8.0 Hz, 1H), 5.19 (t, J=7.5 Hz, −1H), 2.20 (d, J=12.1 Hz, 7H), 2.08 (s, 3H), 1.50 (d, J=7.1 Hz, 3H). 13C NMR (176 MHz, dmso) δ 169.67, 168.51, 156.78, 145.12, 140.30, 131.64, 129.98, 128.13, 127.50, 126.89, 126.44, 126.11, 124.45, 122.62, 121.01, 117.42, 116.93, 112.94, 48.39, 42.71, 22.29, 11.67, 9.24.

### (Z)-3-((4-((E)-4-(dimethylamino)but-2-enamido)-3,5-dimethyl-1H-pyrrol-2-yl)methylene)-2-oxo-N—((R)-1-phenylethyl)indoline-5-carboxamide (9e, RAR-12-54)

To a round bottom flask were added 8a (50 mg, 0.12 mmol), 20.2 mg (0.12 mmol) of N,N-dimethylaminobutenoic acid and 36 mg (0.12 mmol) of DMTMM and 0.20 mL (0.24 mmol) of TEA. All materials were brought up in 2 mL of DMF, and the resulting solution was allowed to stir at rt for 12 h. Once complete, the reaction was quenched with Sat. NaCl, and extracting with EtOAc (3×50 mL). The organic layer was washed with sat. Na2CO3 and brine (1×50 mL) respectively and then dried over MgSO₄. The solvent was removed under pressure, evaporating the material onto silica gel. Purified by column chromatography 5-100% Acetone/Hexanes. Flushed the column with 7N NH3 in MeOH to yield the final product as a bright yellow oil. Result: yellow oil, 13 mg, 20% Molecular Formula: C₃₀H₃₃N₅O₃ ESI-MS calc: 511.26 ESI-MS found: 512.2110 [M+1], 534.1905 [M+Na] HPLC: 5.424 ¹H NMR (700 MHz, DMSO-d6) δ 13.56 (s, 1H), 11.06 (s, 2H), 9.29 (s, 2H), 8.59 (d, J=7.9 Hz, 2H), 8.21 (d, J=1.5 Hz, 2H), 7.68 (d, J=8.1 Hz, 2H), 7.65 (s, 2H), 7.41 (d, J=7.6 Hz, 4H), 7.33 (t, J=7.6 Hz, 4H), 7.22 (t, J=7.5 Hz, 2H), 6.93 (d, J=8.1 Hz, 2H), 6.71-6.64 (m, 1H), 6.27 (d, J=15.5 Hz, 1H), 5.21-5.17 (m, 1H), 3.05 (d, J=5.6 Hz, 2H), 2.21 (s, 6H), 2.19 (s, 3H), 2.18 (s, 3H), 1.50 (d, J=7.1 Hz, 3H). 13C NMR (176 MHz, dmso) δ 170.09, 166.24, 163.98, 145.47, 141.00, 140.66, 132.25, 128.68, 128.55, 127.91, 127.30, 126.87, 126.48, 126.08, 125.81, 125.74, 125.61, 124.86, 124.52, 122.19, 117.61, 112.99, 109.03, 62.41, 48.96, 25.82, 22.64, 14.44, 12.32, 9.77.

### (R,Z)-3-((4-(2-bromoacetamido)-3,5-dimethyl-1H-pyrrol-2-yl)methylene)-2-oxo-N-(1-phenylethyl)indoline-5-carboxamide (9f RAR-12-98)

To a round bottom flask were added 83.7 mg of 8a (0.21 mmol), 60.1 mg (0.41 mmol) of bromoacetic acid and 100 mg (0.25 mmol) of DMTMM. All materials were brought up in 2 mL of DMF, and the resulting solution was allowed to stir at rt for 1.5 h. Once complete, the reaction was quenched with brine and then extracted with EtOAc (3×20 mL). The combined organic layer was dried over Na2SO4 and then solvent was removed under pressure to give a dark orange solid. The solid was then dissolved in DCM, and the resultant solution was sonicated to give a dark red precipitate. The solid was filtered off, and rinsed with excess cold DCM to give the final product. Result: red solid, 42.9 mg, 39%. Molecular Formula: C₂₆H₂₅BrN₄O₃ ESI-MS calc: 520.11 ESI-MS found: 523.1164 HPLC: 6.397 1H NMR (700 MHz, DMSO-d6) δ 13.52 (s, 1H), 11.08 (s, 1H), 9.60 (s, 1H), 8.60 (d, J=7.9 Hz, 1H), 8.21 (d, J=1.6 Hz, 1H), 7.69 (dd, J=8.1, 1.7 Hz, 1H), 7.65 (s, 1H), 7.41 (d, J=7.7 Hz, 2H), 7.33 (t, J=7.6 Hz, 2H), 7.22 (t, J=7.3 Hz, 1H), 6.93 (d, J=8.1 Hz, 1H), 5.20 (p, J=7.2 Hz, 1H), 4.03 (s, 2H), 2.20 (d, J=10.4 Hz, 6H), 1.51 (d, J=7.0 Hz, 3H). 13C NMR (176 MHz, dmso) δ 169.68, 165.82, 165.54, 145.05, 140.30, 131.64, 128.15, 127.59, 126.78, 126.47, 126.07, 125.76, 125.33, 124.44, 124.13, 121.01, 117.35, 112.95, 108.62, 48.34, 29.33, 22.26, 11.61, 9.14.

### (R,Z)-3-((4-(2-cyanoacetamido)-3,5-dimethyl-1H-pyrrol-2-yl)methylene)-2-oxo-N-(1-phenylethyl)indoline-5-carboxamide (9g RAR-12-67)

To a round bottom flask were added 20.2 mg of 8a (0.05 mmol), 6.7 mg (0.075 mmol) of cyanoacetic acid and 24.6 mg (0.1 mmol) of DMTMM. All materials were brought up in 2 mL of DMF, and the resulting solution was allowed to stir at rt for 12 h. Once complete, the reaction was quenched with Sat. NaCl, and extracting with EtOAc (3×50 mL). The organic layer was washed with sat. Na2CO3 and brine (1×50 mL) respectively and then dried over MgSO₄. The solvent was removed under pressure, evaporating the material onto silica gel. Purified by column chromatography 5-100% Acetone/Hexanes. Flushed the column with 7N NH3 in MeOH to yield the final product as a bright orange solid. Result: orange solid, 10 mg, 43% Molecular Formula: C₂₇H₂₅N₅O₃ ESI-MS calc: 467.20 ESI-MS found: 468.1297 HPLC: 6.100. 1H NMR (700 MHz, DMSO-d6) δ 13.51 (s, 1H), 11.08 (s, 1H), 9.52 (s, 1H), 8.60 (d, J=8.2 Hz, 1H), 8.21 (d, J=1.7 Hz, 1H), 7.69 (dd, J=8.1, 1.7 Hz, 1H), 7.65 (s, 1H), 7.41 (d, J=7.7 Hz, 2H), 7.33 (t, J=7.6 Hz, 3H), 7.22 (t, J=7.3 Hz, 1H), 6.93 (d, J=8.1 Hz, 1H), 5.19 (p, J=7.2 Hz, 1H), 3.88 (s, 2H), 2.20 (dd, J=12.9, 10.8 Hz, 6H), 1.50 (d, J=7.1 Hz, H).

### (Z)-3-((4-(3-chloro-2-hydroxypropanamido)-3,5-dimethyl-1H-pyrrol-2-yl)methylene)-2-oxo-N—((R)-1-phenylethyl)indoline-5-carboxamide (9h)

To a round bottom flask were added 80 mg of 8a (0.20 mmol), 31.1 mg (0.26 mmol) of 3-chloro-2-hydroxypropionic acid and 81.8 mg (0.32 mmol) of DMTMM. All materials were brought up in 2 mL of DMF, and the resulting solution was allowed to stir at rt for 12 h. Once complete, the reaction was quenched with Sat. NaCl, and extracting with EtOAc (3×50 mL). The organic layer was washed with sat. Na2CO3 and brine (1×50 mL) respectively and then dried over MgSO4. The solvent was removed under pressure, evaporating the material onto silica gel. Purified by column chromatography 5-100% Acetone/Hexanes. Result: orange solid, 72.8 mg, 58% Molecular Formula: C₂₇H₂₇ClN₄O₄ ESI-MS calc: 506.17 ESI-MS found: 507.1809 [M+1], 540.2377 [M+H+MeOH] HPLC: 6.043 1H NMR (700 MHz, DMSO-d6) δ 13.58-13.42 (m, 1H), 11.07 (d, J=4.9 Hz, 1H), 8.60 (d, J=8.1 Hz, 1H), 8.23-8.19 (m, 1H), 7.95 (s, 1H), 7.69-7.67 (m, 1H), 7.64 (d, J=5.9 Hz, 1H), 7.41 (d, J=7.2 Hz, 2H), 7.33 (t, J=7.1 Hz, 3H), 7.22 (t, J=7.2 Hz, 1H), 6.93 (dt, J=8.2, 2.1 Hz, 1H), 5.19 (p, J=7.3 Hz, 1H), 3.96 (d, J=7.9 Hz, 1H), 3.74 (s, 1H), 2.21-2.18 (m, 4H), 2.16 (s, 2H), 1.50 (d, J=7.0 Hz, 3H). 13C NMR (176 MHz, dmso) δ 172.44, 166.31, 162.74, 145.53, 128.63, 127.95, 126.94, 126.54, 125.88, 124.64, 121.80, 117.71, 115.92, 109.07, 107.36, 55.41, 48.82, 22.73, 21.51, 9.74.

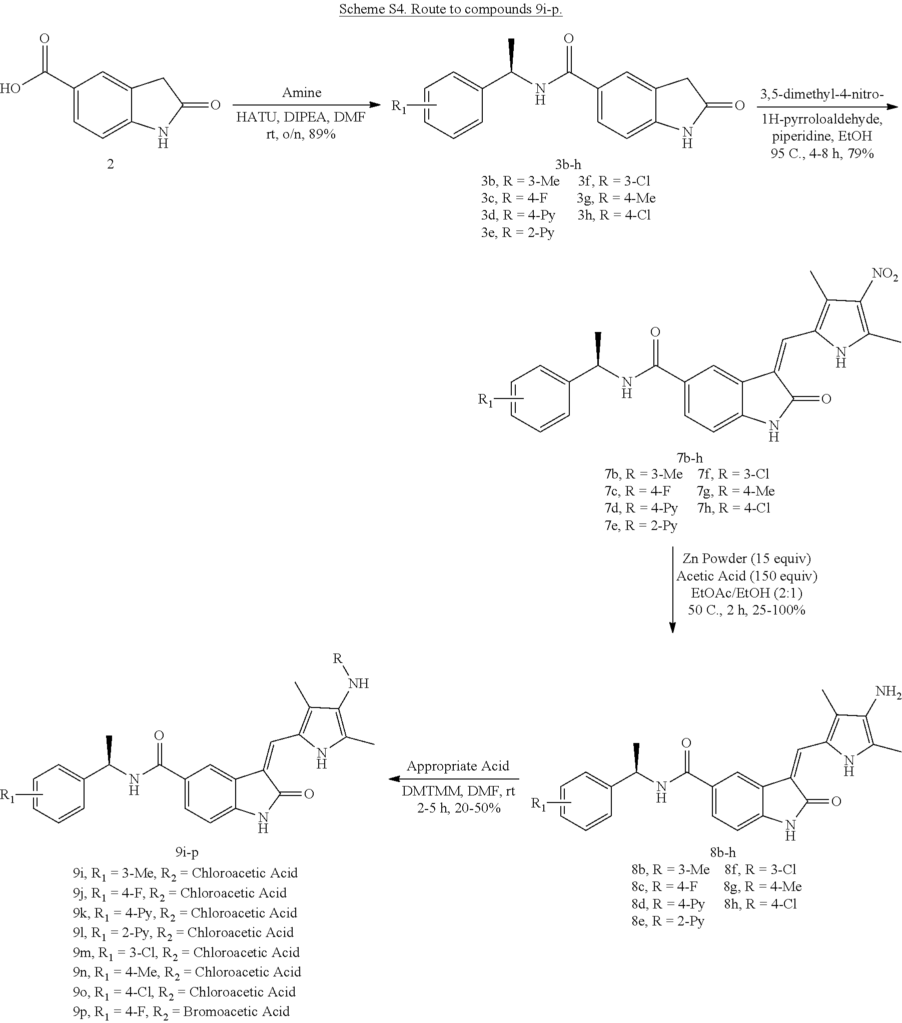

### (R)-2-oxo-N-(1-(m-tolyl)ethyl)indoline-5-carboxamide (3b RAR-12-63)

To a round bottom flask were added 236.1 mg (1.33 mmol) of 2-oxoindoline-5-carboxylic acid, dissolved in 7 mL of dry DMF. To this dark red solution were added 0.200 mL (1.47 mmol) of (R)-1-(m-tolyl)ethan-1-amine, 0.300 mL (1.73 mmol) of DIPEA and 510.2 mg (1.33 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 125.6 mg, 31% Molecular Formula: C₁₈H₁₈N₂O₂ ESI-MS calc: 294.14 ESI-MS found: 295.0167 [M+1] HPLC: 5.395 1H NMR (700 MHz, DMSO-d6) δ 10.61 (s, 1H), 8.58 (d, J=8.1 Hz, 1H), 7.77 (d, J=8.1 Hz, 2H), 7.18 (p, J=7.6 Hz, 4H), 7.02 (d, J=7.3 Hz, 1H), 6.85 (d, J=7.9 Hz, 1H), 5.11 (p, J=7.3 Hz, 1H), 2.28 (s, 3H), 1.44 (d, J=7.0 Hz, 3H). 13C NMR (176 MHz, dmso) δ 176.62, 165.16, 146.32, 145.07, 137.11, 128.06, 127.65, 127.48, 127.10, 126.66, 125.53, 123.51, 123.09, 108.36, 48.29, 39.86, 39.74, 39.62, 39.50, 39.38, 39.26, 39.14, 38.22, 35.57, 22.34, 21.11.

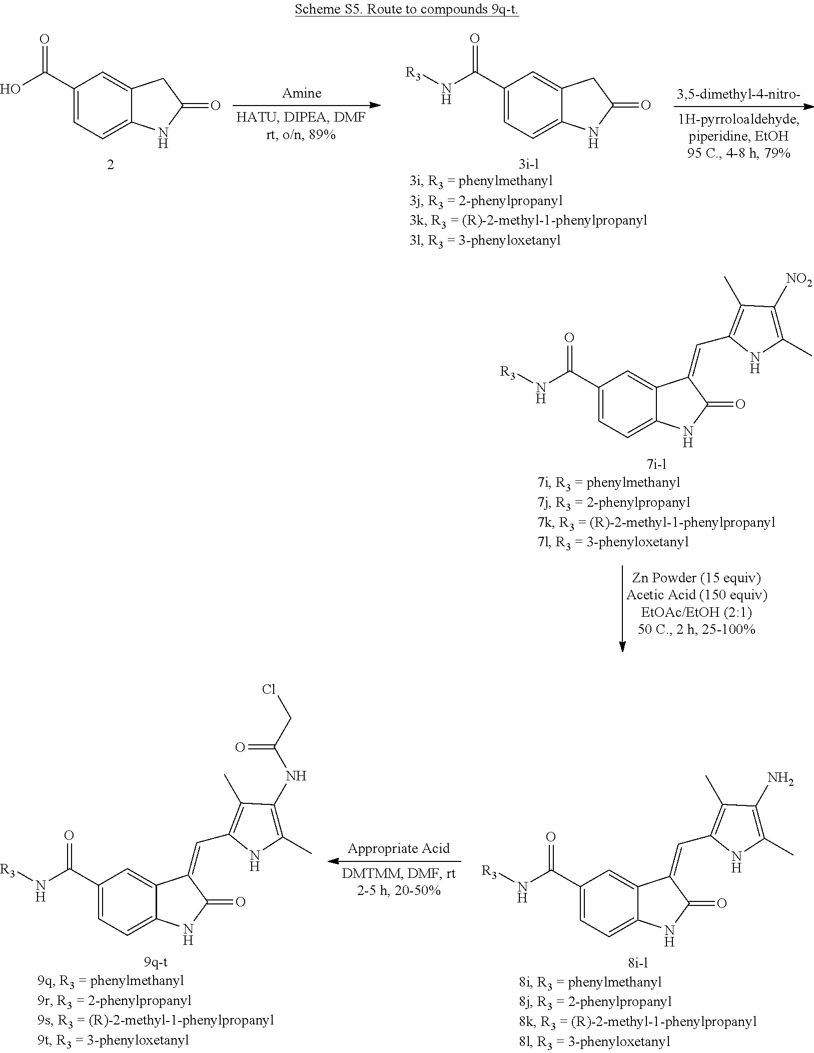

### (R)—N-(1-(4-fluorophenyl)ethyl)-2-oxoindoline-5-carboxamide (3c RAR-12-74)

To a round bottom flask were added 246.6 mg (1.41 mmol) of 2-oxoindoline-5-carboxylic acid, dissolved in 7 mL of dry DMF. To this dark red solution were added 0.200 mL (1.55 mmol) of (R)-1-(4-fluorophenyl)ethan-1-amine, 0.25 mL (1.41 mmol) of DIPEA and 667.1 mg (1.61 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 315 mg, 71% Molecular Formula: C₁₇H₁₅FN₂O₂ ESI-MS calc: 298.11 ESI-MS found: 299.1216 [M+1] HPLC: 5.414 1H NMR (500 MHz, DMSO-d6) δ 10.61 (s, 1H), 8.62 (d, J=8.0 Hz, 1H), 7.76 (d, J=7.5 Hz, 2H), 7.43-7.38 (m, 2H), 7.16-7.08 (m, 2H), 6.87-6.82 (m, 1H), 5.14 (p, J=7.2 Hz, 1H), 3.53 (s, 2H), 1.45 (d, J=7.1 Hz, 3H). 13C NMR (176 MHz, dmso) δ 176.84, 165.47, 160.34, 146.46, 141.34, 128.06, 128.01, 127.77, 127.49, 125.69, 123.63, 114.99, 114.87, 108.55, 47.90, 39.86, 39.74, 39.62, 39.50, 39.38, 39.26, 39.14, 38.33, 35.67, 22.35.

### (R)-2-oxo-N-(1-(pyridin-4-yl)ethyl)indoline-5-carboxamide (3d RAR-12-75)

To a round bottom flask were added 248.1 mg (1.41 mmol) of 2-oxoindoline-5-carboxylic acid, dissolved in 7 mL of dry DMF. To this dark red solution were added 0.200 mL (1.55 mmol) of (R)-1-(pyridin-4-yl)ethan-1-amine, 0.25 mL (1.41 mmol) of DIPEA and 652.4 mg (1.61 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 200 mg, 48% Molecular Formula: C₁₆H₁₅N₃O₂ ESI-MS calc: 281.12 ESI-MS found: 282.1236 [M+1] HPLC: 2.378 1H NMR (700 MHz, DMSO-d6) δ 10.64 (s, 1H), 8.74 (dd, J=7.6, 2.1 Hz, 1H), 8.55-8.52 (m, 2H), 7.79 (dd, J=7.5, 2.2 Hz, 2H), 7.45-7.42 (m, 2H), 7.08 (d, J=2.3 Hz, 1H), 7.01 (s, 1H), 6.89-6.85 (m, 1H), 5.14 (td, J=7.3, 2.2 Hz, 1H), 3.55 (s, 2H), 3.46-3.41 (m, 3H), 1.47 (dd, J=7.2, 2.3 Hz, 3H), 1.07-1.03 (m, 3H). 13C NMR (176 MHz, dmso) δ 176.63, 165.65, 155.16, 148.63, 146.56, 127.76, 127.03, 125.62, 123.57, 121.58, 108.43, 56.00, 47.92, 39.86, 39.74, 39.62, 39.50, 39.38, 39.26, 39.14, 35.56, 21.45, 18.54.

### (R)-2-oxo-N-(1-(pyridin-2-yl)ethyl)indoline-5-carboxamide (3e RAR-12-78)

To a round bottom flask were added 251.1 mg (1.41 mmol) of 2-oxoindoline-5-carboxylic acid, dissolved in 7 mL of dry DMF. To this dark red solution were added 0.200 mL (1.55 mmol) of (R)-1-(pyridin-2-yl)ethan-1-amine, 0.25 mL (1.41 mmol) of DIPEA and 625.1 mg (1.62 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 165 mg, 39% Molecular Formula: C₁₆H₁₅N₃O₂ ESI-MS calc: 281.12 ESI-MS found: 282.1236 [M+1] HPLC: 2.111 1H NMR (700 MHz, DMSO-d6) δ 10.62 (d, J=5.2 Hz, 1H), 8.63 (t, J=6.6 Hz, 1H), 8.51 (t, J=5.3 Hz, 1H), 7.80 (d, J=5.6 Hz, 2H), 7.74 (q, J=7.0 Hz, 1H), 7.38 (t, J=6.8 Hz, 1H), 7.24 (q, J=6.0 Hz, 1H), 6.86 (t, J=6.7 Hz, 1H), 5.17 (p, J=7.2 Hz, 1H), 3.54 (d, J=5.2 Hz, 3H), 1.49 (t, J=6.4 Hz, 3H). 13C NMR (176 MHz, dmso) δ 177.27, 166.16, 163.50, 149.12, 137.26, 128.18, 127.79, 126.11, 124.08, 122.46, 120.59, 108.98, 50.76, 40.22, 40.10, 39.98, 39.86, 39.74, 39.62, 39.50, 36.07, 21.45.

### (R)—N-(1-(3-chlorophenyl)ethyl)-2-oxoindoline-5-carboxamide (3f RAR-12-86)

To a round bottom flask were added 251.1 mg (1.41 mmol) of 2-oxoindoline-5-carboxylic acid, dissolved in 7 mL of dry DMF. To this dark red solution were added 0.200 mL (1.55 mmol) of (R)-1-(3-chlorophenyl)ethan-1-amine, 0.25 mL (1.41 mmol) of DIPEA and 717.7 mg (1.91 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 423.3 mg, 91% Molecular Formula: C₁₇H₁₅ClN₂O₂ ESI-MS calc: 314.08 ESI-MS found: 315.0438 [M+1] HPLC: 5.730 1H NMR (700 MHz, DMSO-d6) δ 10.64-10.57 (m, 1H), 8.65 (dd, J=7.5, 2.2 Hz, 1H), 7.78-7.74 (m, 2H), 7.41 (t, J=2.1 Hz, 1H), 7.33 (dt, J=6.7, 1.8 Hz, 2H), 7.26 (dp, J=6.4, 2.2 Hz, 1H), 6.85 (dd, J=8.1, 2.5 Hz, 1H), 5.15-5.06 (m, 1H), 3.53 (s, 2H), 1.45-1.42 (m, 3H). 13C NMR (176 MHz, dmso) δ 176.91, 165.61, 148.09, 146.73, 133.15, 130.39, 127.97, 126.74, 126.14, 125.88, 125.13, 123.78, 108.70, 56.27, 48.40, 40.12, 40.00, 39.88, 39.76, 39.64, 39.52, 39.40, 35.83, 22.43, 18.80.

### (R)-2-oxo-N-(1-(p-tolyl)ethyl)indoline-5-carboxamide (3g RAR-12-90)

To a round bottom flask were added 244.2 mg (1.41 mmol) of 2-oxoindoline-5-carboxylic acid, dissolved in 7 mL of dry DMF. To this dark red solution were added 0.220 mL (1.55 mmol) of (R)-1-(3-chlorophenyl)ethan-1-amine, 0.25 mL (1.41 mmol) of DIPEA and 622.1 mg (1.62 mmol) of HATU. The resultant dark red solution was allowed to stir at rt for 12 h, and then added 200 mL of sat. Na2CO3 and extracted with EtOAc (3×100 mL). The combined organic layer was washed with brine (2×50 mL) and then dried over MgSO₄. Purified by column chromatography (0-15% DCM/MeOH) to give the desired product as a strawberry pink solid. Yield: 300.8 mg, 69% Molecular Formula: C₁₈H₁₈N₂O₂ ESI-MS calc: 294.14 ESI-MS found: 317 [M+Na] HPLC: 5.556 1H NMR (700 MHz, DMSO-d6) δ 10.61 (s, 1H), 8.57 (d, J=8.1 Hz, 1H), 7.77 (d, J=8.1 Hz, 2H), 7.26 (d, J=7.8 Hz, 2H), 7.11 (d, J=7.8 Hz, 2H), 6.85 (d, J=8.0 Hz, 1H), 5.12 (p, J=7.3 Hz, 1H), 3.53 (s, 2H), 2.26 (s, 3H), 1.44 (d, J=7.0 Hz, 3H). 13C NMR (176 MHz, dmso) δ 176.63, 165.17, 146.30, 142.10, 135.44, 128.66, 127.64, 127.54, 125.93, 125.51, 123.51, 108.36, 48.03, 39.86, 39.74, 39.62, 39.50, 39.38, 39.26, 39.14, 35.57, 22.29, 20.59.

### (R)—N-(1-(4-chlorophenyl)ethyl)-2-oxoindoline-5-carboxamide (3h, RAR-13-16)

Prepared following the protocol described to construct 3. Yields a strawberry pink solid, 471.0 mg, quantitative yield. Molecular Formula: C17H15ClN₂O₂ ESI-MS calc: 314.08 ESI-MS found: 337.0717 [M+Na] HPLC: 5.682 1H NMR (500 MHz, DMSO-d6) δ 10.61 (s, 1H), 8.64 (d, J=7.9 Hz, 1H), 7.76 (d, J=7.9 Hz, 2H), 7.42-7.34 (m, 4H), 6.85 (d, J=8.1 Hz, 1H), 5.13 (p, J=7.2 Hz, 1H), 3.53 (s, 2H), 1.45 (d, J=7.0 Hz, 3H). 13C NMR (126 MHz, dmso) δ 176.61, 165.29, 146.40, 144.17, 130.98, 128.09, 127.92, 127.67, 127.28, 125.55, 123.49, 108.37, 47.87, 40.00, 39.83, 39.67, 39.50, 39.33, 39.17, 39.00, 35.55, 22.09.

### N-benzyl-2-oxoindoline-5-carboxamide (3i, RAR-12-100)

Prepared with protocol described in making 3. Yields a strawberry pink solid, 174.5 mg, 56% Molecular Formula: C₁₆H₁₄N₂O₂ ESI-MS calc: 266.11 ESI-MS found: 267.2180 [M+1] HPLC: 4.822 1H NMR (700 MHz, DMSO-d6) δ 10.61 (s, 1H), 8.87 (t, J=6.1 Hz, 1H), 7.79-7.75 (m, 2H), 7.34-7.28 (m, 5H), 7.23 (dt, J=7.2, 4.3 Hz, 2H), 6.86 (d, J=8.0 Hz, 1H), 4.46 (d, J=5.9 Hz, 2H), 3.53 (s, 2H). 13C NMR (176 MHz, dmso) δ 176.60, 165.95, 146.42, 139.88, 128.65, 128.19, 128.12, 127.70, 127.59, 127.47, 127.29, 127.11, 126.61, 125.66, 123.45, 108.44, 47.80, 45.00, 42.52, 39.86, 39.74, 39.62, 39.50, 39.38, 39.26, 39.14, 38.22, 35.57.

### 2-oxo-N-(2-phenylpropan-2-yl)indoline-5-carboxamide (3j, RAR-13-12)

Prepared using the protocol described for creation of 3. Yields a strawberry pink solid, 210.8 mg, 48%. Molecular Formula: C₁₈H₁₈N₂O₂ ESI-MS calc: 294.14 ESI-MS found: 295.1456 [M+1], 317.1275 [M+Na] HPLC: 5.199 1H NMR (700 MHz, DMSO-d6) δ 10.60 (s, 1H), 8.23 (s, 1H), 7.74 (s, 1H), 7.74-7.71 (m, 1H), 7.35 (d, J=7.8 Hz, 2H), 7.26 (t, J=7.7 Hz, 2H), 7.15 (t, J=7.2 Hz, 1H), 6.84 (d, J=8.1 Hz, 1H), 3.53 (s, 2H), 1.65 (s, 6H). 13C NMR (176 MHz, dmso) δ 176.64, 165.50, 148.19, 146.15, 128.51, 128.35, 127.82, 127.67, 125.59, 125.37, 124.81, 124.61, 123.65, 108.26, 55.22, 39.86, 39.74, 39.62, 39.50, 39.38, 39.26, 39.14, 35.59, 29.66.

### (R)—N-(2-methyl-1-phenylpropyl)-2-oxoindoline-5-carboxamide (3k, RAR-13-4)

Prepared using the protocol described for creation of 3. Yields a strawberry pink solid, 389.9 mg, quantitative yield. Molecular Formula: C₁₉H₂₀N₂O₂ ESI-MS calc: 308.15 ESI-MS found: 309.2172 HPLC: 5.793 1H NMR (700 MHz, DMSO-d6) δ 10.44 (s, 1H), 8.37 (d, J=8.9 Hz, 1H), 7.58 (dd, J=10.7, 2.9 Hz, 2H), 7.22 (d, J=7.6 Hz, 2H), 7.12 (t, J=7.5 Hz, 2H), 7.03 (t, J=7.3 Hz, 1H), 6.68 (d, J=8.0 Hz, 1H), 4.48 (t, J=9.2 Hz, 1H), 3.36 (s, 2H), 1.06 (d, J=6.4 Hz, 2H), 0.83 (d, J=6.5 Hz, 3H), 0.53 (d, J=6.7 Hz, 3H). 13C NMR (176 MHz, dmso) δ 176.69, 165.65, 164.62, 162.32, 146.32, 143.33, 128.02, 127.75, 127.70, 127.41, 126.61, 125.54, 123.53, 108.43, 59.92, 56.08, 41.67, 39.86, 39.74, 39.62, 39.50, 39.38, 39.26, 39.14, 38.23, 35.78, 35.62, 32.55, 30.77, 20.09, 19.90, 18.56.

### 2-oxo-N-(3-phenyloxetan-3-yl)indoline-5-carboxamide (31, RAR-13-8)

Prepared using the protocol described for the creation of 3. Yields a strawberry pink solid, 62.1 mg, 31% Molecular Formula: C₁₈H₁₆N₂O₃ ESI-MS calc: 308.12 ESI-MS found: 309.1235 HPLC: 4.601 1H NMR (700 MHz, DMSO-d6) δ 10.49 (d, J=4.6 Hz, 1H), 9.16 (d, J=4.6 Hz, 1H), 7.65-7.60 (m, 2H), 7.38-7.34 (m, 2H), 7.22-7.18 (m, 2H), 7.10 (tdd, J=7.3, 4.9, 2.2 Hz, 1H), 6.74-6.70 (m, 1H), 4.82 (dd, J=6.7, 4.8 Hz, 2H), 4.59 (dd, J=6.7, 4.8 Hz, 2H), 3.38 (d, J=4.7 Hz, 2H). 13C NMR (176 MHz, dmso) δ 176.66, 165.18, 146.78, 143.10, 129.97, 128.28, 128.17, 127.72, 126.91, 126.72, 126.06, 125.80, 125.41, 124.81, 123.53, 120.11, 108.72, 108.56, 81.89, 58.27, 53.49, 39.86, 39.74, 39.62, 39.50, 39.38, 39.26, 39.14, 38.23, 35.58, 35.52, 30.66, 14.07.

### (R,Z)-3-((3,5-dimethyl-4-nitro-1H-pyrrol-2-yl)methylene)-2-oxo-N-(1-(m-tolyl)ethyl)indoline-5-carboxamide (7b RAR-12-65)

To a dried sealed tube were added 200 mg (0.679 mmol) of (3b), 139.6 mg (0.815 mmol) of 3,5-dimethyl-4-nitro-1H-pyrrole-2-carbaldehyde, all of which were dissolved in abs. EtOH (6.0 mL). To this solution were added 0.05 ml of piperidine and heated to reflux (95° C.) for 4 h. Once complete, cooled to room temperature and then filtered off the product as a lemon yellow solid. Yield: lemon yellow solid, 100 mg, 29.8% Molecular Formula: C₂₅H₂₄N₄O₄ ESI-MS calc: 444.18 ESI-MS found: 445.1862 [M+1] HPLC: 7.732 1H NMR (700 MHz, DMSO-d6) δ 11.34 (s, 1H), 8.58 (d, J=8.0 Hz, 1H), 8.33 (d, J=1.6 Hz, 1H), 7.78 (s, 1H), 7.76 (dd, J=8.2, 1.6 Hz, 1H), 7.23-7.18 (m, 3H), 7.03 (d, J=6.1 Hz, 1H), 6.94 (d, J=8.1 Hz, 1H), 5.16 (p, J=7.2 Hz, 1H), 2.62 (s, 3H), 2.57 (s, 3H), 2.30 (s, 3H), 1.49 (d, J=7.0 Hz, 3H). 13C NMR (176 MHz, dmso) δ 169.83, 165.49, 144.90, 141.26, 137.14, 136.75, 133.66, 128.34, 128.09, 127.51, 127.15, 126.74, 125.07, 124.71, 124.39, 123.59, 123.17, 119.39, 118.96, 109.09, 48.36, 39.86, 39.74, 39.62, 39.50, 39.38, 39.26, 39.14, 22.26, 21.11, 14.76, 11.21.

### (R,Z)-3-((3,5-dimethyl-4-nitro-1H-pyrrol-2-yl)methylene)-N-(1-(4-fluorophenyl)ethyl)-2-oxoindoline-5-carboxamide (7c, RAR-12-76)

To a dried sealed tube were added 128 mg (0.429 mmol) of (3c), 101.3 mg (0.60 mmol) of 3,5-dimethyl-4-nitro-1H-pyrrole-2-carbaldehyde, all of which were dissolved in abs. EtOH (3.0 mL). To this solution were added 0.05 ml of piperidine and heated to reflux (95° C.) for 4 h. Once complete, cooled to room temperature and then filtered off the product as an orange solid. Yield: orange solid, 96.3 mg, 50% Molecular Formula: C₂₄H₂₁FN₄O₄ ESI-MS calc: 448.15 ESI-MS found: 449.1619 [M+1] HPLC: 7.422 1H NMR (700 MHz, DMSO-d6) δ 11.37 (s, 1H), 8.64 (d, J=7.8 Hz, 1H), 8.35 (d, J=1.7 Hz, 1H), 7.83 (s, 1H), 7.76 (dd, J=8.1, 1.7 Hz, 1H), 7.47-7.44 (m, 2H), 7.18-7.14 (m, 2H), 6.97 (d, J=8.1 Hz, 1H), 5.19 (p, J=7.2 Hz, 1H), 2.65 (s, 3H), 2.60 (s, 3H), 1.51 (d, J=7.0 Hz, 3H). 13C NMR (176 MHz, dmso) δ 169.85, 165.62, 136.79, 133.69, 128.31, 128.02, 127.97, 124.71, 123.71, 119.03, 114.88, 114.76, 50.22, 47.86, 39.86, 39.74, 39.62, 39.50, 39.38, 39.26, 39.14, 22.26, 14.78, 11.22.

### (R,Z)-3-((3,5-dimethyl-4-nitro-1H-pyrrol-2-yl)methylene)-2-oxo-N-(1-(pyridin-4-yl)ethyl)indoline-5-carboxamide (7d, RAR-12-77)

To a dried sealed tube were added 100 mg (0.455 mmol) of (3d), 77.1 mg (0.459 mmol) of 3,5-dimethyl-4-nitro-1H-pyrrole-2-carbaldehyde, all of which were dissolved in abs. EtOH (3.0 mL). To this solution were added 0.05 ml of piperidine and heated to reflux (95° C.) for 4 h. Once complete, cooled to room temperature and then filtered off the product as a yellow solid. Yield: yellow solid, 48.0 mg, 31.3% Molecular Formula: C₂₃H₂₁N₅O₄ ESI-MS calc: 431.16 ESI-MS found: 432.1656 [M+1] HPLC: 5.496 1H NMR (700 MHz, DMSO-d6) δ 11.36 (s, 1H), 8.73 (d, J=7.6 Hz, 1H), 8.52-8.50 (m, 2H), 8.36 (d, J=1.6 Hz, 1H), 7.82 (s, 1H), 7.77 (dd, J=8.1, 1.6 Hz, 1H), 7.40 (d, J=5.2 Hz, 2H), 6.97 (d, J=8.0 Hz, 1H), 5.15 (p, J=7.3 Hz, 1H), 2.64 (s, 3H), 2.59 (s, 3H), 1.51 (d, J=7.1 Hz, 3H). 13C NMR (176 MHz, dmso) δ 170.11, 166.24, 153.97, 149.80, 137.07, 135.63, 128.31, 127.80, 125.45, 124.98, 124.02, 121.55, 119.37, 109.42, 48.18, 40.12, 40.00, 39.88, 39.76, 39.64, 39.52, 39.40, 21.78, 15.04, 11.48.

### (R,Z)-3-((3,5-dimethyl-4-nitro-1H-pyrrol-2-yl)methylene)-2-oxo-N-(1-(pyridin-2-yl)ethyl)indoline-5-carboxamide (7e, RAR-12-83)

To a dried sealed tube were added 165 mg (0.587 mmol) of (3e), 122.8 mg (0.762 mmol) of 3,5-dimethyl-4-nitro-1H-pyrrole-2-carbaldehyde, all of which were dissolved in abs. EtOH (3.0 mL). To this solution were added 0.05 ml of piperidine and heated to reflux (95° C.) for 4 h. Once complete, cooled to room temperature and then filtered off the product as a yellow solid. Yield: yellow solid, 116 mg, 34% Molecular Formula: C₂₃H₂₁N₅O₄ ESI-MS calc: 431.16 ESI-MS found: 432.0558 [M+1] HPLC: 5.49 1H NMR (700 MHz, DMSO-d6) δ 11.36 (d, J=7.2 Hz, 1H), 8.65-8.58 (m, 1H), 8.55-8.47 (m, 1H), 8.39 (t, J=7.7 Hz, 1H), 7.78 (ddd, J=33.0, 15.8, 7.2 Hz, 3H), 7.42 (q, J=10.5, 9.0 Hz, 1H), 7.25 (dd, J=12.7, 6.8 Hz, 1H), 6.95 (d, J=7.0 Hz, 1H), 5.22 (q, J=8.8, 8.0 Hz, 1H), 2.66-2.60 (m, 3H), 2.59 (s, 3H), 1.56-1.49 (m, 3H). 13C NMR (176 MHz, dmso) δ 169.21, 165.90, 148.79, 136.83, 128.23, 124.86, 123.81, 120.44, 119.09, 109.32, 58.91, 50.44, 40.00, 39.88, 39.76, 39.74, 39.64, 39.62, 39.52, 39.40, 39.28, 21.09, 11.38.

### (R,Z)—N-(1-(3-chlorophenyl)ethyl)-3-((3,5-dimethyl-4-nitro-1H-pyrrol-2-yl)methylene)-2-oxoindoline-5-carboxamide (7f RAR-12-87)

To a dried sealed tube were added 242.2 mg (0.769 mmol) of (3f), 150.1 mg (0.892 mmol) of 3,5-dimethyl-4-nitro-1H-pyrrole-2-carbaldehyde, all of which were dissolved in abs. EtOH (6.0 mL). To this solution were added 0.05 ml of piperidine and heated to reflux (95° C.) for 4 h. Once complete, cooled to room temperature and then filtered off the product as an orange solid. Yield: orange solid, 46 mg, 16% Molecular Formula: C₂₄H₂₁ClN₄O₄ ESI-MS calc: 464.13 ESI-MS found: 465.1318 HPLC: 7.699 1H NMR (700 MHz, DMSO-d6) δ 11.38 (s, 1H), 8.68 (d, J=7.8 Hz, 1H), 8.36 (d, J=1.6 Hz, 1H), 7.84 (s, 1H), 7.76 (dd, J=8.2, 1.7 Hz, 1H), 7.46 (d, J=2.1 Hz, 1H), 7.37 (d, J=6.6 Hz, 2H), 7.29 (dt, J=6.7, 2.3 Hz, 1H), 6.97 (d, J=8.1 Hz, 1H), 5.17 (p, J=7.2 Hz, 1H), 2.65 (s, 3H), 2.60 (s, 3H), 1.50 (d, J=7.1 Hz, 3H). 13C NMR (176 MHz, dmso) δ 165.88, 147.85, 144.31, 138.20, 130.27, 126.64, 126.10, 125.34, 125.04, 123.98, 119.28, 109.31, 102.54, 48.41, 40.00, 39.88, 39.76, 39.64, 39.52, 39.40, 39.28, 22.27, 14.93.

### (R,Z)-3-((3,5-dimethyl-4-nitro-1H-pyrrol-2-yl)methylene)-2-oxo-N-(1-(p-tolyl)ethyl)indoline-5-carboxamide (7g, RAR-12-91)

To a dried sealed tube were added 253.9 mg (0.862 mmol) of (3g), 140.9 mg (0.837 mmol) of 3,5-dimethyl-4-nitro-1H-pyrrole-2-carbaldehyde, all of which were dissolved in abs. EtOH (3.0 mL). To this solution were added 0.05 ml of piperidine and heated to reflux (95° C.) for 4 h. Once complete, cooled to room temperature and then filtered off the product as an orange solid. Yield: orange solid, 76.1 mg, 25% Molecular Formula: C₂₅H₂₄N₄O₄ ESI-MS calc: 444.18 ESI-MS found: 445.1864 [M+1] HPLC: 7.662 1H NMR (700 MHz, DMSO-d6) δ 9.73 (s, 1H), 8.57 (d, J=8.2 Hz, 2H), 8.35 (d, J=1.7 Hz, 1H), 7.83 (s, 1H), 7.76 (dd, J=8.1, 1.7 Hz, 2H), 7.30 (d, J=7.8 Hz, 3H), 7.14 (d, J=7.9 Hz, 3H), 6.96 (d, J=8.0 Hz, 2H), 5.17 (p, J=7.2 Hz, 1H), 2.55 (d, J=8.7 Hz, 6H), 2.28 (s, 5H). 13C NMR (176 MHz, dmso) δ 170.11, 165.77, 155.70, 142.17, 141.51, 137.04, 135.76, 135.76, 128.94, 126.27, 125.36, 119.23, 109.39, 52.91, 40.12, 40.00, 39.88, 39.76, 39.64, 39.52, 39.40, 22.49, 20.86, 15.04, 11.49, 10.19.

### (Z)—N-benzyl-3-((3,5-dimethyl-4-nitro-1H-pyrrol-2-yl)methylene)-2-oxoindoline-5-carboxamide (7i, R AR-13-1)

Prepared using the protocol described for the creation of 7. Yields an orange solid, 150 mg, 55% Molecular Formula: C₂₃H₂₀N₄O₄ ESI-MS calc: 416.15 ESI-MS found: 417.2934, HPLC: 7.158, 1H NMR (700 MHz, DMSO-d6) δ 11.44-11.06 (m, 1H), 8.84 (t, J=6.1 Hz, 1H), 8.34 (s, 1H), 7.76 (d, J=8.3 Hz, 1H), 7.69 (s, 1H), 7.34 (d, J=5.6 Hz, 4H), 7.25 (d, J=6.6 Hz, 1H), 6.92 (d, J=8.2 Hz, 1H), 4.52 (d, J=5.7 Hz, 2H). 13C NMR (176 MHz, dmso) δ 169.75, 166.10, 141.33, 139.82, 136.69, 133.57, 128.23, 127.89, 127.37, 127.18, 126.67, 124.95, 124.69, 124.44, 123.25, 119.29, 118.57, 109.16, 42.62, 39.86, 39.74, 39.62, 39.50, 39.38, 39.26, 39.14, 14.73, 11.11.

### (Z)-3-((3,5-dimethyl-4-nitro-1H-pyrrol-2-yl)methylene)-2-oxo-N-(2-phenylpropan-2-yl)indoline-5-carboxamide (7j, RAR-13-13)

Yields an orange solid, 76.1 mg, 32.1%. Molecular Formula: C₂₅H₂₄N₄O₄ ESI-MS calc: 444.18 ESI-MS found:445.2680 [M+1] HPLC: 7.713 1H NMR (700 MHz, DMSO-d6) δ 11.32 (s, 1H), 8.31 (d, J=1.7 Hz, 1H), 8.25 (s, 1H), 7.80 (s, 1H), 7.72 (dd, J=8.0, 1.7 Hz, 1H), 7.42-7.39 (m, 2H), 7.28 (t, J=7.8 Hz, 2H), 7.17 (t, J=7.3 Hz, 1H), 6.92 (d, J=8.1 Hz, 1H), 2.61 (s, 3H), 2.57 (s, 3H), 1.70 (s, 6H). 13C NMR (176 MHz, dmso) δ 169.84, 165.70, 148.16, 141.11, 136.71, 133.63, 129.19, 127.83, 127.58, 125.62, 125.05, 124.73, 124.69, 124.29, 123.61, 119.44, 119.02, 108.96, 55.31, 39.86, 39.74, 39.62, 39.50, 39.38, 39.26, 39.14, 29.65, 14.76, 11.21.

### (R,Z)-3-((3,5-dimethyl-4-nitro-1H-pyrrol-2-yl)methylene)-N-(2-methyl-1-phenylpropyl)-2-oxoindoline-5-carboxamide (7k, RAR-13-5)

Prepared using the protocol described for creation of 7. Yields an orange solid, 144.6 mg, 64.8% Molecular Formula: C₂₆H₂₆N₄O₄ ESI-MS calc: 458.20 ESI-MS found: 459.2109 HPLC: 7.896 1H NMR (700 MHz, DMSO-d6) δ 11.32 (s, 1H), 8.56 (p, J=9.3, 8.3 Hz, 1H), 8.29 (dq, J=13.4, 7.5 Hz, 1H), 7.88-7.63 (m, 2H), 7.48-7.28 (m, 4H), 7.22 (qd, J=14.5, 9.4, 7.5 Hz, 1H), 6.94 (tt, J=14.2, 7.5 Hz, 1H), 4.79-4.64 (m, 1H), 2.64-2.58 (m, 3H), 2.58 (s, 3H), 1.05 (tt, J=13.4, 8.0 Hz, 3H), 0.75 (dp, J=25.8, 6.7 Hz, 3H). 13C NMR (176 MHz, dmso) δ 169.69, 165.86, 143.12, 141.05, 127.89, 127.29, 127.17, 126.47, 125.00, 124.61, 124.32, 123.62, 119.24, 119.05, 108.85, 59.80, 39.74, 39.62, 39.50, 39.38, 39.26, 39.15, 39.03, 32.40, 19.97, 19.82, 14.64, 11.05.

### (Z)-3-((3,5-dimethyl-4-nitro-1H-pyrrol-2-yl)methylene)-2-oxo-N-(3-phenyloxetan-3-yl)indoline-5-carboxamide (71, RAR-13-9)

Prepared using the protocol described for the creation of 7. Yields an orange solid, 47 mg, 42%. Molecular Formula: C₂₅H₂₂N₄O₅ ESI-MS calc: 458.16 ESI-MS found: 459.1656 HPLC: 6.923, 1H NMR (700 MHz, DMSO-d6) δ 11.39 (s, 1H), 9.36 (s, 1H), 8.40 (d, J=2.0 Hz, 1H), 7.83 (d, J=2.2 Hz, 1H), 7.81-7.77 (m, 1H), 7.59-7.55 (m, 2H), 7.40 (t, J=7.8 Hz, 2H), 7.29 (t, J=7.4 Hz, 1H), 6.99 (dd, J=8.4, 2.0 Hz, 1H), 5.04 (d, J=6.7 Hz, 2H), 4.81 (d, J=6.7 Hz, 2H), 2.63 (d, J=2.0 Hz, 3H), 2.58 (d, J=2.1 Hz, 3H). 13C NMR (176 MHz, dmso) δ 169.85, 165.50, 142.99, 141.58, 136.84, 133.69, 127.65, 127.44, 126.92, 125.26, 124.89, 124.72, 124.58, 123.78, 119.24, 109.26, 81.88, 58.33, 14.77, 11.15.

### (R,Z)—N-(1-(4-chlorophenyl)ethyl)-3-((3,5-dimethyl-4-nitro-1H-pyrrol-2-yl)methylene)-2-oxoindoline-5-carboxamide (7k, RAR-13-17)

Prepared using the protocol described for the creation of 7. Yields an orange solid, 213.3 mg, 96%. Molecular Formula: C₂₄H₂₁ClN₄O₄ ESI-MS calc: 464.13 ESI-MS found: 465.1318 [M+1] HPLC: 7.795 1H NMR (500 MHz, DMSO-d6) δ 11.34 (s, 1H), 8.65 (d, J=7.8 Hz, 1H), 8.35-8.30 (m, 1H), 7.79 (s, 1H), 7.75 (dd, J=8.1, 1.7 Hz, 1H), 7.43 (d, J=8.3 Hz, 2H), 7.39 (d, J=8.5 Hz, 3H), 6.94 (d, J=8.1 Hz, 1H), 5.17 (p, J=7.2 Hz, 1H), 2.62 (s, 3H), 2.57 (s, 3H), 1.49 (d, J=7.1 Hz, 3H). 13C NMR (126 MHz, dmso) δ 169.83, 165.67, 144.05, 141.32, 136.76, 133.66, 131.02, 128.18, 128.10, 128.00, 127.49, 125.11, 124.70, 124.41, 123.63, 119.35, 118.98, 109.10, 48.00, 40.00, 39.83, 39.67, 39.50, 39.34, 39.17, 39.00, 22.07, 14.76, 11.21.

### (R,Z)-3-((4-amino-3,5-dimethyl-1H-pyrrol-2-yl)methylene)-2-oxo-N-(1-(m-tolyl)ethyl)indoline-5-carboxamide (8b, RAR-12-68)

To a flask were added 86.4 mg of (7b), dissolved in 5 mL of 2:1 EtOH/EtOAc. To this slurry were added 210.9 mg (14 equiv) of Zn powder and 2 mL (150 equiv) of AcOH. The turbid orange solution was allowed to stir at 50 C for 2 h. Once complete, the reaction was cooled to rt and then add EtOAc before basifying with sat. Na2CO3. The basified aqueous layer was extracted with EtOAc (3×30 mL) and then washed with water and brine (1×30 mL), respectively. The organic layer was dried over MgSO4 and then solvent was removed under pressure to give the desired product as a red solid. *Note: the free amine is very reactive, so it was moved forward without further characterization* Result: red solid, 86.6 mg, 94% Molecular Formula: C₂₅H₂₆N₄O₂ ESI-MS calc: 414.21 ESI-MS found: 415.2120 [M+1] HPLC: 5.627

### (R,Z)-3-((4-amino-3,5-dimethyl-1H-pyrrol-2-yl)methylene)-N-(1-(4-fluorophenyl)ethyl)-2-oxoindoline-5-carboxamide (8c, RAR-12-79)

To a flask were added 96.3 mg of (7c), dissolved in 5 mL of 2:1 EtOH/EtOAc. To this slurry were added 295 mg (14 equiv) of Zn powder and 2 mL (150 equiv) of AcOH. The turbid orange solution was allowed to stir at 50 C for 2 h. Once complete, the reaction was cooled to rt and then add EtOAc before basifying with sat. Na2CO3. The basified aqueous layer was extracted with EtOAc (3×30 mL) and then washed with water and brine (1×30 mL), respectively. The organic layer was dried over MgSO4 and then solvent was removed under pressure to give the desired product as a red solid. *Note: the free amine is very reactive, so it was moved forward without further characterization* Result: red solid, 96 mg, 100% Molecular Formula: C₂₄H₂₃FN₄O₂ ESI-MS calc: 418.18 ESI-MS found: 419.1938 [M+1] HPLC: 5.713

### (R,Z)-3-((4-amino-3,5-dimethyl-1H-pyrrol-2-yl)methylene)-2-oxo-N-(1-(pyridin-4-yl)ethyl)indoline-5-carboxamide (8d RAR-12-80)

To a flask were added 48.0 mg of (7d), dissolved in 5 mL of 2:1 EtOH/EtOAc. To this slurry were added 208 mg (14 equiv) of Zn powder and 2 mL (150 equiv) of AcOH. The turbid orange solution was allowed to stir at 50 C for 2 h. Once complete, the reaction was cooled to rt and then add EtOAc before basifying with sat. Na2CO3. The basified aqueous layer was extracted with EtOAc (3×30 mL) and then washed with water and brine (1×30 mL), respectively. The organic layer was dried over MgSO4 and then solvent was removed under pressure to give the desired product as a red solid. *Note: the free amine is very reactive, so it was moved forward without further characterization* Result: red solid, 41.5 mg, 89% Molecular Formula: C₂₃H₂₃N₅O₂ ESI-MS calc: 401.19 ESI-MS found: 402.1920 [M+1] HPLC: 3.928

### (R,Z)-3-((4-amino-3,5-dimethyl-1H-pyrrol-2-yl)methylene)-2-oxo-N-(1-(pyridin-2-yl)ethyl)indoline-5-carboxamide (8e, RAR-12-84)

To a flask were added 73.9 mg of (7e), dissolved in 5 mL of 2:1 EtOH/EtOAc. To this slurry were added 289 mg (14 equiv) of Zn powder and 2 mL (150 equiv) of AcOH. The turbid orange solution was allowed to stir at 50 C for 2 h. Once complete, the reaction was cooled to rt and then add EtOAc before basifying with sat. Na2CO3. The basified aqueous layer was extracted with EtOAc (3×30 mL) and then washed with water and brine (1×30 mL), respectively. The organic layer was dried over MgSO4 and then solvent was removed under pressure to give the desired product as a red solid. *Note: the free amine is very reactive, so it was moved forward without further characterization* Result: red solid, 73.8 mg, 100% Molecular Formula: C₂₃H₂₃N₅O₂ ESI-MS calc: 401.19 ESI-MS found: 402.1450 [M+1] HPLC: 3.920

### (R,Z)-3-((4-amino-3,5-dimethyl-1H-pyrrol-2-yl)methylene)-N-(1-(3-chlorophenyl)ethyl)-2-oxoindoline-5-carboxamide (8f RAR-12-88)

To a flask were added 46.0 mg of (7f), dissolved in 5 mL of 2:1 EtOH/EtOAc. To this slurry were added 170.1 mg (14 equiv) of Zn powder and 1.4 mL (150 equiv) of AcOH. The turbid orange solution was allowed to stir at 50 C for 2 h. Once complete, the reaction was cooled to rt and then add EtOAc before basifying with sat. Na2CO3. The basified aqueous layer was extracted with EtOAc (3×30 mL) and then washed with water and brine (1×30 mL), respectively. The organic layer was dried over MgSO₄ and then solvent was removed under pressure to give the desired product as a red solid. *Note: the free amine is very reactive, so it was moved forward without further characterization* Result: red solid, 40 mg, 95% Molecular Formula: C₂₄H₂₃ClN₄O₂ ESI-MS calc: 434.15 ESI-MS found: 435.1569 [M+1] HPLC: 5.556

### (R,Z)-3-((4-amino-3,5-dimethyl-1H-pyrrol-2-yl)methylene)-2-oxo-N-(1-(p-tolyl)ethyl)indoline-5-carboxamide (8g RAR-12-92)

To a flask were added 76.1 mg of (7g), dissolved in 5 mL of 2:1 EtOH/EtOAc. To this slurry were added 314.4 mg (14 equiv) of Zn powder and 2.0 mL (150 equiv) of AcOH. The turbid orange solution was allowed to stir at 50 C for 2 h. Once complete, the reaction was cooled to rt and then add EtOAc before basifying with sat. Na2CO3. The basified aqueous layer was extracted with EtOAc (3×30 mL) and then washed with water and brine (1×30 mL), respectively. The organic layer was dried over MgSO₄ and then solvent was removed under pressure to give the desired product as a red solid. *Note: the free amine is very reactive, so it was moved forward without further characterization* Result: red solid, 220 mg, 100% Molecular Formula: C₂₅H₂₆N₄O₂ ESI-MS calc: 414.21 ESI-MS found: 415.2119 [M+1] HPLC: 5.573

### (R,Z)-3-((4-amino-3,5-dimethyl-1H-pyrrol-2-yl)methylene)-N-(1-(4-chlorophenyl)ethyl)-2-oxoindoline-5-carboxamide (8h, RAR-13-18)

Synthesized using protocol described n 8a. Yields a red solid, 36.1 mg, 48%. Molecular Formula: C24H23ClN₄O₂ ESI-MS calc: 434.15 ESI-MS found: 435.15612 HPLC: 5.398

### (Z)-3-((4-amino-3,5-dimethyl-1H-pyrrol-2-yl)methylene)-N-benzyl-2-oxoindoline-5-carboxamide (8i, RAR-13-2)

Synthesized using protocol described in 8a. Yields a red solid, 210 mg, quantitative yield. Molecular Formula: C₂₃H₂₂N₄O₂ ESI-MS calc: 386.17 ESI-MS found: 387.1810 HPLC: 5.129.

### (Z)-3-((4-amino-3,5-dimethyl-1H-pyrrol-2-yl)methylene)-2-oxo-N-(2-phenylpropan-2-yl)indoline-5-carboxamide (8j, RAR-13-14)

Synthesized using protocol described in 8a. Yields a red solid, 307 mg, quantitative yield. Molecular Formula: C₂₅H₂₆N₄O₂ ESI-MS calc: 414.21 ESI-MS found: 415.2119 [M+1] HPLC: 5.245

### (R,Z)-3-((4-amino-3,5-dimethyl-1H-pyrrol-2-yl)methylene)-N-(2-methyl-1-phenylpropyl)-2-oxoindoline-5-carboxamide (8k, RAR-13-6)

Synthesized using protocol described in 8a. Yields a red solid, 220 mg, quantitative yield. Molecular Formula: C₂₆H₂₈N₄O₂ ESI-MS calc: 428.22 ESI-MS found: 429.2201 HPLC: 5.497

### (Z)-3-((4-amino-3,5-dimethyl-1H-pyrrol-2-yl)methylene)-2-oxo-N-(3-phenyloxetan-3-yl)indoline-5-carboxamide (81, RAR-13-10)

Synthesized using protocol described in 8a. Yields a red solid, 20.6 mg, 47%. Molecular Formula: C₂₅H₂₄N₄O₃ ESI-MS calc: 428.18 ESI-MS found: 429.1494 HPLC: 4.710

### (R,Z)-3-((4-(2-chloroacetamido)-3,5-dimethyl-1H-pyrrol-2-yl)methylene)-2-oxo-N-(1-(m-tolyl)ethyl)indoline-5-carboxamide (9i RAR-12-69)

To a dried round bottom flask were added 86.6 mg of 8b, (0.209 mmol) dissolved in 3 mL of THF. The yellow solution was cooled to 0° C. and 0.04 mL (0.30 mmol) of chloroacetylchloride was added dropwise. The solution was allowed to stir at 0° C. for 30 mins. Once complete by TLC, quenched with water and extracted with EtOAc. The solvent was removed, and the crude material was purified by prep plate (50% Acetone/Hexanes) collecting the major product. The desired product was rinsed off of silica gel and then the solvent was removed to give a bright yellow solid. The yellow solid was washed with DCM and sonicated to give a red solid. The red solid was then pulped in water:acetone (30:1) to give the final product. Result: red solid, 57.2 mg, 55% Molecular Formula: C₂₇H₂₇ClN₄O₃ ESI-MS calc: 490.18 ESI-MS found: 491.1133 HPLC: 6.68 1H NMR (500 MHz, DMSO-d6) δ 13.52 (s, 1H), 11.07 (s, 1H), 9.53 (s, 1H), 8.56 (d, J=8.1 Hz, 1H), 8.21 (s, 1H), 7.74-7.62 (m, 2H), 7.24-7.15 (m, 4H), 7.03 (s, 1H), 6.93 (d, J=8.0 Hz, 1H), 5.15 (s, 1H), 4.26 (s, 2H), 2.30 (s, 3H), 2.20 (d, J=8.9 Hz, 6H), 1.49 (d, J=7.0 Hz, 3H). 13C NMR (126 MHz, dmso) δ 170.18, 169.06, 166.24, 165.84, 145.51, 140.80, 137.63, 132.18, 128.59, 128.10, 127.62, 127.24, 126.26, 125.83, 124.93, 123.68, 121.47, 117.93, 113.45, 109.13, 48.80, 43.22, 22.79, 21.62, 12.15, 9.70.

### (R,Z)-3-((4-(2-chloroacetamido)-3,5-dimethyl-1H-pyrrol-2-yl)methylene)-N-(1-(4-fluorophenyl)ethyl)-2-oxoindoline-5-carboxamide (9j, RAR-12-81)

To a dried round bottom flask were added 96.0 mg of 8c, (0.23 mmol) dissolved in 4 mL of THF. The yellow solution was cooled to 0° C. and 0.02 mL (0.25 mmol) of chloroacetylchloride was added dropwise. The solution was allowed to stir at 0° C. for 30 mins. Once complete by TLC, quenched with water and extracted with EtOAc. The solvent was removed, and the crude material was purified by prep plate (50% Acetone/Hexanes) collecting the major product. The desired product was rinsed off of silica gel and then the solvent was removed to give a bright yellow solid. The yellow solid was washed with DCM and sonicated to give a red solid. The red solid was then pulped in water:acetone (30:1) to give the final product. Result: red solid, 46.5 mg, 41% Molecular Formula: C₂₆H₂₄FClN₄O₃ ESI-MS calc: 494.15 ESI-MS found: 495.0480 [M+1] HPLC: 6.439 1H NMR (700 MHz, DMSO-d6) δ 13.52 (s, 1H), 11.08 (s, 1H), 9.54 (s, 1H), 8.63 (d, J=7.9 Hz, 1H), 8.23 (d, J=1.7 Hz, 1H), 7.68 (dd, J=8.1, 1.8 Hz, 1H), 7.66 (s, 1H), 7.46-7.43 (m, 2H), 7.15 (tt, J=9.9, 3.2 Hz, 3H), 6.93 (d, J=8.2 Hz, 1H), 5.19 (p, J=7.2 Hz, 1H), 4.28-4.26 (m, 4H), 2.20 (d, J=10.1 Hz, 6H), 1.50 (d, J=6.9 Hz, 4H). 13C NMR (176 MHz, dmso) δ 170.20, 169.07, 166.36, 165.86, 162.12, 160.75, 141.78, 141.76, 140.85, 132.22, 128.54, 128.49, 128.41, 128.02, 127.42, 126.27, 125.87, 124.96, 124.70, 121.50, 117.90, 115.38, 115.26, 113.44, 109.15, 48.33, 43.23, 41.97, 40.37, 40.25, 40.13, 40.01, 39.89, 39.77, 39.65, 36.07, 29.51, 22.78, 12.16, 9.72.

### (R,Z)-3-((4-(2-chloroacetamido)-3,5-dimethyl-1H-pyrrol-2-yl)methylene)-2-oxo-N-(1-(pyridin-4-yl)ethyl)indoline-5-carboxamide (9k, RAR-12-82)

To a dried round bottom flask were added 41.5 mg of 8d, (0.103 mmol) dissolved in 2 mL of THF. The yellow solution was cooled to 0° C. and 0.01 mL (0.103 mmol) of chloroacetylchloride was added dropwise. The solution was allowed to stir at 0° C. for 30 mins. Once complete by TLC, quenched with water and extracted with EtOAc. The solvent was removed, and the crude material was purified by prep plate (50% Acetone/Hexanes) collecting the major product as the desired product. Result: red solid, 6.3 mg, 13% Molecular Formula: C₂₅H₂₄ClN₅O₃ ESI-MS calc: 477.16 ESI-MS found: 478.1734 [M+H] HPLC: 4.363 1H NMR (700 MHz, DMSO-d6) δ 13.51 (s, 1H), 11.15 (s, 1H), 10.56 (s, 1H), 9.86 (s, 1H), 8.73 (s, 1H), 8.48 (s, 2H), 7.75 (s, 1H), 7.72 (d, J=7.9 Hz, 1H), 7.34 (d, J=7.6 Hz, 2H), 7.27 (s, 1H), 6.96 (d, J=7.9 Hz, 1H), 5.33 (t, J=7.0 Hz, 1H), 4.27 (s, 2H), 2.21 (s, 6H), 1.50 (d, J=7.8 Hz, 3H). 13C NMR (176 MHz, dmso) δ 168.53, 166.49, 165.34, 154.01, 141.60, 140.65, 130.38, 129.30, 129.05, 127.97, 124.37, 123.04, 122.68, 121.09, 119.53, 118.36, 108.73, 54.90, 42.72, 21.66, 10.38, 8.77.

### (R,Z)-3-((4-(2-chloroacetamido)-3,5-dimethyl-1H-pyrrol-2-yl)methylene)-2-oxo-N-(1-(pyridin-2-yl)ethyl)indoline-5-carboxamide (9l, RAR-12-85)

To a dried round bottom flask were added 73.8 mg of 8e, (0.18 mmol) dissolved in 4 mL of THF. The yellow solution was cooled to 0° C. and 0.04 mL (0.50 mmol) of chloroacetylchloride was added dropwise. The solution was allowed to stir at 0° C. for 30 mins. Once complete by TLC, removed solvent under pressure and then added DCM to the dark red residue. The DCM solution was sonicated to give a dark red precipitate. The solid was filtered off and rinsed with THF and DCM to give the desired product as a dark red solid. Result: red solid, 37.1 mg, 42% Molecular Formula: C₂₅H₂₄ClN₅O₃ ESI-MS calc: 477.16 ESI-MS found: 478.1547 [M+1] HPLC: 4.743 1H NMR (700 MHz, DMSO-d6) δ 13.28 (t, J=13.6 Hz, 1H), 11.02 (q, J=7.5, 5.9 Hz, 1H), 9.86 (s, 1H), 8.98 (d, J=52.4 Hz, 1H), 8.57 (d, J=9.4 Hz, 1H), 8.42 (d, J=9.4 Hz, 1H), 8.26 (q, J=9.9, 9.4 Hz, 2H), 7.86 (d, J=11.1 Hz, 1H), 7.64 (s, 2H), 7.58 (d, J=9.2 Hz, 1H), 7.50 (dt, J=27.4, 8.6 Hz, 1H), 6.72 (p, J=8.1 Hz, 1H), 5.23-5.16 (m, 1H), 4.03 (t, J=8.6 Hz, 1H), 2.92 (d, J=16.9 Hz, 14H), 2.26 (d, J=10.2 Hz, 7H), 2.17 (dt, J=18.1, 8.8 Hz, 4H), 1.97 (q, J=8.9 Hz, 2H), 1.48-1.40 (m, 3H). 13C NMR (176 MHz, dmso) δ 169.75, 166.56, 165.36, 159.41, 145.67, 142.08, 141.13, 140.72, 131.91, 128.62, 127.17, 126.78, 126.44, 125.11, 124.85, 124.26, 123.99, 121.10, 118.54, 52.68, 42.73, 20.48, 11.23, 8.81.

### (R,Z)-3-((4-(2-chloroacetamido)-3,5-dimethyl-1H-pyrrol-2-yl)methylene)-N-(1-(3-chlorophenyl)ethyl)-2-oxoindoline-5-carboxamide (9m, RAR-12-89)

To a dried round bottom flask were added 40 mg of 8f, (0.16 mmol) dissolved in 4 mL of THF. The yellow solution was cooled to 0° C. and 0.01 mL (0.18 mmol) of chloroacetylchloride was added dropwise. The solution was allowed to stir at 0° C. for 30 mins. Once complete by TLC, removed solvent under pressure and then added DCM to the dark red residue. The DCM solution was sonicated to give a dark red precipitate. The solid was filtered off and rinsed with THF and DCM to give the desired product as a dark red solid. Result: red solid, 23.4 mg, 28% Molecular Formula: C₂₆H₂₄Cl₂N₄O₃ ESI-MS calc: 510.12 ESI-MS found: 511.1579 [M+1] HPLC: 6.742 1H NMR (500 MHz, DMSO-d6) δ 13.52 (s, 1H), 11.09 (s, 1H), 9.53 (s, 1H), 8.66 (d, J=7.9 Hz, 1H), 8.21 (d, J=1.7 Hz, 1H), 7.68 (dd, J=8.2, 1.7 Hz, 1H), 7.66 (s, 1H), 7.47-7.45 (m, 1H), 7.38-7.36 (m, 2H), 7.29 (dq, J=6.2, 2.2 Hz, 1H), 6.94 (d, J=8.1 Hz, 1H), 5.17 (p, J=7.2 Hz, 1H), 4.26 (s, 2H), 2.20 (d, J=8.6 Hz, 6H), 1.50 (d, J=7.1 Hz, 4H). 13C NMR (126 MHz, dmso) δ 169.51, 165.17, 147.64, 140.23, 132.71, 131.57, 129.96, 127.20, 126.77, 126.30, 125.79, 125.55, 125.23, 124.73, 124.28, 120.82, 117.27, 112.71, 48.03, 42.55, 39.83, 39.67, 39.50, 39.33, 39.17, 39.00, 38.83, 21.96, 11.48, 9.02.

### (R,Z)-3-((4-(2-chloroacetamido)-3,5-dimethyl-1H-pyrrol-2-yl)methylene)-2-oxo-N-(1-(p-tolyl)ethyl)indoline-5-carboxamide (9n, RAR-12-93)

To a dried round bottom flask were added 93 mg of 8g, (0.19 mmol) dissolved in 5 mL of THF. The yellow solution was cooled to 0° C. and 0.1 mL (1.3 mmol) of chloroacetylchloride was added dropwise. The solution was allowed to stir at 0° C. for 30 mins. Once complete by TLC, removed solvent under pressure and then added DCM to the dark red residue. The DCM solution was sonicated to give a dark red precipitate. The solid was filtered off and rinsed with THF and DCM to give the desired product as a dark red solid. Result: red solid, 73.6 mg, 77% Molecular Formula: C₂₇H₂₇ClN₄O₃ ESI-MS calc: 490.18 ESI-MS found 473.05 [M-Cl] HPLC: 6.625 1H NMR (500 MHz, DMSO-d6) δ 13.51 (s, 1H), 11.06 (s, 1H), 9.52 (s, 1H), 8.53 (d, J=8.0 Hz, 1H), 8.20 (s, 1H), 7.71-7.65 (m, 1H), 7.64 (s, 1H), 7.27 (t, J=10.7 Hz, 4H), 7.12 (d, J=7.8 Hz, 6H), 6.92 (d, J=8.1 Hz, 1H), 5.18-5.12 (m, 2H), 2.26 (d, J=3.9 Hz, 8H), 1.48 (d, J=7.1 Hz, 5H).

### (R,Z)-3-((4-(2-chloroacetamido)-3,5-dimethyl-1H-pyrrol-2-yl)methylene)-N-(1-(4-chlorophenyl)ethyl)-2-oxoindoline-5-carboxamide (RAR-13-19, 273583, 90)

Synthesized using the protocol described in 8h. Yields a brown solid, 43.5 mg, 100%. Molecular Formula: C₂₆H₂₄Cl₂N₄O₃ ESI-MS calc: 510.12 ESI-MS found: 511.12917 [M+1] HPLC: 6.723. 1H NMR (499 MHz, DMSO-d6) δ 13.52 (s, 1H), 11.08 (d, J=7.8 Hz, 1H), 9.53 (s, 1H), 8.64 (dd, J=7.9, 5.3 Hz, 2H), 8.20 (d, J=1.5 Hz, 1H), 7.67 (dt, J=8.0, 2.1 Hz, 2H), 7.65 (s, 1H), 7.44-7.40 (m, 4H), 7.40-7.37 (m, 5H), 6.93 (d, J=8.1 Hz, 1H), 5.16 (dq, J=13.7, 7.0 Hz, 1H), 4.26 (d, J=2.8 Hz, 2H), 2.20 (d, J=8.4 Hz, 6H), 1.49 (d, J=7.1 Hz, 3H). 13C NMR (126 MHz, dmso) δ 170.31, 169.68, 165.92, 165.35, 144.15, 140.36, 131.73, 130.99, 128.11, 128.01, 127.44, 126.92, 126.09, 125.76, 125.35, 124.44, 123.36, 122.09, 121.43, 120.99, 120.43, 117.37, 108.64, 55.81, 42.72, 22.10, 11.65, 9.21.

### (R,Z)-3-((4-(2-bromoacetamido)-3,5-dimethyl-1H-pyrrol-2-yl)methylene)-N-(1-(4-fluorophenyl)ethyl)-2-oxoindoline-5-carboxamide (9p, RAR-13-26, CCG-359090)

This material was prepared using the protocol described for 8c. Yields a bright orange solid, 13.7 mg, 23%. HRMS: 541.1106 [M+1, Br⁸¹], HPLC: 6.449. 1H NMR (700 MHz, DMSO-d6) δ 13.52 (s, 1H), 11.08 (s, 1H), 9.60 (s, 1H), 8.61 (d, J=8.1 Hz, 1H), 8.20 (s, 1H), 7.68 (d, J=8.4 Hz, 1H), 7.65 (s, 1H), 7.44 (t, J=4.8 Hz, 2H), 7.17-7.13 (m, 2H), 6.93 (dd, J=8.2, 2.5 Hz, 1H), 5.19 (q, J=7.4 Hz, 1H), 4.26 (d, J=2.4 Hz, 1H), 4.03 (d, J=2.4 Hz, 1H), 2.22-2.18 (m, 6H), 1.51-1.48 (m, 3H). 13C NMR (176 MHz, dmso) δ 170.07, 167.64, 166.24, 162.00, 155.00, 144.70, 140.72, 132.05, 129.71, 128.41, 127.92, 124.56, 122.76, 121.42, 117.77, 115.96, 115.26, 109.01, 48.19, 29.73, 22.66, 12.01, 9.54.

### (Z)—N-benzyl-3-((4-(2-chloroacetamido)-3,5-dimethyl-1H-pyrrol-2-yl)methylene)-2-oxoindoline-5-carboxamide (RAR-13-3, 273561, 9q)

Synthesized using protocol described in 9i. Yields an orange solid, 18.2 mg, 21%. Molecular Formula: C₂₅H₂₃ClN₄O₃ ESI-MS calc: 462.15 ESI-MS found: 463.1529 [M+1] HPLC: 6.177. 1H NMR (700 MHz, DMSO-d6) δ 13.49 (s, 1H), 11.08 (s, 1H), 9.52 (s, 1H), 8.85 (dt, J=12.4, 6.1 Hz, 1H), 8.26 (d, J=1.7 Hz, 1H), 7.78 (d, J=9.4 Hz, 1H), 7.71 (dd, J=8.2, 1.7 Hz, 1H), 7.64 (s, 1H), 7.35-7.32 (m, 4H), 7.24 (tt, J=6.2, 3.0 Hz, 2H), 6.94 (d, J=8.1 Hz, 1H), 4.51 (d, J=6.0 Hz, 2H), 4.26 (s, 2H), 2.21 (s, 3H), 2.18 (s, 3H). 13C NMR (176 MHz, dmso) δ 170.18, 166.91, 165.85, 140.89, 140.42, 132.22, 128.74, 128.72, 128.70, 127.98, 127.78, 127.68, 127.67, 127.62, 127.38, 127.15, 126.21, 125.93, 124.95, 124.54, 123.95, 121.48, 117.63, 113.41, 109.28, 68.98, 56.32, 43.22, 43.09, 43.03, 40.37, 40.25, 40.13, 40.01, 39.89, 39.77, 39.65, 36.08, 32.59, 30.08, 12.14, 11.67, 9.92, 9.65.

### (Z)-3-((4-(2-chloroacetamido)-3,5-dimethyl-1H-pyrrol-2-yl)methylene)-2-oxo-N-(2-phenylpropan-2-yl)indoline-5-carboxamide (RAR-13-15, 273564, 9r)

Synthesized using the protocol described in 9i. Yields a brown solid, 14.2 mg, 15%. Molecular Formula: C₂₇H₂₇ClN₄O₃ ESI-MS calc: 490.18 ESI-MS found: 491.1832 [M+1], 513.1656 [M+Na] HPLC: 6.63. 1H NMR (500 MHz, DMSO-d6) δ 13.52 (s, 1H), 11.06 (s, 1H), 9.53 (s, 1H), 8.24 (s, 1H), 8.19 (d, J=1.8 Hz, 1H), 7.68 (s, 1H), 7.64 (dd, J=8.1, 1.7 Hz, 1H), 7.40 (dd, J=7.9, 1.8 Hz, 3H), 7.28 (t, J=7.8 Hz, 2H), 7.16 (dd, J=8.3, 6.3 Hz, 1H), 6.91 (d, J=8.1 Hz, 1H), 4.26 (s, 2H), 2.20 (d, J=8.2 Hz, 6H), 1.69 (s, 6H). 13C NMR (126 MHz, dmso) δ 170.12, 166.37, 165.75, 148.67, 143.56, 140.59, 132.03, 128.86, 128.24, 127.26, 126.00, 125.65, 125.11, 124.86, 124.66, 121.37, 117.90, 108.94, 55.69, 43.14, 30.13, 12.07, 9.61.

### (R,Z)-3-((4-(2-chloroacetamido)-3,5-dimethyl-1H-pyrrol-2-yl)methylene)-N-(2-methyl-1-phenylpropyl)-2-oxoindoline-5-carboxamide (RAR-13-7, 273562, 9s)

Synthesized using the protocol described in 9i. Yields an orange solid, 5.5 mg, 5.8%. Molecular Formula: C₂₈H₂₉ClN₄O₃ ESI-MS calc: 504.19 ESI-MS found: 505.1997 HPLC: 6.803 1H NMR (700 MHz, DMSO-d6) δ 13.52 (s, 1H), 11.06 (s, 1H), 9.52 (s, 1H), 8.53 (d, J=8.9 Hz, 1H), 8.14 (d, J=1.6 Hz, 1H), 7.67-7.62 (m, 2H), 7.43-7.40 (m, 2H), 7.32 (t, J=7.6 Hz, 2H), 7.22 (t, J=7.3 Hz, 1H), 6.92 (d, J=8.1 Hz, 1H), 4.69 (t, J=9.2 Hz, 1H), 4.26 (s, 2H), 2.20 (d, J=10.3 Hz, 6H), 1.04 (d, J=6.6 Hz, 4H), 0.73 (d, J=6.7 Hz, 4H). 13C NMR (126 MHz, dmso) δ 170.14, 166.69, 165.80, 151.43, 143.78, 140.68, 132.14, 128.47, 128.45, 127.89, 127.78, 127.38, 127.05, 125.84, 124.90, 124.74, 121.43, 118.01, 108.97, 60.35, 43.19, 32.97, 20.45, 12.12, 9.62.

### (Z)-3-((4-(2-chloroacetamido)-3,5-dimethyl-1H-pyrrol-2-yl)methylene)-2-oxo-N-(3-phenyloxetan-3-yl)indoline-5-carboxamide (RAR-13-11, 273563, 9t)

Synthesized using the protocol described in 9i. Yields a red solid, 5.0 mg, 21%. Molecular Formula: C₂₇H₂₅ClN₄O₄ ESI-MS calc: 504.16 ESI-MS found: 505.1619 HPLC: 5.332 1H NMR (700 MHz, DMSO-d6) δ 13.52 (s, 1H), 11.28 (s, 1H), 9.54 (d, J=13.2 Hz, 1H), 8.43 (s, 1H), 7.84 (s, 2H), 7.77 (d, J=13.4 Hz, 2H), 7.52 (t, J=9.1 Hz, 4H), 7.39 (dt, J=14.7, 7.8 Hz, 5H), 7.31 (dd, J=16.7, 7.7 Hz, 3H), 7.19 (s, 1H), 7.11 (s, 1H), 7.04 (s, 2H), 4.85 (d, J=9.0 Hz, 2H), 4.45 (t, J=9.9 Hz, 2H), 4.27 (d, J=3.1 Hz, 2H), 2.22 (d, J=4.8 Hz, 6H). 13C NMR (126 MHz, dmso) δ 170.16, 167.57, 165.90, 154.45, 143.80, 130.21, 129.17, 129.09, 129.04, 128.64, 126.55, 126.07, 125.78, 125.47, 125.31, 122.08, 121.39, 120.35, 115.60, 110.34, 78.59, 72.40, 43.21, 11.78, 9.23.

### (Z)-3-((4-((E)-2-cyano-4,4-dimethylpent-2-enamido)-3,5-dimethyl-1H-pyrrol-2-yl)methylene)-2-oxo-N—((R)-1-phenylethyl)indoline-5-carboxamide (10a, R,AR-12-61)

To a sealed tube were added 30 mg of 9g (0.064 mmol), 0.05 mL of pivaldehyde (0.45 mmol), and 0.10 mL of piperidine (1.0 mmol). All materials were then dissolved in 3 mL of abs. EtOH. The solution was then heated to 75° C. for 12 h. Once complete, the reaction was cooled to rt and then solvent was removed under pressure, evaporating the crude material onto silica gel. The material was purified by column chromatography (50% Acetone/Hexanes) to give the desired product. Yields a yellow solid, 14 mg, 41%. MS: 536.1375 [M+1], 558 [M+Na] HPLC: 7.69 1H NMR (700 MHz, DMSO-d6) δ 13.54 (s, 1H), 11.09 (s, 1H), 9.57 (d, J=11.5 Hz, 1H), 8.60 (s, 2H), 8.22 (s, 1H), 7.95 (s, 1H), 7.69 (d, J=8.4 Hz, 1H), 7.66 (d, J=9.8 Hz, 1H), 7.41 (d, J=7.7 Hz, 3H), 7.33 (t, J=7.5 Hz, 3H), 7.23 (d, J=7.9 Hz, 2H), 6.93 (d, J=8.4 Hz, 1H), 5.19 (s, 1H), 2.22-2.17 (m, 6H), 1.50 (d, J=7.2 Hz, 4H), 1.15 (d, J=9.5 Hz, 9H), 1.04 (d, J=6.1 Hz, 3H). 13C NMR (176 MHz, dmso) δ 170.08, 167.90, 166.20, 160.95, 153.74, 145.51, 141.02, 140.65, 135.87, 132.53, 128.55, 128.02, 126.86, 126.46, 124.51, 123.07, 120.43, 117.75, 115.67, 98.56, 56.19, 28.98, 25.24, 22.64, 12.00, 9.70.

### (Z)-3-((3,5-dimethyl-4-(oxirane-2-carboxamido)-1H-pyrrol-2-yl)methylene)-2-oxo-N—((R)-1-phenylethyl)indoline-5-carboxamide (10b R,AR-12-96)

To a flask were added 36.4 mg of RAR-12-95, and 47.2 mg of K2CO3. All materials were brought up in 3.0 mL of Acetone and then refluxed (60 C) for 30 mins. Cooled to rt and then checked by TLC, not complete, so run for another 60 mins. HPLC confirms still not done. MS looks like mostly the starting material. Heated up to 70 C for another 1 h, TLC shows its not complete. So added cat. KI and heated back up to 70 C for 45 mins. Cooled to rt and check again by TLC/HPLC. Not complete (even after o/n run→76% complete) Added 2 more equiv of KI and 1 mL of MeCN (to solubilize) and then heated back to 70 C, after 1 h, if not complete, work up and try to isolate. Quenched with water (1×30 mL) and then extracted with EtOAc (2×25 mL). Dried over Na2SO4 and then purified by column chromatography 25-100% Acetone/Hexanes Collected F19-23 as the potential product and F24-31 as the starting material. Double check by MS before NMR Result: yellow residue, 3.0 mg, 6% Molecular Formula: C₂₇H₂₆ClN₄O₄ ESI-MS calc: 470.20 ESI-MS found: 471.2158 [M+1] HPLC: 6.591 1H NMR (700 MHz, Acetone-d6) δ 13.62 (s, 1H), 10.01 (s, 1H), 8.24-8.22 (m, 2H), 7.83 (d, J=8.3 Hz, 1H), 7.75 (dt, J=8.1, 2.2 Hz, 1H), 7.69 (s, 1H), 7.46 (d, J=8.0 Hz, 3H), 7.33 (t, J=7.7 Hz, 3H), 7.23 (t, J=7.3 Hz, 1H), 6.99 (dd, J=8.3, 5.5 Hz, 1H), 5.33 (q, J=7.3 Hz, 2H), 3.91 (s, 2H), 3.79 (s, 2H), 2.30 (s, 2H), 2.25 (d, J=2.7 Hz, 3H), 2.19 (d, J=5.5 Hz, 2H), 2.14 (d, J=5.1 Hz, 3H), 1.56 (dd, J=7.1, 1.3 Hz, 3H). 13C NMR (126 MHz, dmso) δ 171.84, 169.70, 167.77, 152.56, 145.07, 140.26, 132.32, 128.18, 127.58, 127.45, 126.50, 126.09, 124.43, 121.76, 120.08, 118.18, 108.61, 54.27, 54.08, 48.37, 22.28, 11.87, 9.44.

### INCORPORATION BY REFERENCE

The entire disclosure of each of the patent documents and scientific articles referred to herein is incorporated by reference for all purposes. The following references are herein incorporated by reference in their entireties:

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound encompassed within Formula I: 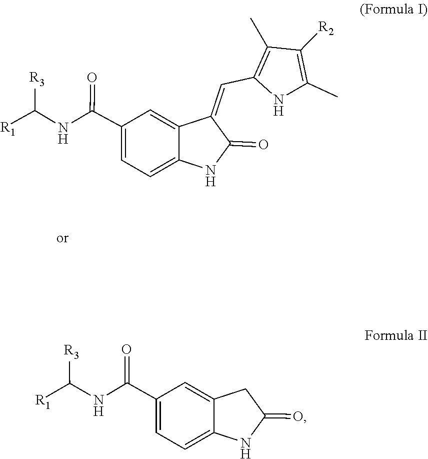
including pharmaceutically acceptable salts, solvates, and/or prodrugs thereof, wherein R1, R2 and R3 independently include any chemical moiety that permits the resulting compound to inhibit GRK5 activity and/or inhibit GRK5 subfamily (e.g., GRK4, GRK5, GRK6) member activity.

2. The compound of claim 1, wherein each of R1, R2 and R3 independently include any chemical moiety that permits the resulting compound to have one or more of the following capabilities:
inhibit GRK5 activity while not affecting GRK2 activity;
inhibit histone deacetylase 5 (HDAC5) phosphorylation;
inhibit GRK5 activity through binding a GRK5 protein at the CYS474 position;
bind a GRK5 protein as the CYS474 position;
inhibit GRK5 related interaction with IκBα which thereby inhibits NFKB-mediated transcriptional responses; and
inhibit GRK5 related phosphorylation of p53 and regulates p53-mediated apoptosis in response to DNA damage.

3. The compound of claim 1, wherein R1 is selected from hydrogen,

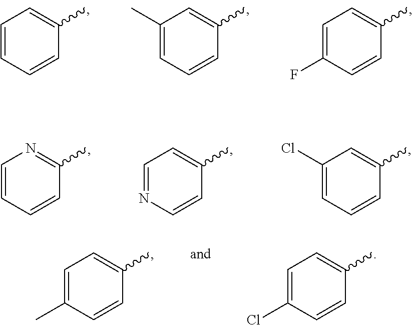

4. The compound of claim 1, wherein R2 is selected from hydrogen,

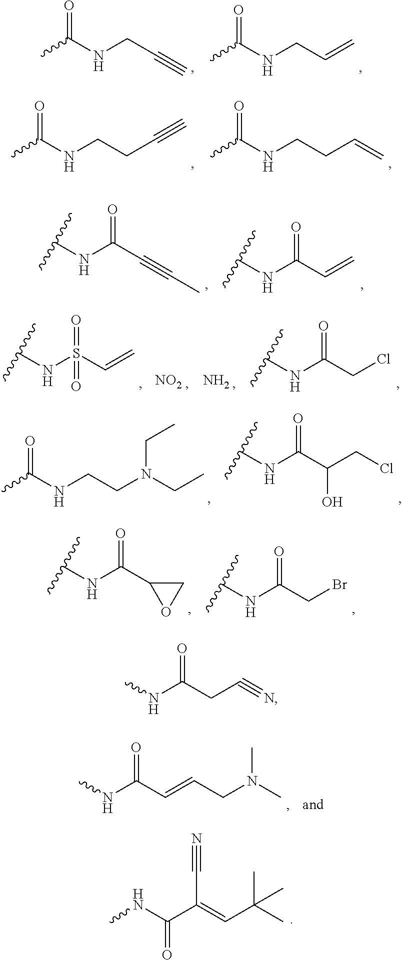

5. The compound of claim 1, wherein R3 is selected from hydrogen, 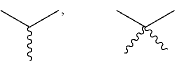
(e.g., such that the resulting compound is either H or 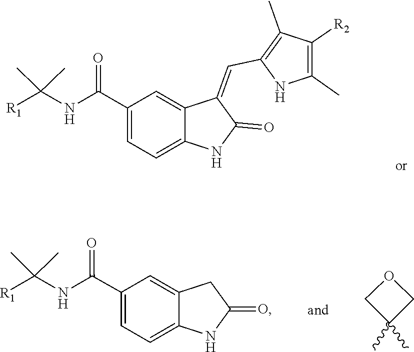
(e.g., such that the resulting compound is either

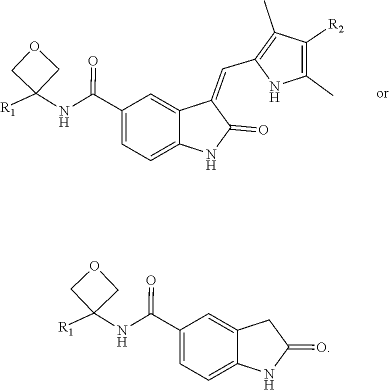

6. The compound of claim 1, wherein said compound is selected from the group of compounds recited in Tables 1 and 2 and Example 1.

7. A pharmaceutical composition comprising a compound of claim 1.

8. A method of treating, ameliorating, or preventing a disorder related to GRK5 activity in a patient comprising administering to said patient a therapeutically effective amount of the pharmaceutical composition of claim 7.

9. The method of claim 8, wherein said disorder related to GRK5 activity is a heart condition or cancer.

10. The method of claim 9, wherein said patient is a human patient.

11. The method of claim 8, further comprising administering to said patient one or more agents for treating a heart condition and/or cancer.

12. The method of claim 8, wherein the heart condition is one or more of cardiac failure, cardiac hypertrophy, and hypertension.

13. The method of claim 8, wherein the cancer is breast cancer.

14. A kit comprising a compound of claim 1 and instructions for administering said compound to a patient having a disorder related to GRK5 activity.

15. A method for inhibiting GRK5 related activity in a subject, comprising administering to the subject a compound of claim 1.

16. The method of claim 15, wherein the subject is a human patient.

17. The method of claim 15, wherein the subject is suffering from a heart condition and/or cancer.

18. The method of claim 17, wherein the heart condition is one or more of cardiac failure, cardiac hypertrophy, and hypertension.

19. The method of claim 17, wherein the cancer is breast cancer.


SEQUENCE LISTING (1 sequence)

SEQ ID NO: 1 - protein, 13 aa, Artificial sequence
  synthetic
KRREILSRRPSYR